(19) United States
(12) Patent Application Publication
(10) Pub. No.: US 2019/0135787 A1
(43) Pub. Date: May 9, 2019

(54) PYRIMIDINE COMPOSITIONS, ULTRA-PURE COMPOSITIONS AND SALTS THEREOF, METHODS OF MAKING THE SAME, AND METHODS OF USING THE SAME FOR TREATING HISTAMINE H4 RECEPTOR (H4) MEDIATED DISEASES AND CONDITIONS

(71) Applicant: Novartis AG, Basel (CH)

(72) Inventors: Zhijian ZHU, Wayland, MA (US); Helen BARKER, Oxfordshire (GB); Michael YEADON, Kent (GB); Wai LIU, Kent (GB)

(21) Appl. No.: 15/770,825

(22) PCT Filed: Oct. 25, 2016
(86) PCT No.: PCT/EP2016/075708

§ 371 Date: Apr. 25, 2018

Related U.S. Application Data
(60) Provisional application No. 62/246,482, filed on Oct. 26, 2015.
(60) Provisional application No. 62/329,091, filed on Apr. 28, 2016.
(60) Provisional application No. 62/359,066, filed on Jul. 6, 2016.

(51) Int. Cl. C07D 403/04; A61K 31/506; A61K 9/20; A61P 17/04

(52) U.S. Cl. CPC C07D 403/04; A61K 31/506; A61K 9/2018; C07B 2200/13; A61K 9/2059; A61K 9/2054; A61P 17/04; A61K 9/2009

(57) ABSTRACT

The present application relates to ultra-pure compositions containing N⁴-(cyclopropylmethyl)-6-[(3R)-3-(methylamino)pyrrolidin-1-yl]pyrimidine-2,4,-diamine tartrate dihydrate, methods of making the same, formulations containing the same, methods of using the same to treat H₄-mediated diseases and conditions, and alternative salt forms thereof.

### RELATED APPLICATIONS

This application is a National Stage of International Application No. PCT/EP2016/075708, filed on Oct. 25, 2016, which claims the benefit of and priority to U.S. Provisional Application No. 62/246,482, filed Oct. 26, 2015, U.S. Provisional Application No. 62/329,091, filed Apr. 28, 2016, and U.S. Provisional Application No. 62/359,066, filed Jul. 6, 2016. Each of these documents is incorporated by reference herein in its entirety for all purposes.

### FIELD OF THE INVENTION

The present application relates to ultra-pure compositions containing N⁴-(cyclopropylmethyl)-6-[(3R)-3-(methylamino)pyrrolidin-1-yl]pyrimidine-2,4-diamine tartrate dihydrate as well as methods of making the same, methods of using the same to treat H₄-mediated diseases and conditions, and alternative salt forms thereof.

### BACKGROUND OF THE INVENTION

Histamine, a heterocyclic amine that is released by a variety of inflammatory cell types when tissue is injured or in allergic and inflammatory reactions, can play a role in a variety of conditions and exerts its biological effects by binding to and activating four distinct separate rhodopsin-like G protein-coupled receptors (histamine H₁ receptor, histamine H₂ receptor, histamine H₃ receptor, and histamine H₄ receptor) that each produce a functional response via different mechanisms.

The histamine H₄ receptor is a 390 amino-acid, seven-transmembrane G protein coupled receptor with approximately 40% homology to the histamine H₃ receptor. Histamine H₄ receptors (HH4R or H₄) couple to G proteins to inhibit adenylyl cyclase.

While the histamine H₄ receptor is highly expressed in the bone marrow and white blood cells, it is also expressed in the colon, liver, lung, small intestine, spleen, testes, thymus, tonsils, and trachea. Thus, the H₄ receptor is a potential target in allergic and inflammatory diseases. Moreover, activation of the H₄ receptor can also enhance the activity of other chemoattractants, such as chemokines on eosinophils and upregulate adhesion of molecules.

In contrast to the H₃ receptor, which is primarily located in the brain, the H₄ receptor is expressed at greater levels in eosinophils and mast cells, among other inflammatory cells. Thus, H₄ receptor ligands should be suitable for the treatment of various inflammatory disorders, including, but not limited to, inflammatory bowel disease, Crohn's disease, colitis ulcerosa, dermatitis, psoriasis, conjunctivitis, rheumatoid arthritis, respiratory diseases such as adult respiratory distress syndrome, acute respiratory distress syndrome, bronchitis, chronic bronchitis, chronic obstructive pulmonary disease, cystic fibrosis, asthma, emphysema, rhinitis, chronic sinusitis, allergy, allergy-induced airway responses, allergic rhinitis, viral rhinitis, non-allergic rhinitis, perennial and seasonal rhinitis, nasal congestion and allergic congestion.

An overview of the current advances in H₄ ligand research and patenting is given in Carlberg, C. et al. Expert Opin. Ther. Patents (2003) 13(6), which is incorporated herein by reference. Examples of Histamine H₄ receptor ligands can be found in WO 02/072548, WO 04/022537, Terzioglu et al., J. Bioorg. Med. Chem. Left. 14 (2004), 5251-5256, and U.S. Pat. No. 7,943,628, each of which are herein incorporated by reference.

Although H₄ ligands are known, there is still a need to further provide new H₄ ligands that are good drug candidates. In particular, preferred compounds should bind potently to the histamine H₄ receptor, while showing little affinity for other receptors. They should also be well absorbed from the gastrointestinal tract, be metabolically stable, possess favorable pharmacokinetic properties, be non-toxic, and demonstrate few side-effects.

### SUMMARY OF THE INVENTION

Provided herein are compositions containing or related to N⁴-(cyclopropylmethyl)-6-[(3R)-3-(methylamino)pyrrolidin-1-yl]pyrimidine-2,4-diamine,

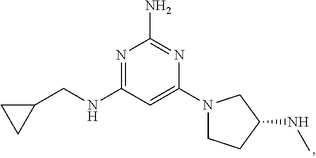 [SMILES: CN[C@@H]1CCN(c2cc(NCC3CC3)nc(N)n2)C1]

and salts, solvates, or hydrates thereof. This compound is also known in the art as PF-03893787, PF-3893787, ZPL-389 and ZPL-3893787, and these terms are used interchangeably herein.

Provided herein are compositions containing N⁴-(cyclopropylmethyl)-6-[(3R)-3-(methylamino)pyrrolidin-1-yl]pyrimidine-2,4-diamine tartrate dihydrate, wherein the composition is at least 98% pure (e.g., at least 98, 98.1, 98.2, 98.3, 98.4, 98.5, 98.6, 98.7, 98.8, 98.9, 99.0, 99.1, 99.2, 99.3, 99.4, 99.5, 99.6, 99.7, 99.8, 99.9, or more % pure). This compound is also known in the art as PF-03893787-18, PF-3893787-18 and ZPL-3893787-18.

Also provided are compositions containing N⁴-(cyclopropylmethyl)-6-[(3R)-3-(methylamino)pyrrolidin-1-yl]pyrimidine-2,4-diamine tartrate dihydrate, wherein the composition further comprises less than 1% (i.e., less than 0.95%, 0.90%, 0.85%, 0.80%, 0.75%, 0.70%, 0.65%, 0.60%, 0.55%, 0.50%, 0.45%, 0.40%, 0.35%, 0.30%, 0.29%, 0.28%, 0.27%, 0.26%, 0.25%, 0.24%, 0.23%, 0.22%, 0.21%, 0.20%, 0.15%, 0.10%, or 0.05%) of 4-N-butyl-6-[(3-(methylamino)pyrrolidin-1-yl]pyrimidine-2,4-diamine i.e.,

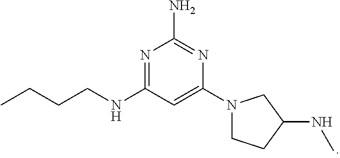 [SMILES: CCCCNc1cc(N2CCC(NC)C2)nc(N)n1]

For example, in some embodiments, this impurity is 4-N-butyl-6-[(3R)-3-methylamino)pyrrolidin-1-yl]pyrimidine-2,4-diamine.

In one embodiment, the composition contains less than 0.26% of the impurity.

These compositions may additionally contain less than 0.5% (i.e., less than 0.45%, 0.4%, 0.35%, 0.3%, 0.25%, 0.2%, 0.15%, 0.1%, or 0.05%) methanol. By way of non-limiting example, the compositions may contain between about 0.1% to about 0.5% methanol, for example, between 0.1-0.2%, 0.1-0.3%, 0.1-0.4%, 0.2-0.3%, 0.2-0.4%, 0.2-0.5%, 0.3-0.4%, 0.3-0.5%, or 0.4-0.5% methanol.

Any of the compositions described herein may contain a polymorph of N⁴-(cyclopropylmethyl)-6-[(3R)-3-(methylamino)pyrrolidin-1-yl]pyrimidine-2,4-diamine tartrate dihydrate distinguished by PXRD peaks at about 6.7, 9.2, 22.4, and 24.4 degrees 2-theta.

In some embodiments, the polymorph is distinguished by two additional peaks at about 13.5 and 18.7 degrees 2-theta. In further embodiments, the polymorph is distinguished by four additional peaks at about 20.9, 21.4, 26.8, and 30.0 degrees 2-theta. In still further embodiments, the polymorph is distinguished by four additional peaks at about 11.4, 15.6, 25.0, and 26.1 degrees 2-theta. Finally, in still further embodiments, the polymorph is distinguished by three additional peaks at about 17.0, 21.8, and 22.0 degrees 2-theta.

Also provided are compositions containing N⁴-(cyclopropylmethyl)-6-[(3R)-3-(methylamino)pyrrolidin-1-yl]pyrimidine-2,4-diamine tartrate dihydrate, wherein the composition is at least 98% pure and/or that contain less than 1% of 4-N-butyl-6-[(3-(methylamino)pyrrolidin-1-yl]pyrimidine-2,4-diamine that contain a polymorph of N⁴-(cyclopropylmethyl)-6-[(3R)-3-(methylamino)pyrrolidin-1-yl]pyrimidine-2,4-diamine tartrate dihydrate distinguished by PXRD peaks at about 17.0, 21.8, and 26.1 degrees 2-theta.

Any of the compositions described herein can be combined with one or more pharmaceutically acceptable carrier(s) and/or diluent(s) to form a pharmaceutical composition.

Likewise, dosage forms containing an effective amount of any of the compositions or pharmaceutical compositions described herein are also provided. By way of non-limiting example, the dosage form may be powder-in-capsule forms, capsules, tablets, liquids, powders, lozenges, chews, multi- and nano-particulates, gels, solid solutions, liposomes, nanoparticles, films, ovules, sprays, injectables, and liquid formulations. In one embodiment, the dosage form is a powder-in-capsule form. In another embodiment, the dosage form is a tablet.

Also provided are compositions, pharmaceutical compositions, or dosage forms for treatment of an H₄ mediated disease or condition as well as methods of treating an H₄ mediated disease or condition by administering an effective amount of any of the compositions, pharmaceutical compositions, and/or dosage forms described herein to a patient in need thereof.

Further provided are compositions, pharmaceutical compositions, or dosage forms of the invention for use in treating an H₄ mediated disease or condition.

By way of non-limiting example, the H₄ mediated disease or condition is selected from the group consisting of inflammatory skin diseases (i.e., atopic dermatitis or psoriasis), pruritic diseases (i.e., urticaria or uraemic pruritus), respiratory diseases (i.e., asthma, chronic obstructive airway disease, or allergic rhinitis), cardiac diseases (i.e., myocardial ischaemia), inflammatory diseases of the gastrointestinal tract (i.e., Crohn's disease or colitis ulcerosa), cancer, joint diseases (i.e., rheumatoid arthritis or psoriatic arthritis), kidney diseases (i.e., diabetic nephropathy), pain disorders (i.e., inflammatory pain or neuropathic pain), overactive bladder conditions, vestibular disorders (i.e., vertigo or tinnitus), macular degenerative disorders, inflammatory eye diseases (i.e., conjunctivitis or uveitis), and other diseases involving immune and inflammatory disorders (i.e., multiple sclerosis, mastocytosis, or inflammatory or systemic lupus erythematosus).

In some embodiments, the H₄ mediated disease or condition is selected from the group consisting of atopic dermatitis, bullous disorders, collagenoses, psoriasis, psoriatic lesions, seborrheic dermatitis or contact dermatitis, eczema, urticaria, pruritus, uraemic pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, squamous cell carcinoma or melanoma.

In some preferred embodiments, the disease or condition is psoriasis, atopic dermatitis, or other pruritic conditions.

The compositions, pharmaceutical compositions, or dosage forms can be administered to the patient via an oral, topical, intravenous, intraarterial, intraocular, intraperitoneal, intrathecal, intraventricular, intraurethral, intrasternal, intracranial, intramuscular, or subcutaneous route of administration.

For example, the compositions, pharmaceutical compositions, or dosage forms can be administered to the patient once daily.

The compositions, pharmaceutical compositions, or dosage forms can be administered at a dose of from about 1 mg to about 60 mg (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg).

In various embodiments, the compositions, pharmaceutical compositions, or dosage forms can be administered at a dose of from about 10 to about 60 mg; at a dose of from about 5 mg to about 50 mg; at a dose of from about 1 mg to about 10 mg; at a dose of from about 3 mg to about 15 mg; at a dose of from about 5 mg to about 20 mg; and/or at a dose of from about 10 mg to about 30 mg.

Any of the compositions, pharmaceutical compositions, or dosage forms can be administered intravenously, subcutaneously, or intraocularly, at a dosage of from about 0.005 to about 100 mg/ml (e.g., about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml).

In various embodiments, the compositions, pharmaceutical compositions, or dosage forms can be administered at a dose of from about 0.05 to about 100 mg/ml; at a dose of from about 0.01 to about 90 mg/ml; at a dose of from about 0.005 to about 10 mg/ml; at a dose of from about 0.05 to about 15 mg/ml; at a dose of from about 0.5 to about 20 mg/ml; at a dose of from about 10 to about 30 mg/ml.

Any of the compositions, pharmaceutical compositions, or dosage forms can be administered to the patient with one or more additional therapeutic agents. By way of non-limiting example, the one or more additional therapeutic agents are selected from Histamine H₁ receptor antagonists (i.e., fexofenadine, cetirizine, levocetrizine, loratadine, desloratadine, mepyramine, and diphenhydramine); Histamine H₃ receptor antagonists; Histamine H₂ receptor antagonists; leukotriene antagonists (i.e., montelukast, zafirlukast, and pranlukast); phosphodiesterase inhibitors (i.e., PDE4 phosphodiesterase inhibitors such as apremilast or roflumilast); neurotransmitter re-uptake inhibitors; 5-lipooxygenase (5-LO) inhibitors; 5-lipoxygenase activating protein (FLAP) inhibitors; α₁- and α₂-adrenoceptor agonist vasoconstrictor sympathomimetic agents; muscarinic M₃ receptor antagonists or anticholinergic agents; β₂-adrenoceptor agonists; dual acting β₂/M₃ agents; xanthines; non-steroidal anti-inflammatories; ketotifen; COX-1 inhibitors (NSAIDs) and COX-2 selective inhibitors; oral, inhaled intranasal and topical glucocorticosteroids; monoclonal antibodies active against endogenous inflammatory entities; anti-tumor necrosis factor (anti-TNF-α) agents; adhesion molecule inhibitors including VLA-4 antagonists; kinin-B₁- and B₂-receptor antagonists; immunosuppressive agents; inhibitors of matrix metalloproteases (MMPs); tachykinin NK₁, NK₂ and NK₃ receptor antagonists; elastase inhibitors; adenosine A2a receptor agonists; inhibitors of urokinase; compounds that act on dopamine receptors; modulators of the NFκb pathway; agents that can be classed as mucolytics or anti-tussive agents; antibiotics; modulators of cytokine signaling pathways; modulators of the prostaglandin pathways; antagonists of chemokine receptors CXCR1 and CXCR2; antagonists of chemokine receptors CCR3, CCR4 and CCR5; inhibitors of cytosolic and soluble phospholipase A₂ (cPLA₂ and sPLA₂); inhibitors of phosphoinositide-3-kinase; HDAC inhibitors; p38 inhibitors; CXCR2 antagonists; calcineurin inhibitors; anti-interleukin 17 (anti-IL-17) agents; anti-interleukin 4 receptor (anti-IL4R) agents; anti-interleukin 31 (anti-IL-31) agents; CRTH2 antagonists (i.e., ADC3680, NVP-QAV680, and OC459); and combinations thereof.

Also provided are compositions containing N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine for treatment of an H₄ mediated condition in combination with one or more additional therapeutic agents selected from the group consisting of calcineurin inhibitors, anti-interleukin 17 (anti-IL-17) agents, anti-interleukin 4 receptor (anti-IL-4R) agents, anti-interleukin-31 (anti-IL-31) agents, and combinations thereof to a patient in need thereof.

Likewise, also provided are methods of treating an H₄ mediated condition by administering an effective amount of a composition containing N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine in combination with one or more additional therapeutic agents selected from the group consisting of calcineurin inhibitors, anti-interleukin 17 (anti-IL-17) agents, anti-interleukin 4 receptor (anti-IL-4R) agents, anti-interleukin-31 (anti-IL-31) agents, and combinations thereof to a patient in need thereof.

Also provided is the composition comprising N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine for use in a method of treating an H₄ mediated condition, wherein the composition is administered simultaneously, separately or sequentially in combination with one or more additional therapeutic agents selected from the group consisting of calcineurin inhibitors, anti-interleukin 17 (anti-IL-17) agents, anti-interleukin 4 receptor (anti-IL-4R) agents, anti-interleukin-31 (anti-IL-31) agents, and combinations thereof to a patient in need thereof.

By way of non-limiting example, the H₄ mediated disease or condition is selected from the group consisting of inflammatory skin diseases (i.e., atopic dermatitis or psoriasis), pruritic diseases (i.e., urticaria or uraemic pruritus), respiratory diseases (i.e., asthma, chronic obstructive airway disease, or allergic rhinitis), cardiac diseases (i.e., myocardial ischaemia), inflammatory diseases of the gastrointestinal tract (i.e., Crohn's disease or colitis ulcerosa), cancer, joint diseases (i.e., rheumatoid arthritis or psoriatic arthritis), kidney diseases (i.e., diabetic nephropathy), pain disorders (i.e., inflammatory pain or neuropathic pain), overactive bladder conditions, vestibular disorders (i.e., vertigo or tinnitus), macular degenerative disorders, inflammatory eye diseases (i.e., conjunctivitis or uveitis), and other diseases involving immune and inflammatory disorders (i.e., multiple sclerosis, mastocytosis, or inflammatory or systemic lupus erythematosus).

In some embodiments, the H₄ mediated disease or condition is selected from the group consisting of atopic dermatitis, bullous disorders, collagenoses, psoriasis, psoriatic lesions, seborrheic dermatitis or contact dermatitis, eczema, urticaria, uraemic pruritus, pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, squamous cell carcinoma or melanoma.

In some preferred embodiments, the disease or condition is psoriasis, atopic dermatitis, or other pruritic conditions.

The compositions, pharmaceutical compositions, or dosage forms can be administered to the patient via an oral, topical, intravenous, intraarterial, intraocular, intraperitoneal, intrathecal, intraventricular, intraurethral, intrasternal, intracranial, intramuscular, or subcutaneous route of administration.

For example, the compositions, pharmaceutical compositions, or dosage forms can be administered to the patient once daily.

The compositions, pharmaceutical compositions, or dosage forms can be administered at a dose of from about 1 mg to about 60 mg (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg).

In various embodiments, the compositions, pharmaceutical compositions, or dosage forms can be administered at a dose of from about 10 to about 60 mg; at a dose of from about 5 mg to about 50 mg; at a dose of from about 1 mg to about 10 mg; at a dose of from about 3 mg to about 15 mg; at a dose of from about 5 mg to about 20 mg; and/or at a dose of from about 10 mg to about 30 mg.

Any of the compositions, pharmaceutical compositions, or dosage forms can be administered intravenously, subcutaneously, or intraocularly, at a dosage of from about 0.005 to about 100 mg/ml (e.g., about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml).

In various embodiments, the compositions, pharmaceutical compositions, or dosage forms can be administered at a dose of from about 0.05 to about 100 mg/ml; at a dose of from about 0.01 to about 90 mg/ml; at a dose of from about 0.005 to about 10 mg/ml; at a dose of from about 0.05 to about 15 mg/ml; at a dose of from about 0.5 to about 20 mg/ml; at a dose of from about 10 to about 30 mg/ml.

Also provided are compositions containing N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine for treatment of an H₄ mediated condition, wherein the H₄ mediated condition is selected from the group consisting of atopic dermatitis, urticaria, psoriatic arthritis, vertigo, macular degenerative disorders, mastocytosis, inflammatory lupus erythematosus, systemic lupus erythematosus, bullous disorders, collagenoses, psoriatic lesions, seborrheic dermatitis or contact dermatitis, eczema, pruritus, uraemic pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, neuropathic pain, tinnitus, uveitis, diabetic nephropathy and multiple sclerosis.

Likewise, also provided are methods of treating an H₄ mediated condition containing administering an effective amount of a composition containing N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine to a patient in need thereof, wherein the H₄ mediated condition is selected from the group consisting of atopic dermatitis, urticaria, psoriatic arthritis, vertigo, macular degenerative disorders, mastocytosis, inflammatory lupus erythematosus, systemic lupus erythematosus, bullous disorders, collagenoses, psoriatic lesions, seborrheic dermatitis or contact dermatitis, eczema, pruritus, uraemic pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, neuropathic pain, tinnitus, uveitis, diabetic nephropathy and multiple sclerosis.

Further provided is a composition comprising N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine for use in treating an H₄ mediated condition, wherein the H₄ mediated condition is selected from the group consisting of atopic dermatitis, urticaria, uraemic pruritus, psoriatic arthritis, vertigo, macular degenerative disorders, mastocytosis, inflammatory lupus erythematosus, systemic lupus erythematosus, bullous disorders, collagenoses, psoriatic lesions, seborrheic dermatitis or contact dermatitis, eczema, pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, neuropathic pain, tinnitus, uveitis, diabetic nephropathy and multiple sclerosis.

The compositions, pharmaceutical compositions, or dosage forms can be administered to the patient via an oral, topical, intravenous, intraarterial, intraocular, intraperitoneal, intrathecal, intraventricular, intraurethral, intrasternal, intracranial, intramuscular, or subcutaneous route of administration.

For example, the compositions, pharmaceutical compositions, or dosage forms can be administered to the patient once daily.

The compositions, pharmaceutical compositions, or dosage forms can be administered at a dose of from about 1 mg to about 60 mg (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg).

In various embodiments, the compositions, pharmaceutical compositions, or dosage forms can be administered at a dose of from about 10 to about 60 mg; at a dose of from about 5 mg to about 50 mg; at a dose of from about 1 mg to about 10 mg; at a dose of from about 3 mg to about 15 mg; at a dose of from about 5 mg to about 20 mg; and/or at a dose of from about 10 mg to about 30 mg.

Any of the compositions, pharmaceutical compositions, or dosage forms can be administered intravenously, subcutaneously, or intraocularly, at a dosage of from about 0.005 to about 100 mg/ml (e.g., about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml).

In various embodiments, the compositions, pharmaceutical compositions, or dosage forms can be administered at a dose of from about 0.05 to about 100 mg/ml; at a dose of from about 0.01 to about 90 mg/ml; at a dose of from about 0.005 to about 10 mg/ml; at a dose of from about 0.05 to about 15 mg/ml; at a dose of from about 0.5 to about 20 mg/ml; at a dose of from about 10 to about 30 mg/ml.

Also provided are methods of producing N⁴-(cyclopropylmethyl)-6-[(3R)-3-(methylamino)pyrrolidin-1-yl]pyrimidine-2,4-diamine tartrate dihydrate by: a) crystallizing N⁴-(cyclopropylmethyl)-6-[(3R)-3-(methylamino)pyrrolidin-1-yl]pyrimidine 2,4-diamine tartrate from an aqueous solution of N⁴-(cyclopropylmethyl)-6-[(3R)-3-(methylamino)pyrrolidin-1-yl]pyrimidine-2,4-diamine tartrate; b) isolating the crystallized material; c) drying the isolated material under wet inert gas flow until such time that the water content of the isolated material is between 6 and 10% and any organic solvent present comprises <0.5% of the isolated material; wherein the isolated material comprises N⁴-(cyclopropylmethyl)-6-[(3R)-3-(methylamino)pyrrolidin-1-yl]pyrimidine-2,4-diamine tartrate dihydrate.

In some embodiments, the isolated material contains a polymorph of N⁴-(cyclopropylmethyl)-6-[(3R)-3-(methylamino)pyrrolidin-1-yl]pyrimidine-2,4-diamine tartrate dihydrate. For example, the polymorph is distinguished by PXRD peaks at about 6.7, 9.2, 22.4, and 24.4 degrees 2-theta. In additional embodiments, the polymorph can be identified by two additional peaks at about 13.5 and 18.7 degrees 2-theta. In further embodiments, the polymorph can be identified by four additional peaks at about 20.9, 21.4, 26.8, and 30.0 degrees 2-theta.

In these methods, the aqueous solution is treated with an organic solvent (e.g., an alcohol such as methanol).

In some embodiments, the inert gas is nitrogen.

In various embodiments, the relative water humidity in the drying chamber is more than about 40% RH; between about 50 and 99% RH; between about 60 and about 80% RH; and/or between about 69 and 99% RH.

In some embodiments of this method, N⁴-(cyclopropylmethyl)-6-[(3R)-3-(methylamino)pyrrolidin-1-yl]pyrimidine-2,4-diamine tartrate or a polymorph of N⁴-(cyclopropylmethyl)-6-[(3R)-3-(methylamino)pyrrolidin-1-yl]pyrimidine-2,4-diamine tartrate is crystallized by progressively cooling the aqueous solution of N⁴-(cyclopropylmethyl)-6-[(3R)-3-(methylamino)pyrrolidin-1-yl]pyrimidine-2,4-diamine tartrate.

For example, this may additionally involve the steps of: a) adding an amount of N⁴-(cyclopropylmethyl)-6-[(3R)-3-(methylamino)pyrrolidin-1-yl]pyrimidine-2,4-diamine (2R,3R)-tartrate to a volume of purified water to produce a first solution and warming to a temperature above 50° C.; b) charging the first solution with an organic solvent (e.g., an alcohol such as methanol) to produce a second solution; c) cooling the second solution to 40-60° C. to produce a slurry; d) progressively cooling the slurry to 20-35° C.; e) isolating the slurry; f) washing the isolated material; and g) drying the isolated material under wet inert gas (e.g., nitrogen) flow until such time that the water content of the isolated material is between 6 and 10% and any organic solvent present comprises <0.5% of the isolated material, wherein the isolated material comprises a polymorph of N⁴-(cyclopropylmethyl)-6-[(3R)-3-(methylamino)pyrrolidin-1-yl]pyrimidine-2,4-diamine tartrate dihydrate. In various embodiments, the relative water humidity in the drying chamber is more than about 40% RH; between about 50 and 99% RH; between about 60 and about 80% RH; and/or between about 69 and 99% RH.

For example, step a) can be performed at a temperature range of about 55° C. to about 65° C. and/or step c), the solution is cooled to about 50° C. over a period of about 20 to about 60 minutes. Moreover, the solution can be subsequently cooled to about 40° C. over a period of about 20 to about 60 minutes and/or subsequently cooled to about 30° C. over a period of 20 to 60 minutes.

Those skilled in the art will recognize that in these methods, the organic solvent content of the isolated material can be determined using nuclear magnetic resonance (NMR) or gas chromatography (GC).

Also provided are compositions containing a pharmaceutically or veterinarily acceptable salt of N⁴-(cyclopropylmethyl)-6-[(3R)-3-(methylamino)pyrrolidin-1-yl]pyrimidine-2,4-diamine, wherein the pharmaceutically or veterinarily acceptable salt is selected from the gentisate (gentisylate) salt, the salicylate salt, the di-hydrochloride salt, and the ethane disulfonate salt.

Further provided are compositions comprising a pharmaceutically or veterinarily acceptable salt of N⁴-(cyclopropylmethyl)-6-[(3R)-3-(methylamino)pyrrolidin-1-yl]pyrimidine-2,4-diamine, wherein the pharmaceutically or veterinarily acceptable salt is selected from the group consisting of the gentisate salt, the salicylate salt, the di-hydrochloride salt, and the ethane disulfonate salt, for use in a treating an H₄ mediated condition.

Also provided is N⁴-(cyclopropylmethyl)-6-[(3R)-3-(methylamino)pyrrolidin-1-yl]pyrimidine-2,4-diamine, or a pharmaceutically acceptable salt, solvate, or hydrate thereof, for use in treating atopic dermatitis in a patient, wherein 30 mg, 30 mg or less, 15 mg to 30 mg, 5 mg to 15 mg, or 1 mg to 5 mg, of the N⁴-(cyclopropylmethyl)-6-[(3R)-3-(methylamino)pyrrolidin-1-yl]pyrimidine-2,4-diamine, or a pharmaceutically acceptable salt, solvate, or hydrate thereof is administered to the patient once daily.

For example, in one embodiment, the composition contains N⁴-(cyclopropylmethyl)-6-[(3R)-3-(methylamino)pyrrolidin-1-yl]pyrimidine-2,4-diamine gentisate. In another embodiment, the composition contains N⁴-(cyclopropylmethyl)-6-[(3R)-3-(methylamino)pyrrolidin-1-yl]pyrimidine-2,4-diamine salicylate. In a further embodiment, the composition contains N⁴-(cyclopropylmethyl)-6-[(3R)-3-(methylamino)pyrrolidin-1-yl]pyrimidine-2,4-diamine di-hydrochloride hydrate. In a still further embodiment, the composition contains N⁴-(cyclopropylmethyl)-6-[(3R)-3-(methylamino)pyrrolidin-1-yl]pyrimidine-2,4-diamine ethane disulfonate hydrate.

Also provided are pharmaceutical compositions containing any one of compositions and a pharmaceutically acceptable carrier or diluent.

Likewise, dosage forms containing an effective amount of any of these compositions or pharmaceutical compositions are also contemplated. By way of non-limiting example, the dosage form can be powder-in-capsule forms, capsules, tablets, liquids, powders, lozenges, chews, multi- and nano-particulates, gels, solid solutions, liposomes, nanoparticles, films, ovules, sprays, injectables, and liquid formulations.

Also provided are compositions, pharmaceutical compositions, or dosage forms for treatment of an H₄ mediated disease or condition as well as methods of treating an H₄ mediated disease or condition by administering an effective amount of any of the compositions, pharmaceutical compositions, and/or dosage forms described herein to a patient in need thereof.

By way of non-limiting example, the H₄ mediated disease or condition is selected from the group consisting of inflammatory skin diseases (i.e., atopic dermatitis or psoriasis), pruritic diseases (i.e., urticaria or uraemic pruritus), respiratory diseases (i.e., asthma, chronic obstructive airway disease, or allergic rhinitis), cardiac diseases (i.e., myocardial ischaemia), inflammatory diseases of the gastrointestinal tract (i.e., Crohn's disease or colitis ulcerosa), cancer, joint diseases (i.e., rheumatoid arthritis or psoriatic arthritis), kidney diseases (i.e., diabetic nephropathy), pain disorders (i.e., inflammatory pain or neuropathic pain), overactive bladder conditions, vestibular disorders (i.e., vertigo or tinnitus), macular degenerative disorders, inflammatory eye diseases (i.e., conjunctivitis or uveitis), and other diseases involving immune and inflammatory disorders (i.e., multiple sclerosis, mastocytosis, or inflammatory or systemic lupus erythematosus).

In some embodiments, the H₄ mediated disease or condition is selected from the group consisting of atopic dermatitis, bullous disorders, collagenoses, psoriasis, psoriatic lesions, seborrheic dermatitis or contact dermatitis, eczema, urticaria, pruritus, uraemic pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, squamous cell carcinoma or melanoma.

In some preferred embodiments, the disease or condition is psoriasis, atopic dermatitis, or other pruritic conditions.

The compositions, pharmaceutical compositions, or dosage forms can be administered to the patient via an oral, topical, intravenous, intraarterial, intraocular, intraperitoneal, intrathecal, intraventricular, intraurethral, intrasternal, intracranial, intramuscular, or subcutaneous route of administration.

For example, the compositions, pharmaceutical compositions, or dosage forms can be administered to the patient once daily.

The compositions, pharmaceutical compositions, or dosage forms can be administered at a dose of from about 1 mg to about 60 mg (e.g., about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg).

In various embodiments, the compositions, pharmaceutical compositions, or dosage forms can be administered at a dose of from about 10 to about 60 mg; at a dose of from about 5 mg to about 50 mg; at a dose of from about 1 mg to about 10 mg; at a dose of from about 3 mg to about 15 mg; at a dose of from about 5 mg to about 20 mg; and/or at a dose of from about 10 mg to about 30 mg.

Any of the compositions, pharmaceutical compositions, or dosage forms can be administered intravenously, subcutaneously, or intraocularly, at a dosage of from about 0.005 to about 100 mg/ml (e.g., about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml).

In various embodiments, the compositions, pharmaceutical compositions, or dosage forms can be administered at a dose of from about 0.05 to about 100 mg/ml; at a dose of from about 0.01 to about 90 mg/ml; at a dose of from about 0.005 to about 10 mg/ml; at a dose of from about 0.05 to about 15 mg/ml; at a dose of from about 0.5 to about 20 mg/ml; at a dose of from about 10 to about 30 mg/ml.

Any of the compositions, pharmaceutical compositions, or dosage forms can be administered to the patient with one or more additional therapeutic agents. By way of non-limiting example, the one or more additional therapeutic agents are selected from Histamine H₁ receptor antagonists (i.e., fexofenadine, cetirizine, levocetrizine, loratadine, desloratadine, mepyramine, and diphenhydramine); Histamine H₃ receptor antagonists; Histamine H₂ receptor antagonists; leukotriene antagonists (i.e., montelukast, zafirlukast, and pranlukast); phosphodiesterase inhibitors (i.e., PDE4 phosphodiesterase inhibitors such as apremilast or roflumilast); neurotransmitter re-uptake inhibitors; 5-lipooxygenase (5-LO) inhibitors; 5-lipoxygenase activating protein (FLAP) inhibitors; α₁- and α₂-adrenoceptor agonist vasoconstrictor sympathomimetic agents; muscarinic M₃ receptor antagonists or anticholinergic agents; β₂-adrenoceptor agonists; dual acting β₂/M₃ agents; xanthines; non-steroidal anti-inflammatories; ketotifen; COX-1 inhibitors (NSAIDs) and COX-2 selective inhibitors; oral, inhaled intranasal and topical glucocorticosteroids; monoclonal antibodies active against endogenous inflammatory entities; anti-tumor necrosis factor (anti-TNF-α) agents; adhesion molecule inhibitors including VLA-4 antagonists; kinin-B₁- and B₂-receptor antagonists; immunosuppressive agents; inhibitors of matrix metalloproteases (MMPs); tachykinin NK₁, NK₂ and NK₃ receptor antagonists; elastase inhibitors; adenosine A2a receptor agonists; inhibitors of urokinase; compounds that act on dopamine receptors; modulators of the NKκb pathway; agents that can be classed as mucolytics or anti-tussive agents; antibiotics; modulators of cytokine signaling pathways; modulators of the prostaglandin pathways; antagonists of chemokine receptors CXCR1 and CXCR2; antagonists of chemokine receptors CCR3, CCR4 and CCR5; inhibitors of cytosolic and soluble phospholipase A₂ (cPLA₂ and sPLA₂); inhibitors of phosphoinositide-3-kinase; HDAC inhibitors; p38 inhibitors; CXCR2 antagonists; calcineurin inhibitors; anti-interleukin 17 (anti-IL-17) agents; anti-interleukin 4 receptor (anti-IL4R) agents; anti-interleukin 31 (anti-IL-31) agents; CRTH2 antagonists (i.e., ADC3680, NVP-QAV680, and OC459); and combinations thereof.

Also provided are methods of treating atopic dermatitis in a patient by administering 30 mg or less of ZPL-389 to the patient once daily. For example, methods of treating atopic dermatitis in a patient by administering 15 mg to 30 mg of ZPL-389 to the patient once daily, or methods of treating atopic dermatitis in a patient by administering 5 mg to 15 mg of ZPL-389 to the patient once daily, or methods of treating atopic dermatitis in a patient by administering 1 mg to 5 mg of ZPL-389 to the patient once daily. Also provided are methods of treating atopic dermatitis in a patient by administering 30 mg of ZPL-389 to the patient once daily. For example, ZPL-389 can be administered orally, i.e., in a form selected from powder-in-capsule, capsule, tablet, liquid, powder, lozenge, chew, multi- and nano-particulate, gel, solid solution, liposome, nanoparticle, film, ovule, spray, and liquid formulation. In one embodiment, 30 mg of ZPL-389 is administered orally once daily to patients suffering from moderate to severe atopic dermatitis (the most common form of eczema). Following 8 weeks of treatment, patients exhibit a clinically and statistically significant decrease in inflammation compared to placebo, as evidenced, for example, by a reduction in Eczema Area and Severity Index, an improvement on SCORing Atopic Dermatitis, and an improvement in Body Surface Area.

Previously, two single doses of ZPL-389 given 12 hours apart had not been shown to be efficacious in the treatment of mild asthma. Specifically, no efficacy was observed in one human lung allergen trial where ZPL-389 was administered at 36 mg in two single doses given 12 hours apart to mild asthmatics. As a result of the failure at this dose, a person skilled in the art would have increased the amount of ZPL-389 administered to the patient in an attempt to find an efficacious oral dose. Additionally, a skilled person would also attempt to utilize a different route of administration in order to treat inflammatory conditions with ZPL-389, as oral administration of ZPL-389 was shown to be ineffective in treating asthma (another inflammatory condition).

Accordingly, it is both surprising and unexpected that ZPL-389 was found to be efficacious at a lower dose of 30 mg when administered orally once daily for the treatment of atopic dermatitis, an inflammatory condition that shares certain common pathobiology to asthma (See Example 10, infra.).

N⁴-(cyclopropylmethyl)-6-[(3R)-3-(methylamino)pyrrolidin-1-yl]pyrimidine-2,4-diamine, pharmaceutically acceptable salts thereof, and pharmaceutical compositions comprising the same may be used for treatment of H₄ mediated diseases or conditions. However, the presence of the impurity, 4-N-butyl-6-[(3-(methylamino)pyrrolidin-1-yl]pyrimidine-2,4-diamine, in any of the compositions, pharmaceutical compositions, and/or dosage forms (e.g., tablets) described herein is expected to be detrimental to the efficacy of the compositions, pharmaceutical compositions, and/or dosage forms in treating H₄ mediated diseases or conditions. The impurity, 4-N-butyl-6-[(3-(methylamino)pyrrolidin-1-yl]pyrimidine-2,4-diamine (also referred to as PF-04360799) may lead to unwanted side effects in the patient, among other negative outcomes. Specifically, this impurity may be carcinogenic and/or cause skin irritation or sensitization. Therefore, minimizing the amount of the impurity in any of the compositions, pharmaceutical compositions, and/or dosage forms (e.g., tablets) described herein is expected to be advantageous in treating H₄ mediated diseases or conditions.

N⁴-(cyclopropylmethyl)-6-[(3R)-3-(methylamino)pyrrolidin-1-yl]pyrimidine-2,4-diamine and the impurity, 4-N-butyl-6-[(3-(methylamino)pyrrolidin-1-yl]pyrimidine-2,4-diamine, are structurally similar. The only difference in their structures is a cyclopropyl methyl group in N⁴-(cyclopropylmethyl)-6-[(3R)-3-(methylamino)pyrrolidin-1-yl]pyrimidine-2,4-diamine in place of an n-butyl group in the impurity. As a result, the physicochemical properties (e.g., partition coefficient (Log P), total surface polarity (tPSA), boiling point, melting point, pKa, etc.) of N⁴-(cyclopropylmethyl)-6-[(3R)-3-(methylamino)pyrrolidin-1-yl]pyrimidine-2,4-diamine and the impurity, 4-N-butyl-6-[(3-(methylamino)pyrrolidin-1-yl]pyrimidine-2,4-diamine are similar.

Due to the similarities in their physicochemical properties, it would be impractical to separate N⁴-(cyclopropylmethyl)-6-[(3R)-3-(methylamino)pyrrolidin-1-yl]pyrimidine-2,4-diamine and the impurity, 4-N-butyl-6-[(3-(methylamino)pyrrolidin-1-yl]pyrimidine-2,4-diamine, using traditional means such as HPLC or column chromatography.

Thus, the development of ultra-pure compositions of N⁴-(cyclopropylmethyl)-6-[(3R)-3-(methylamino)pyrrolidin-1-yl]pyrimidine-2,4-diamine, and salts thereof, is critical to developing reliable methods for treating H₄ mediated diseases or conditions.

The use of wet inert gas during the purification/drying stages in the preparation of compositions containing pharmaceutically acceptable salts of N⁴-(cyclopropylmethyl)-6-[(3R)-3-(methylamino)pyrrolidin-1-yl]pyrimidine-2,4-diamine has been found to be a reliable method for the preparation of ultra-pure compositions of N⁴-(cyclopropylmethyl)-6-[(3R)-3-(methylamino)pyrrolidin-1-yl]pyrimidine-2,4-diamine. For example, the use of wet inert gas has been found to be a reliable method for the preparation of ultra-pure compositions containing N⁴-(cyclopropylmethyl)-6-[(3R)-3-(methylamino)pyrrolidin-1-yl]pyrimidine-2,4-diamine tartrate dihydrate.

Also provided herein are tablets containing a therapeutically effective amount of N⁴-(cyclopropylmethyl)-6-[(3R)-3-(methylamino)pyrrolidin-1-yl]pyrimidine-2,4-diamine and one or more pharmaceutically acceptable carriers, diluents or excipients.

In some embodiments, the therapeutically effective amount is 1 to 100 mg, 1 to 60 mg, or 30 mg.

In other embodiments, N⁴-(cyclopropylmethyl)-6-[(3R)-3-(methylamino)pyrrolidin-1-yl]pyrimidine-2,4-diamine is in the form of N⁴-(cyclopropylmethyl)-6-[(3R)-3-(methylamino)pyrrolidin-1-yl]pyrimidine-2,4-diamine tartrate dihydrate.

In various embodiments, the therapeutically effective amount of N⁴-(cyclopropylmethyl)-6-[(3R)-3-(methylamino)pyrrolidin-1-yl]pyrimidine-2,4-diamine tartrate dihydrate in the tablets is between 1 and 175 mg. For example, 1 to about 110 mg, about 52 mg, about 17.2 mg, about 5.2 mg, or about 1.7 mg. In other examples, 1 to 110 mg, 52 mg, 17.2 mg, 5.2 mg, or 1.7 mg. See Examples 11-14, infra. Any suitable method known in the art can be used to formulate the tablets.

In some embodiments, the tablets are prepared by a dry granulation formulation method.

In other embodiments, the tablets are prepared by a wet granulation formulation method, a direct compression formulation method, or a moisture activated dry granulation formulation method.

By way of non-limiting example, the tablets may further contain one or more additional ingredients, such as microcrystalline cellulose (MCC), mannitol, croscarmellose sodium, sodium starch glycolate, dicalcium phosphate anhydrous (DCP), hydroxypropyl cellulose (HPC), povidone, crospovidone, silicon dioxide, magnesium stearate, and/or any other excipients known in the art.

Also provided herein is a tablet (e.g., a tablet prepared by a dry formulation method) containing:

(a) about 25.75% by weight of N⁴-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose; and

(c) about 17.85% by weight of dicalcium phosphate anhydrous.

Such tablets may additionally contain sodium starch glycolate, croscarmellose sodium, and/or magnesium stearate.

Any of the excipients used herein may be included as intra-granular excipients, extra-granular excipients, or a combination thereof. For example, without limitation, microcrystalline cellulose, dicalcium phosphate anhydrous, sodium starch glycolate, croscarmellose sodium, and/or magnesium stearate, may be included as intra-granular excipients, extra-granular excipients, or a combination thereof.

Also provided herein is a tablet (e.g., a tablet prepared by a dry granulation method) containing:

(a) about 25.75% by weight of N4-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose;

(c) about 17.85% by weight of dicalcium phosphate anhydrous; and

(d) about 8% by weight of croscarmellose sodium.

Also provided herein is a tablet (e.g., a tablet prepared by a dry granulation method) containing:

(a) about 25.75% by weight of N4-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose;

(c) about 17.85% by weight of dicalcium phosphate anhydrous;

(d) about 8% by weight of croscarmellose sodium; and

(e) about 1% by weight of a lubricant.

Also provided herein are tablets comprising a therapeutically effective amount of N⁴-(cyclopropylmethyl)-6-[(3R)-3-(methylamino)pyrrolidin-1-yl]pyrimidine-2,4-diamine tartrate dihydrate and one or more pharmaceutically acceptable carriers, diluents or excipients of the invention, for use in treating atopic dermatitis in a patient, wherein the tablet is administered to the patient once daily.

Also provided herein is a tablet (e.g., a tablet prepared by a dry granulation method) containing:

(a) about 25.75% by weight of N4-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose;

(c) about 17.85% by weight of dicalcium phosphate anhydrous;

(d) about 8% by weight of croscarmellose sodium; and

(e) about 1% by weight of magnesium stearate.

Also provided herein is a tablet (e.g., a tablet prepared by a wet granulation method) containing:

(a) about 51.5% by weight of N⁴-(cyclopropylmethyl)-6-[(3R)-3-(methylamino)pyrrolidin-1-yl]pyrimidine-2,4-diamine tartrate dihydrate;

(b) about 19.75% by weight of microcrystalline cellulose; and

(c) about 19.75% by weight of dicalcium phosphate anhydrous.

Such tablets may additionally contain sodium starch glycolate, hydroxypropyl cellulose, and/or magnesium stearate.

Any of the excipients used herein may be included as intra-granular excipients, extra-granular excipients, or a combination thereof. For example, without limitation, microcrystalline cellulose, dicalcium phosphate anhydrous, sodium starch glycolate, hydroxypropyl cellulose, and/or magnesium stearate, may be included as intra-granular excipients, extra-granular excipients, or a combination thereof.

Also provided are methods of treating atopic dermatitis in a patient, by administering a tablet comprising a therapeutically effective amount of N⁴-(cyclopropylmethyl)-6-[(3R)-3-(methylamino)pyrrolidin-1-yl]pyrimidine-2,4-diamine tartrate dihydrate and one or more pharmaceutically acceptable carriers, diluents or excipients, to the patient once daily.

By way of non-limiting example, the therapeutically effective amount of N⁴-(cyclopropylmethyl)-6-[(3R)-3-(methylamino)pyrrolidin-1-yl]pyrimidine-2,4-diamine is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg.

By way of non-limiting example, the tablets described herein may contain 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 69, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100, 100.5, 101, 101.5, 102, 102.5, 103, 103.5, 104, 104.5, 105, 105.5, 106, 106.5, 107, 107.5, 108, 108.5, 109, 109.5, 110, 110.5, 111, 111.5, 112, 112.5, 113, 113.5, 114, 114.5, 115, 115.5, 116, 116.5, 117, 117.5, 118, 118.5, 119, 119.5, 120, 120.5, 121, 121.5, 122, 122.5, 123, 123.5, 124, 124.5, 125, 125.5, 126, 126.5, 127, 127.5, 128, 128.5, 129, 129.5, 130, 130.5, 131, 131.5, 132, 132.5, 133, 133.5, 134, 134.5, 135, 135.5, 136, 136.5, 137, 137.5, 138, 138.5, 139, 139.5, 140, 140.5, 141, 141.5, 142, 142.5, 143, 143.5, 144, 144.5, 145, 145.5, 146, 146.5, 147, 147.5, 148, 148.5, 149, 149.5, 150, 150.5, 151, 151.5, 152, 152.5, 153, 153.5, 154, 154.5, 155, 155.5, 156, 156.5, 157, 157.5, 158, 158.5, 159, 159.5, 160, 160.5, 161, 161.5, 162, 162.5, 163, 163.5, 164, 164.5, 165, 165.5, 166, 166.5, 167, 167.5, 168, 168.5, 169, 169.5, 170, 170.5, 171, 171.5, 172, 172.5, 173, 173.5, 174, 174.5, or 175 mg of N⁴-(cyclopropylmethyl)-6-[(3R)-3-(methylamino)pyrrolidin-1-yl]pyrimidine-2,4-diamine tartrate dihydrate.

Any of the tablets described herein may contain about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% by weight of N⁴-(cyclopropylmethyl)-6-[(3R)-3-(methylamino)pyrrolidin-1-yl]pyrimidine-2,4-diamine tartrate dihydrate.

Any of the tablets described herein may contain about 0, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% by weight of microcrystalline cellulose.

Any of the tablets described herein may contain about 0, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% by weight of dicalcium phosphate anhydrous.

Unless otherwise defined, all technical and scientific terms used herein have the same meaning as commonly understood by one of ordinary skill in the art to which this application belongs. In the specification, the singular forms also include the plural unless the context clearly dictates otherwise.

Although methods and materials similar to or equivalent to those described herein can be used in the practice and testing of the application, suitable methods and materials are described below. All publications, patent applications, patents, and other references mentioned herein are incorporated by reference.

The references cited herein are not admitted to be prior art to the claimed application. In the case of conflict, the present specification, including definitions, will control. In addition, the materials, methods, and examples are illustrative only and not intended to be limiting.

Other features and advantages of the application will become apparent from the following detailed description in conjunction with the examples.

### BRIEF DESCRIPTION OF THE FIGURES

FIG. 1 shows PXRD of N⁴-(cyclopropylmethyl)-6-[(3R)-3-(methylamino)pyrrolidin-1-yl]pyrimidine-2,4-diamine tartrate dihydrate (Form A).

FIG. 2 shows the peak listing of PXRD of N⁴-(cyclopropylmethyl)-6-[(3R)-3-(methylamino)pyrrolidin-1-yl]pyrimidine-2,4-diamine tartrate dihydrate (Form A).

FIG. 3 shows the ¹H NMR of ultra-pure N⁴-(cyclopropylmethyl)-6-[(3R)-3-(methylamino)pyrrolidin-1-yl]pyrimidine-2,4-diamine tartrate dihydrate (Form A) in DMSO-d₆.

FIG. 4 shows the IR spectrum of ultra-pure N⁴-(cyclopropylmethyl)-6-[(3R)-3-(methylamino)pyrrolidin-1-yl]pyrimidine-2,4-diamine tartrate dihydrate (Form A).

FIG. 5 shows a DSC Thermogram of ultra-pure N⁴-(cyclopropylmethyl)-6-[(3R)-3-(methylamino)pyrrolidin-1-yl]pyrimidine-2,4-diamine tartrate dihydrate (Form A).

FIG. 6 shows a TGA/SDTA Thermogram of ultra-pure N⁴-(cyclopropylmethyl)-6-[(3R)-3-(methylamino)pyrrolidin-1-yl]pyrimidine-2,4-diamine tartrate dihydrate (Form A).

FIG. 7 shows a TGA/MS Thermogram of ultra-pure N⁴-(cyclopropylmethyl)-6-[(3R)-3-(methylamino)pyrrolidin-1-yl]pyrimidine-2,4-diamine tartrate dihydrate (Form A).

FIG. 8 shows an analysis of purity by LCMS of ultra-pure N⁴-(cyclopropylmethyl)-6-[(3R)-3-(methylamino)pyrrolidin-1-yl]pyrimidine-2,4-diamine tartrate dihydrate (Form A).

FIG. 9 shows a DVS analysis of ultra-pure N⁴-(cyclopropylmethyl)-6-[(3R)-3-(methylamino)pyrrolidin-1-yl]pyrimidine-2,4-diamine tartrate dihydrate (Form A).

FIG. 10 shows PXRD of N⁴-(cyclopropylmethyl)-6-[(3R)-3-(methylamino)pyrrolidin-1-yl]pyrimidine-2,4-diamine gentisate.

FIG. 11 shows the ¹H NMR of N⁴-(cyclopropylmethyl)-6-[(3R)-3-(methylamino)pyrrolidin-1-yl]pyrimidine-2,4-diamine gentisate in DMSO-d₆.

FIG. 12 shows a TGA analysis of N⁴-(cyclopropylmethyl)-6-[(3R)-3-(methylamino)pyrrolidin-1-yl]pyrimidine-2,4-diamine gentisate.

FIG. 13 shows a TGMS analysis of N⁴-(cyclopropylmethyl)-6-[(3R)-3-(methylamino)pyrrolidin-1-yl]pyrimidine-2,4-diamine gentisate.

FIG. 14 shows PXRD of N⁴-(cyclopropylmethyl)-6-[(3R)-3-(methylamino)pyrrolidin-1-yl]pyrimidine-2,4-diamine salicylate.

FIG. 15 shows the peak listing of PXRD of N⁴-(cyclopropylmethyl)-6-[(3R)-3-(methylamino)pyrrolidin-1-yl]pyrimidine-2,4-diamine salicylate.

FIG. 16 shows the ¹H NMR of N⁴-(cyclopropylmethyl)-6-[(3R)-3-(methylamino)pyrrolidin-1-yl]pyrimidine-2,4-diamine salicylate in DMSO-d₆.

FIG. 17 shows the IR spectrum of N⁴-(cyclopropylmethyl)-6-[(3R)-3-(methylamino)pyrrolidin-1-yl]pyrimidine-2,4-diamine salicylate.

FIG. 18 shows a TGA analysis of N⁴-(cyclopropylmethyl)-6-[(3R)-3-(methylamino)pyrrolidin-1-yl]pyrimidine-2,4-diamine salicylate.

FIG. 19 shows a TGMS analysis of N⁴-(cyclopropylmethyl)-6-[(3R)-3-(methylamino)pyrrolidin-1-yl]pyrimidine-2,4-diamine salicylate.

FIG. 20 shows PXRD of N⁴-(cyclopropylmethyl)-6-[(3R)-3-(methylamino)pyrrolidin-1-yl]pyrimidine-2,4-diamine di-hydrochloride hydrate.

FIG. 21 shows the peak listing of PXRD of N⁴-(cyclopropylmethyl)-6-[(3R)-3-(methylamino)pyrrolidin-1-yl]pyrimidine-2,4-diamine di-hydrochloride hydrate.

FIG. 22 shows the ¹H NMR of N⁴-(cyclopropylmethyl)-6-[(3R)-3-(methylamino)pyrrolidin-1-yl]pyrimidine-2,4-diamine di-hydrochloride hydrate in DMSO-d₆.

FIG. 23 shows the IR spectrum of N⁴-(cyclopropylmethyl)-6-[(3R)-3-(methylamino)pyrrolidin-1-yl]pyrimidine-2,4-diamine di-hydrochloride hydrate.

FIG. 24 shows a TGS/SDTA analysis of N⁴-(cyclopropylmethyl)-6-[(3R)-3-(methylamino)pyrrolidin-1-yl]pyrimidine-2,4-diamine di-hydrochloride hydrate.

FIG. 25 shows a TGMS analysis of N⁴-(cyclopropylmethyl)-6-[(3R)-3-(methylamino)pyrrolidin-1-yl]pyrimidine-2,4-diamine di-hydrochloride hydrate.

FIG. 26 shows PXRD of N⁴-(cyclopropylmethyl)-6-[(3R)-3-(methylamino)pyrrolidin-1-yl]pyrimidine-2,4-diamine ethane disulfonate hydrate.

FIG. 27 shows the peak listing of PXRD of N⁴-(cyclopropylmethyl)-6-[(3R)-3-(methylamino)pyrrolidin-1-yl]pyrimidine-2,4-diamine ethane disulfonate hydrate.

FIG. 28 shows the ¹H NMR of N⁴-(cyclopropylmethyl)-6-[(3R)-3-(methylamino)pyrrolidin-1-yl]pyrimidine-2,4-diamine ethane disulfonate hydrate in DMSO-d₆.

FIG. 29 shows the IR spectrum of N⁴-(cyclopropylmethyl)-6-[(3R)-3-(methylamino)pyrrolidin-1-yl]pyrimidine-2,4-diamine ethane disulfonate hydrate.

FIG. 30 shows a TGA/SDTA analysis of N⁴-(cyclopropylmethyl)-6-[(3R)-3-(methylamino)pyrrolidin-1-yl]pyrimidine-2,4-diamine ethane disulfonate hydrate.

FIG. 31 shows a TGMS analysis of N⁴-(cyclopropylmethyl)-6-[(3R)-3-(methylamino)pyrrolidin-1-yl]pyrimidine-2,4-diamine ethane disulfonate hydrate.

FIG. 32 shows a comparison of the results of dissolution experiments of the modified 30 mg dry and wet granulation formulation tablets (see Examples 15 and 16, infra) in 0.01 M HCl.

FIG. 33 shows a comparison of the results of dissolution experiments of the modified 30 mg dry and wet granulation formulation tablets (see Examples 15 and 16, infra) in pH 6.8 buffer.

### DETAILED DESCRIPTION OF THE INVENTION

As one of skill in the art would appreciate, the PXRD peaks for any polymorph or a composition containing a polymorph may vary based on the experimental conditions and/or skill/experience level of the operator of the instrument. As used herein, the term “about,” when used in the context of reciting PXRD peaks for a polymorph or a composition containing a polymorph, refers to the recited peak +/−0.2 degrees two theta.

As used herein in other contexts, the term “about,” unless indicated otherwise, refers to the recited value, e.g., amount, dose, temperature, time, percentage, etc., +/−10%, +/−9%, +/−8%, +/−7%, +/−6%, +/−5%, +/−4%, +/−3%, +/−2%, or +/−1%.

As used herein, the phrase “wet inert gas” refers to an inert gas that has a relative water humidity of greater than about 40%, i.e., >40% RH. For example, the wet inert gas may be about 45% to about 99% RH, about 50% to about 99% RH, about 55% to about 99% RH, about 60% to about 99% RH, about 65% to about 99% RH, about 66% to about 99% RH, about 67% to about 99% RH, about 68% to about 99% RH, about 69% to about 99% RH, about 70% to about 99% RH, about 71% to about 99% RH, about 72% to about 99% RH, about 73% to about 99% RH, about 74% to about 99% RH, about 75% to about 99% RH, about 80% to about 99% RH, about 85% to about 99% RH, about 90% to about 99% RH, about 75% to about 99% RH, about 80% to about 99% RH. In other embodiments, the wet inert gas is between about 40% RH and about 60% RH, about 45% RH and about 65% RH, about 50% RH and about 70% RH, about 55% RH and about 75% RH, about 60% RH and about 80% RH, about 65% RH and about 85% RH, about 70% RH and about 90% RH, about 75% RH and about 95% RH, about 88% RH and 99% RH.

For example, the wet inert gas can be provided by introducing water into the apparatus that contains the composition being purified. For example, the composition to be purified under inert gas flow can be placed in a vacuum oven along with a container containing water.

For example, the inert gas is nitrogen or argon. In one preferred embodiment, the inert gas is nitrogen. In still yet another embodiment, the inert gas is nitrogen that has a relative water humidity of greater than about 40%, for example, 41% RH, 42% RH, 43% RH, 44% RH, 45% RH, 46% RH, 47% RH, 48% RH, 49% RH, 50% RH, 51% RH, 52% RH, 53% RH, 54% RH, 55% RH, 56% RH, 57% RH, 58% RH, 59% RH, 60% RH, 61% RH, 62% RH, 63% RH, 64% RH, 65% RH, 66% RH, 67% RH, 68% RH, 69% RH, 70% RH, 71% RH, 72% RH, 73% RH, 74% RH, 75% RH, 76% RH, 77% RH, 78% RH, 79% RH, 80% RH, 81% RH, 82% RH, 83% RH, 84% RH, 85% RH, 86% RH, 87% RH, 88% RH, 89% RH, 90% RH, 91% RH, 92% RH, 93% RH, 94% RH, 95% RH, 96% RH, 97% RH, 98% RH, or 99% RH.

As used herein, the term “ultra-pure,” as it pertains to “ultra-pure N⁴-(cyclopropylmethyl)-6-[(3R)-3-(methylamino)pyrrolidin-1-yl]pyrimidine-2,4-diamine tartrate dihydrate (Form A),” and other compositions containing N⁴-(cyclopropylmethyl)-6-[(3R)-3-(methylamino)pyrrolidin-1-yl]pyrimidine-2,4-diamine, or salts/solvates (e.g., hydrates) thereof and the like, refers to a highly pure form of the compound and/or salt/solvate thereof. For example, the ultra-pure form of N⁴-(cyclopropylmethyl)-6-[(3R)-3-(methylamino)pyrrolidin-1-yl]pyrimidine-2,4-diamine, N⁴-(cyclopropylmethyl)-6-[(3R)-3-(methylamino)pyrrolidin-1-yl]pyrimidine-2,4-diamine tartrate dihydrate, or any other salt/solvate thereof, is greater than 98% pure, greater than 99% pure, greater than 99.5% pure, greater than 99.6% pure, greater than 99.7% pure, greater than 99.8% pure, or greater than 99.9% pure. In certain embodiments, the ultra-pure form of N⁴-(cyclopropylmethyl)-6-[(3R)-3-(methylamino)pyrrolidin-1-yl]pyrimidine-2,4-diamine, N⁴-(cyclopropylmethyl)-6-[(3R)-3-(methylamino)pyrrolidin-1-yl]pyrimidine-2,4-diamine tartrate dihydrate, or any other salt/solvate thereof, contains less than 1% (i.e., less than 0.95%, 0.90%, 0.85%, 0.80%, 0.75%, 0.70%, 0.65%, 0.60%, 0.55%, 0.50%, 0.45%, 0.40%, 0.35%, 0.30%, 0.29%, 0.28%, 0.27%, 0.26%, 0.25%, 0.24%, 0.23%, 0.22%, 0.21%, 0.20%, 0.15%, 0.10%, or 0.05%) of an impurity. In one non-limiting example, an ultra-pure form of N⁴-(cyclopropylmethyl)-6-[(3R)-3-(methylamino)pyrrolidin-1-yl]pyrimidine-2,4-diamine or N⁴-(cyclopropylmethyl)-6-[(3R)-3-(methylamino)pyrrolidin-1-yl]pyrimidine-2,4-diamine tartrate dihydrate contains less than 1% (i.e., less than 0.95%, 0.90%, 0.85%, 0.80%, 0.75%, 0.70%, 0.65%, 0.60%, 0.55%, 0.50%, 0.45%, 0.40%, 0.35%, 0.30%, 0.29%, 0.28%, 0.27%, 0.26%, 0.25%, 0.24%, 0.23%, 0.22%, 0.21%, 0.20%, 0.15%, 0.10%, or 0.05%) of 4-N-butyl-6-[(3-(methylamino)pyrrolidin-1-yl]pyrimidine-2,4-diamine i.e.,

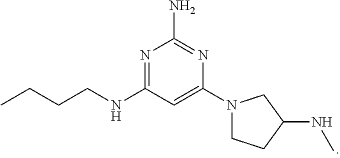 [SMILES: CCCCNc1cc(N2CCC(NC)C2)nc(N)n1]

In another example, the ultra-pure form of N⁴-(cyclopropylmethyl)-6-[(3R)-3-(methylamino)pyrrolidin-1-yl]pyrimidine-2,4-diamine and/or salt/solvate thereof is ultra-pure N⁴-(cyclopropylmethyl)-6-[(3R)-3-(methylamino)pyrrolidin-1-yl]pyrimidine-2,4-diamine tartrate dihydrate (Form A). As referred to herein, ultra-pure forms of N⁴-(cyclopropylmethyl)-6-[(3R)-3-(methylamino)pyrrolidin-1-yl]pyrimidine-2,4-diamine (e.g., N⁴-(cyclopropylmethyl)-6-[(3R)-3-(methylamino)pyrrolidin-1-yl]pyrimidine-2,4-diamine tartrate dihydrate) have a higher degree of purity and/or include a lower amount of an impurity compared to the compounds described in U.S. Pat. No. 7,943,628. Specifically, N⁴-(cyclopropylmethyl)-6-[(3R)-3-(methylamino)pyrrolidin-1-yl]pyrimidine-2,4-diamine tartrate dihydrate made by the method described in U.S. Pat. No. 7,943,628 had purity (measured by LCMS) of 95.4% and 96.1% assay.

As used herein, the term “patient” refers to a living being that includes, without limitation, rodents, dogs, cattle, sheep, and primates. In one preferred embodiment, the term “patient” refers to a human.

The term “treating” and “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. Preferably, treatment is curative or ameliorating.

Clinical efficacy can be assessed using the Eczema Area and Severity Index (EAST). An EASI score is a tool used to measure the extent (area) and severity of atopic eczema, and is composed of an area score and a severity score.

The area score is recorded for each of the four regions of the body (head and neck, trunk, upper limbs, lower limbs) and is the percentage of skin affected by eczema.

The severity score is recorded for each of the four regions of the body and is the sum of the intensity scores for four signs: redness (erythema, inflammation), thickness (induration, papulation, swelling), scratching (excoriation), lichenification (lined skin, prurigo nodules). The average intensity of each sign in each body region is assessed as: none (0), mild (1), moderate (2) and severe (3).

For each region, the severity score is multiplied by the area score and by a multiplier that is different for each body site: head and neck—severity score×area score×0.1 (in children 0-7 years, ×0.2); trunk—severity score×area score×0.3; upper limbs—severity score×area score×0.2; lower limbs—severity score×area score×0.4 (in children 0-7 years, ×0.3). The total scores for each region are added to determine the final EASI score. The minimum EASI score is 0 and the maximum EASI score is 72. (See Hanifin J. M. et al. Exp. Dermatol. 2001, 10(1):11-8).

Clinical efficacy can also be assessed using the Investigator Global Assessment (IGA), which is the physician's overall or global assessment of the condition and accounts for admixture lesion types. IGA is a static evaluation (no reference to baseline) of the overall severity of atopic dermatitis at a given time. It assesses overall disease severity at one given time point (clear, almost clear, mild disease, moderate disease, severe disease). It uses clinical characteristics such as erythema, infiltration, papulation and oozing/crusting. It allows rapid overall evaluation of the disease severity.

Alternatively (or additionally), clinical efficacy can be assessed using the SCORing Atopic Dermatitis (SCORAD), which is a clinical tool used to assess the extent and severity of eczema. Dermatologists may use this tool before and after treatment to determine whether the treatment has been effective.

To determine extent, the sites affected by eczema are shaded on a drawing of a body. The rule of 9 is used to calculate the affected area (A) as a percentage of the whole body (i.e., head and neck 9%, upper limbs 9% each, lower limbs 18% each, anterior trunk 18%, back 18%, 1% for genitals), and the score for each area is added up. The total area is ‘A’, which has a possible maximum of 100%. A representative area of eczema is then selected, and the intensity of redness, swelling oozing/crusting, scratch marks, skin thickening (lichenification) is assessed as none (0), mild (1), moderate (2) or severe (3). The intensity scores are added together to give ‘B’ (maximum 18). Subjective symptoms (i.e., itch and sleeplessness) are each scored by the patient or relative using a visual analogue scale where 0 is no itch (or no sleeplessness) and 10 is the worst imaginable itch (or sleeplessness). These scores are added to give ‘C’ (maximum 20). The total score for that individual is then calculated as A/5+7B/2+C. (See Dermatology 1993, 186:23-31).

The phrase “Carr's Index,” as used herein, is an indication of the compressibility and flow behavior of a powder.

The phrase, “therapeutically effective amount” as used herein indicates an amount necessary to administer to a host, or to a cell, tissue, or organ of a host, to achieve a therapeutic effect, such as an ameliorating or alternatively a curative effect.

The term “cancer” refers to any cancer caused by the proliferation of neoplastic cells, such as solid tumors, neoplasms, carcinomas, sarcomas, leukemias, lymphomas and the like. In particular, cancers that may be treated by the compounds, compositions and methods of the application include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma, (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor, nephroblastoma, lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcorna, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma, serous cystadenocarcinoma, mucinous cystadenocarcinoma, unclassified carcinoma, granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (acute myeloid leukemia, chronic myeloid leukemia, acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma.

The terms “tartaric acid” and “tartrate” refer to L-tartaric acid and the conjugate base thereof, unless indicated otherwise.

The present application relates to compositions containing N⁴-(cyclopropylmethyl)-6-[(3R)-3-(methylamino)pyrrolidin-1-yl]pyrimidine-2,4-diamine tartrate dihydrate, wherein the composition is at least 98% pure. For example, the composition is at least 98.1% pure, at least 98.2% pure, is at least 98.3% pure, is at least 98.4% pure, is at least 98.5% pure, is at least 98.6% pure, is at least 98.7% pure, is at least 98.8% pure, is at least 98.9% pure, is at least 99.0% pure, is at least 99.1% pure, is at least 99.2% pure, is at least 99.3% pure, is at least 99.4% pure, is at least 99.5% pure, is at least 99.6% pure, is at least 99.7% pure, is at least 99.8% pure, or is at least 99.9% pure.

In one embodiment, N⁴-(cyclopropylmethyl)-6-[(3R)-3-(methylamino)pyrrolidin-1-yl]pyrimidine-2,4-diamine tartrate dihydrate may be recrystallized to improve the purity of the compound.

The present application also relates to compositions containing N⁴-(cyclopropylmethyl)-6-[(3R)-3-(methylamino)pyrrolidin-1-yl]pyrimidine-2,4-diamine tartrate dihydrate, wherein the compositions further comprises less than 1% of the impurity, 4-N-butyl-6-[(3-(methylamino)pyrrolidin-1-yl]pyrimidine-2,4-diamine (e.g., less than 0.95%, 0.90%, 0.85%, 0.80%, 0.75%, 0.70%, 0.65%, 0.60%, 0.55%, 0.50%, 0.45%, 0.40%, 0.35%, 0.30%, 0.29%, 0.28%, 0.27%, 0.26%, 0.25%, 0.24%, 0.23%, 0.22%, 0.21%, 0.20%, 0.15%, 0.10%, 0.09%, 0.08%, 0.07%, 0.06%, 0.05%, 0.04%, 0.03%, 0.02%, or 0.01% of the impurity). For example, the present application also relates to a composition containing N⁴-(cyclopropylmethyl)-6-[(3R)-3-(methylamino)pyrrolidin-1-yl]pyrimidine-2,4-diamine tartrate dihydrate, wherein the composition may contain less than 1% of the impurity, 4-N-butyl-6-[(3R)-3-methylamino)pyrrolidin-1-yl]pyrimidine-2,4-diamine (e.g., less than 0.95%, 0.90%, 0.85%, 0.80%, 0.75%, 0.70%, 0.65%, 0.60%, 0.55%, 0.50%, 0.45%, 0.40%, 0.35%, 0.30%, 0.29%, 0.28%, 0.27%, 0.26%, 0.25%, 0.24%, 0.23%, 0.22%, 0.21%, 0.20%, 0.15%, 0.10%, 0.09%, 0.08%, 0.07%, 0.06%, 0.05%, 0.04%, 0.03%, 0.02%, or 0.01% of the impurity).

In some embodiments, the compositions containing N⁴-(cyclopropylmethyl)-6-[(3R)-3-(methylamino)pyrrolidin-1-yl]pyrimidine-2,4-diamine tartrate dihydrate and also contain less than 1% of 4-N-butyl-6-[(3-(methylamino)pyrrolidin-1-yl]pyrimidine-2,4-diamine, further comprises less than 0.5% methanol (e.g., less than 0.45%, 0.40%, 0.35%, 0.30%, 0.25%, 0.20%, 0.15%, 0.10%, 0.09%, 0.08%, 0.07%, 0.06%, 0.05%, 0.04%, 0.03%, 0.02%, or 0.01% methanol). By way of non-limiting example, the composition contains between about 0.01% to about 0.05% methanol, about 0.02% to about 0.06% methanol, about 0.03% to about 0.07% methanol, about 0.04% to about 0.08% methanol, about 0.05% to about 0.09% methanol, about 0.01% to about 0.05% methanol, about 0.05% to about 0.1% methanol, about 0.05% to about 0.15% methanol, about 0.05% to about 0.2% methanol, about 0.05% to about 0.25% methanol, about 0.1% to about 0.2% methanol, about 0.1% to about 0.3% methanol, about 0.1% to about 0.4% methanol, about 0.2% to about 0.3% methanol, about 0.2% to about 0.4% methanol, about 0.2% to about 0.5% methanol, about 0.3% to about 0.4% methanol, about 0.3% to about 0.4% methanol, or about 0.4% to about 0.5% methanol.

The present application relates to compositions containing N⁴-(cyclopropylmethyl)-6-[(3R)-3-(methylamino)pyrrolidin-1-yl]pyrimidine-2,4-diamine tartrate dihydrate, wherein the composition is at least 98% pure or where in the composition contains less than 1% of the impurity 4-N-butyl-6-[(3R)-3-methylamino)pyrrolidin-1-yl]pyrimidine-2,4-diamine, wherein the compositions contain a polymorph of N⁴-(cyclopropylmethyl)-6-[(3R)-3-(methylamino)pyrrolidin-1-yl]pyrimidine-2,4-diamine tartrate dihydrate distinguished by Powder X-Ray Diffraction peaks (i.e., PXRD peaks) at about 6.7, 9.2, 22.4, and 24.4 degrees 2-theta.

Such compositions may additionally be distinguished by containing a polymorph with PXRD peaks at about 13.5 and 18.7 degrees 2-theta. Fox example, the compositions containing N⁴-(cyclopropylmethyl)-6-[(3R)-3-(methylamino)pyrrolidin-1-yl]pyrimidine-2,4-diamine tartrate dihydrate are at least 98% pure or contain less than 1% of the impurity 4-N-butyl-6-[(3R)-3-methylamino)pyrrolidin-1-yl]pyrimidine-2,4-diamine and are distinguished by PXRD peaks at about 6.7, 9.2, 13.5, 18.7, 22.4, and/or 24.4 degrees 2-theta.

Likewise, the compositions may additionally be distinguished by containing a polymorph with PXRD peaks at about 20.9, 21.4, 26.8, and 30.0 degrees 2-theta. For example, the compositions containing N⁴-(cyclopropylmethyl)-6-[(3R)-3-(methylamino)pyrrolidin-1-yl]pyrimidine-2,4-diamine tartrate dihydrate are at least 98% pure or contain less than 1% of the impurity 4-N-butyl-6-[(3R)-3-methylamino)pyrrolidin-1-yl]pyrimidine-2,4-diamine and are distinguished by PXRD peaks at about 6.7, 9.2, 13.5, 18.7, 20.9, 21.4, 22.4, 24.4, 26.8, and/or 30.0 degrees 2-theta.

In another embodiment, the compositions may additionally be distinguished by containing a polymorph with PXRD peaks at about 11.4, 15.6, 25.0, and 26.1 degrees 2-theta. For example, the compositions containing N⁴-(cyclopropylmethyl)-6-[(3R)-3-(methylamino)pyrrolidin-1-yl]pyrimidine-2,4-diamine tartrate dihydrate are at least 98% pure or contain less than 1% of the impurity 4-N-butyl-6-[(3R)-3-methylamino)pyrrolidin-1-yl]pyrimidine-2,4-diamine and are distinguished by PXRD peaks at about 6.7, 9.2, 11.4, 13.5, 15.6, 18.7, 20.9, 21.4, 22.4, 24.4, 25.0, 26.1, 26.8, and/or 30.0 degrees 2-theta.

In another embodiment, any of the compositions described herein may additionally be distinguished by containing a polymorph with PXRD peaks at about 17.0, 21.8, and 22.0 degrees 2-theta. For example, the compositions containing N⁴-(cyclopropylmethyl)-6-[(3R)-3-(methylamino)pyrrolidin-1-yl]pyrimidine-2,4-diamine tartrate dihydrate are at least 98% pure or contain less than 1% of the impurity 4-N-butyl-6-[(3R)-3-methylamino)pyrrolidin-1-yl]pyrimidine-2,4-diamine and is distinguished by PXRD peaks at about 6.7, 9.2, 11.4, 13.5, 15.6, 17.0, 18.7, 20.9, 21.4, 21.8, 22.0, 22.4, 24.4, 25.0, 26.1, 26.8, and/or 30.0 degrees 2-theta.

Any of the compositions described herein can be combined with one or more pharmaceutically acceptable carrier(s) or diluent(s) to produce pharmaceutical compositions.

Likewise, an effective amount of any of the compositions and/or pharmaceutical compositions described herein can be made into dosage forms.

By way of non-limiting example, suitable dosage forms can be selected from powder-in-capsule forms, capsules, tablets, liquids (e.g., for inhalation, injection or oral administration), powders (e.g., for inhalation, injection or oral administration), lozenges, chews, multi- and nano-particulates, inhalants, gels, solid solutions, liposomes, nanoparticles, films, ovules, sprays, injectables, liquid formulations, and any combination thereof. For example, the powder-in-capsule may contain the active pharmaceutical ingredient (API) (the powder) in a hydroxypropyl methylcellulose (HPMC) capsule.

In one embodiment, the dosage form is a powder-in-capsule form.

In another embodiment, the dosage form is a tablet form. The tablet form can optionally be film coated.

The present application also relates to methods of treating an H₄ mediated disease or condition by administering an effective amount of any of the compositions or pharmaceutical compositions, or any dosage forms described herein.

Also provided are any of the compositions, pharmaceutical compositions, and/or forms described herein for use in the treatment of an H₄ mediated disease or condition.

The H₄ mediated disease or condition can include, but is not limited to, inflammatory skin diseases, pruritic diseases, respiratory diseases, cardiac diseases, inflammatory diseases of the gastrointestinal tract, cancer, joint diseases, kidney diseases, pain disorders, overactive bladder conditions, vestibular disorders, macular degenerative disorders, inflammatory eye diseases, and/or other diseases involving immune and inflammatory disorders.

The H₄ mediated disease or condition can include, but is not limited to, is an inflammatory skin disease, e.g., atopic dermatitis and/or psoriasis.

In another embodiment, the H₄ mediated disease or condition is a pruritic disease, e.g., urticaria and/or uraemic pruritus.

In a further embodiment, the H₄ mediated disease or condition is a respiratory disease, e.g., asthma, chronic obstructive airway disease, and/or allergic rhinitis.

In one embodiment, the H₄ mediated disease or condition is a cardiac disease, e.g., myocardial ischaemia.

In further embodiments, the H₄ mediated disease or condition is an inflammatory disease of the gastrointestinal tract, e.g., Crohn's disease and/or colitis ulcerosa.

In another embodiment, the H₄ mediated disease or condition is a joint disease, e.g., rheumatoid arthritis and/or psoriatic arthritis.

In another embodiment, the H₄ mediated disease or condition is a kidney disease, e.g., diabetic nephropathy.

In other embodiments, the H₄ mediated disease or condition is a pain disorder, e.g., inflammatory pain and/or neuropathic pain.

In still further embodiments, the H₄ mediated disease or condition is a vestibular disorder, e.g., vertigo and/or tinnitus.

In a different embodiment, the H₄ mediated disease or condition is an inflammatory eye disease, e.g., conjunctivitis and/or uveitis.

In yet another embodiment, the H₄ mediated disease or condition is another disease involving immune and inflammatory disorders, e.g., multiple sclerosis, mastocytosis, and/or inflammatory or systemic lupus erythematosus.

Additional examples of H₄ mediated diseases or conditions include, but are not limited to, atopic dermatitis, bullous disorders, collagenoses, psoriasis, psoriatic lesions, seborrheic dermatitis or contact dermatitis, eczema, urticaria, pruritus, uraemic pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, squamous cell carcinoma and/or melanoma.

In one preferred embodiment, the H₄ mediated disease or condition is psoriasis, atopic dermatitis, and/or other pruritic conditions.

Any of the compositions, pharmaceutical compositions, and/or dosage forms described herein can be administered to the patient via an oral, topical, intravenous, inhalational, otic, intramucosal, intraarterial, intraocular, intraperitoneal, intrathecal, intraventricular, intraurethral, intrasternal, intracranial, intramuscular, and/or subcutaneous route of administration.

Any of the compositions, pharmaceutical compositions, and/or dosage forms described herein can be administered to the patient on a daily (e.g., 1, 2, or 3 times daily), weekly (e.g., 1, 2, 3, 4, or 5 times weekly), or monthly basis (e.g., 1, 2, 3, 4, 5, 6, 7, 8, 9, or 10 times monthly). Determination of the appropriate dosing schedule is within the routine level of skill in the art.

Any of the compositions, pharmaceutical compositions, and/or dosage forms described herein can be administered at a dose of from about 1 mg to about 60 mg. For example, at a dose of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mg.

In various embodiments, the composition, pharmaceutical composition, or dosage form is administered at a dose of from about 1 to about 10 mg, from about 1 to about 15 mg, from about 3 to about 15 mg, from about 5 to about 15 mg, from about 5 to about 20 mg, from about 5 to about 25 mg, from about 5 to about 30 mg, from about 5 to about 35 mg, from about 5 to about 40 mg, from about 5 to about 45 mg, from about 5 to about 50 mg, from about 10 to about 25 mg, from about 10 to about 30 mg, from about 10 to about 35 mg, from about 10 to about 40 mg, from about 10 to about 50 mg, from about 10 to about 60 mg, from about 15 to about 30 mg, from about 15 to about 35 mg, from about 15 to about 40 mg, from about 15 to about 45 mg, from about 20 to about 35 mg, from about 20 to about 40 mg, from about 20 to about 45 mg, from about 20 to about 50 mg, from about 20 to about 55 mg, from about 20 to about 60 mg, from about 25 to about 40 mg, from about 25 to about 50 mg, from about 25 to about 60 mg, from about 30 to about 45 mg, from about 30 to about 55 mg, from about 30 to about 60 mg, from about 35 to about 60 mg, from about 40 to about 50 mg, from about 40 to about 55 mg, from about 40 to about 60 mg, from about 45 to about 60 mg, or from about 50 to about 60 mg.

In one embodiment, pharmaceutical compositions, and/or dosage forms described herein can be administered intravenously, subcutaneously, or intraocularly, at a dose of from about 0.005 to about 100 mg/ml. For example, at a dose of about 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg/ml. In various embodiments, the composition, pharmaceutical composition, or dosage form is administered intravenously, subcutaneously, or intraocularly, at a dose of from 0.05 to about 100 mg/ml, from about 0.01 to about 90 mg/ml, from about 0.005 to about 10 mg/ml, from about 0.05 to about 15 mg/ml, from about 0.5 to about 20 mg/ml, from about 1 to about 10 mg/ml, from about 5 to about 20 mg/ml, from about 10 to about 25 mg/ml, from about 15 to about 25 mg/ml, from about 10 mg/ml to about 30 mg/ml, from about 15 to 35 mg/ml, from about 20 to 40 mg/ml, from about 25 to 45 mg/ml, from about 30 to 50 mg/ml, from about 35 to 55 mg/ml, from about 40 to 60 mg/ml, from about 45 to 65 mg/ml, from about 50 to 70 mg/ml, from about 55 to 75 mg/ml, from about 60 to 80 mg/ml, from about 65 to 85 mg/ml, from about 70 to 90 mg/ml, from about 75 to 95 mg/ml, or from about 80 to 100 mg/ml.

The present application also relates to compositions for treating as well as methods of treating an H₄ mediated disease or condition by administering an effective amount of any of the compositions or pharmaceutical compositions, or any dosage forms described herein, wherein the composition, pharmaceutical composition, or dosage form is administered to the patient with one or more additional therapeutic agents. For example, the one or more additional therapeutic agents can be selected from Histamine H₁ receptor antagonists; Histamine H₃ receptor antagonists; Histamine H₂ receptor antagonists; leukotriene antagonists; phosphodiesterase inhibitors; neurotransmitter re-uptake inhibitors; 5-lipooxygenase (5-LO) inhibitors; 5-lipoxygenase activating protein (FLAP) inhibitors; α₁- and α2-adrenoceptor agonist vasoconstrictor sympathomimetic agents; muscarinic M₃ receptor antagonists or anticholinergic agents; β₂-adrenoceptor agonists; dual acting β₂/M₃ agents; xanthines; non-steroidal anti-inflammatories; ketotifen; COX-1 inhibitors (NSAIDs) and COX-2 selective inhibitors; oral, inhaled intranasal and topical glucocorticosteroids; monoclonal antibodies active against endogenous inflammatory entities; anti-tumor necrosis factor (anti-TNF-α) agents; adhesion molecule inhibitors including VLA-4 antagonists; kinin-B₁- and B₂-receptor antagonists; immunosuppressive agents; inhibitors of matrix metalloproteases (MMPs); tachykinin NK₁, NK₂ and NK₃ receptor antagonists; elastase inhibitors; adenosine A2a receptor agonists; inhibitors of urokinase; compounds that act on dopamine receptors; modulators of the NKκb pathway; agents that can be classed as mucolytics or anti-tussive agents; antibiotics; modulators of cytokine signaling pathways; modulators of the prostaglandin pathways; antagonists of chemokine receptors CXCR1 and CXCR2; antagonists of chemokine receptors CCR3, CCR4 and CCR5; inhibitors of cytosolic and soluble phospholipase A₂ (cPLA₂ and sPLA₂); inhibitors of phosphoinositide-3-kinase; HDAC inhibitors; p38 inhibitors; CXCR2 antagonists; calcineurin inhibitors; anti-interleukin 17 (anti-IL-17) agents; anti-interleukin 4 receptor (anti-IL4R) agents; anti-interleukin 31 (anti-IL-31) agents; CRTH2 antagonists; and combinations thereof.

In one embodiment, the one or more additional therapeutic agents are Histamine H₁ receptor antagonists, including, without limitation, fexofenadine, cetirizine, levocetrizine, loratadine, desloratadine, mepyramine, and diphenhydramine.

In one embodiment, the one or more additional therapeutic agents are leukotriene antagonists, including, without limitation, montelukast, zafirlukast, and pranlukast.

In one embodiment, the one or more additional therapeutic agents are CRTH2 antagonists, including, without limitation, ADC3680, NVP-QAV680, and OC459.

In one embodiment, the one or more additional therapeutic agents are phosphodiesterase inhibitors, including, without limitation, PDE4 phosphodiesterase inhibitors which may be selected from apremilast, roflumilast, and the like.

The present application also relates to compositions for treating as well as methods of treating an H₄ mediated condition by administering an effective amount of a composition containing N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine in combination with one or more additional therapeutic agents, including, without limitation, calcineurin inhibitors, anti-interleukin 17 (anti-IL-17) agents, anti-interleukin 4 receptor (anti-IL-4R) agents, anti-interleukin-31 (anti-IL-31) agents, and combinations thereof to a patient in need thereof. These methods involving combinations with one or more additional therapeutic agents may be administered by the routes and dosages described herein.

The present application also relates to methods of treating an H₄ mediated condition by administering an effective amount of a composition containing N⁴-(cyclopropylmethyl)-6-[(3R)-3-(methylamino)pyrrolidin-1-yl]pyrimidine-2,4-diamine tartrate dihydrate or N⁴-(cyclopropylmethyl)-6-[(3R)-3-(methylamino)pyrrolidin-1-yl]pyrimidine-2,4-diamine to a patient in need thereof, wherein the H₄ mediated condition includes, but is not limited to, atopic dermatitis, urticaria, psoriatic arthritis, vertigo, macular degenerative disorders, mastocytosis, inflammatory lupus erythematosus, systemic lupus erythematosus, bullous disorders, collagenoses, psoriatic lesions, seborrheic dermatitis or contact dermatitis, eczema, pruritus, uraemic pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, neuropathic pain, tinnitus, uveitis, diabetic nephropathy and multiple sclerosis. These methods involving combinations with one or more additional therapeutic agents may be administered by the routes and dosages described herein.

Also provided are tablets containing a therapeutically effective amount of N⁴-(cyclopropylmethyl)-6-[(3R)-3-(methylamino)pyrrolidin-1-yl]pyrimidine-2,4-diamine tartrate dihydrate and one or more pharmaceutically acceptable carriers, diluents or excipients.

In various embodiments, the therapeutically effective amount of N⁴-(cyclopropylmethyl)-6-[(3R)-3-(methylamino)pyrrolidin-1-yl]pyrimidine-2,4-diamine is between 1 and 100 mg. For example, 1 to 60 mg or 30 mg.

In various embodiments, the therapeutically effective amount of N⁴-(cyclopropylmethyl)-6-[(3R)-3-(methylamino)pyrrolidin-1-yl]pyrimidine-2,4-diamine is administered as a corresponding salt, solvate, and/or hydrate. For example, without limitation, N⁴-(cyclopropylmethyl)-6-[(3R)-3-(methylamino)pyrrolidin-1-yl]pyrimidine-2,4-diamine tartrate dihydrate.

In various embodiments, the therapeutically effective amount of N⁴-(cyclopropylmethyl)-6-[(3R)-3-(methylamino)pyrrolidin-1-yl]pyrimidine-2,4-diamine tartrate dihydrate in the tablets is between 1 and 175 mg. For example, 1 to 110 mg, 52 mg, 17.2 mg, 5.2 mg, or 1.7 mg. Any suitable formulation method known in the art can be used to prepare these tablets.

For example, in some embodiments, the tablets are prepared by a dry granulation formulation method.

In some other embodiments, the tablets are prepared by a wet granulation formulation method, a direct compression formulation method, or a moisture activated dry granulation formulation method.

Any of the tablets described herein may additionally contain one or more additional ingredients, such as microcrystalline cellulose (MCC), mannitol, croscarmellose sodium, sodium starch glycolate, dicalcium phosphate anhydrous (DCP), hydroxypropyl cellulose (HPC), povidone, crospovidone, silicon dioxide, magnesium stearate, and/or any other excipients known in the art.

For example, one suitable tablet described herein contains:

(a) about 25.75% by weight of N⁴-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose; and

(c) about 17.85% by weight of dicalcium phosphate anhydrous;

wherein said tablet is prepared by a dry granulation formulation method.

Another suitable tablet described herein contains:

(a) about 25.75% by weight of N⁴-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose; and

(c) about 17.85% by weight of dicalcium phosphate anhydrous.

Such tablets may additionally contain sodium starch glycolate, croscarmellose sodium and/or magnesium stearate.

In some embodiments, any of the excipients used herein may be intra-granular excipients, extra-granular excipients, or a combination thereof. For example, without limitation, microcrystalline cellulose, dicalcium phosphate anhydrous, sodium starch glycolate, croscarmellose sodium, and/or magnesium stearate, may be included as intra-granular excipients, extra-granular excipients, or a combination thereof.

Another suitable tablet described herein contains:

(a) about 25.75% by weight of N⁴-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose;

(c) about 17.85% by weight of dicalcium phosphate anhydrous; and

(d) about 8% by weight of croscarmellose sodium

Another suitable tablet described herein contains:

(a) about 25.75% by weight of N⁴-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose;

(c) about 17.85% by weight of dicalcium phosphate anhydrous;

(d) about 8% by weight of croscarmellose sodium; and

(e) about 1% by weight of a lubricant.

Another suitable tablet described herein contains:

(a) about 25.75% by weight of N⁴-(cyclopropylmethyl)-6-[(3R)-3-(methylamino)pyrrolidin-1-yl]pyrimidine-2,4-diamine tartrate dihydrate;

(b) about 47.4% by weight of microcrystalline cellulose;

(c) about 17.85% by weight of dicalcium phosphate anhydrous;

(d) about 8% by weight of croscarmellose sodium; and

(e) about 1% by weight of magnesium stearate.

Another suitable tablet described herein contains:

(a) about 51.5% by weight of N⁴-(cyclopropylmethyl)-6-[(3R)-3-(methylamino)pyrrolidin-1-yl]pyrimidine-2,4-diamine tartrate dihydrate;

(b) about 19.75% by weight of microcrystalline cellulose; and

(c) about 19.75% by weight of dicalcium phosphate anhydrous;

wherein said tablet is prepared by a wet granulation formulation method.

Another suitable tablet described herein contains:

(a) about 51.5% by weight of N⁴-(cyclopropylmethyl)-6-[(3R)-3-(methylamino)pyrrolidin-1-yl]pyrimidine-2,4-diamine tartrate dihydrate;

(b) about 19.75% by weight of microcrystalline cellulose; and

(c) about 19.75% by weight of dicalcium phosphate anhydrous.

Such tablets may additionally contain one or more of sodium starch glycolate, hydroxypropyl cellulose, and/or magnesium stearate.

In some embodiments, any of the excipients used herein may be intra-granular excipients, extra-granular excipients, or a combination thereof. For example, without limitation, microcrystalline cellulose, dicalcium phosphate anhydrous, sodium starch glycolate, hydroxypropyl cellulose, and/or magnesium stearate, may be included as intra-granular excipients, extra-granular excipients, or a combination thereof.

In other embodiments, the ratio of the microcrystalline cellulose to the dicalcium phosphate anhydrous in the tablet is about 10:1, 9.5:1, 9.0:1, 8.5:1, 8.0:1, 7.5:1, 7.0:1, 6.5:1, 6.0:1, 5.5:1, 5.0:1, 4.5:1, 3.5:1, 3.3:1, 3.0:1, 2.9:1, 2.8:1, 2.7:1, 2.6:1, 2.5:1, 2.4:1, 2.3:1, 2.2:1, 2.1:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.3, 1:3.5, 1:4.0, 1:4.5, 1:5.0, 1:5.5, 1:6.0, 1:6.5, 1:7.0, 1:7.5, 1:8.0, 1:8.5, 1:9.0, 1:9.5, or 1:10.0 by weight. For example, the ratio of the microcrystalline cellulose to the dicalcium phosphate anhydrous may be any of the above, and the microcrystalline cellulose and dicalcium phosphate anhydrous may each be an intra-granular excipient, an extra-granular excipient, or a combination thereof.

Any of the tablets described herein may contain croscarmellose sodium in an amount of about 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10.0%, 10.1%, 10.2%, 10.3%, 10.4%, 10.5%, 10.6%, 10.7%, 10.8%, 10.9%, 11.0%, 11.1%, 11.2%, 11.3%, 11.4%, 11.5%, 11.6%, 11.7%, 11.8%, 11.9%, 12.0%, 12.1%, 12.2%, 12.3%, 12.4%, 12.5%, 12.6%, 12.7%, 12.8%, 12.9%, or 13.0% by weight.

Likewise, any of the tablets may contain sodium starch glycolate in an amount of 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, or 10.0% by weight.

In some embodiments, the tablets may contain hydroxypropyl cellulose (HPC) in an amount of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, or 6.0% by weight.

The tablets may contain a lubricant in amount of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% by weight.

The tablets may contain magnesium stearate in amount of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0% by weight.

In some embodiments, the N⁴-(cyclopropylmethyl)-6-[(3R)-3-(methylamino)pyrrolidin-1-yl]pyrimidine-2,4-diamine tartrate dihydrate and/or any of the pharmaceutically acceptable carriers, diluents, and/or excipients in any of the tablets described herein may be found exclusively inside the granule (i.e., intra-granular) or exclusively outside of the granule (i.e., extra-granular). Alternatively, a combination of intra-granular and extra-granular carriers, diluents, and/or excipients can be used.

The intra-granular: extra-granular ratio of any one of the N⁴-(cyclopropylmethyl)-6-[(3R)-3-(methylamino)pyrrolidin-1-yl]pyrimidine-2,4-diamine tartrate dihydrate, pharmaceutically acceptable carriers, diluents, and/or excipients is about 10:1, 9.5:1, 9.0:1, 8.5:1, 8.0:1, 7.5:1, 7.0:1, 6.5:1, 6.0:1, 5.5:1, 5.0:1, 4.5:1, 3.5:1, 3.3:1, 3.0:1, 2.9:1, 2.8:1, 2.7:1, 2.6:1, 2.5:1, 2.4:1, 2.3:1, 2.2:1, 2.1:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, 1:3.0, 1:3.3, 1:3.5, 1:4.0, 1:4.5, 1:5.0, 1:5.5, 1:6.0, 1:6.5, 1:7.0, 1:7.5, 1:8.0, 1:8.5, 1:9.0, 1:9.5, or 1:10.0 by weight.

Also provided are methods of treating atopic dermatitis in a patient, by administering a tablet comprising a therapeutically effective amount of N⁴-(cyclopropylmethyl)-6-[(3R)-3-(methylamino)pyrrolidin-1-yl]pyrimidine-2,4-diamine tartrate dihydrate and one or more pharmaceutically acceptable carriers, diluents or excipients, to the patient once daily.

By way of non-limiting example, the therapeutically effective amount of N⁴-(cyclopropylmethyl)-6-[(3R)-3-(methylamino)pyrrolidin-1-yl]pyrimidine-2,4-diamine is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg.

By way of non-limiting example, the tablets described herein may contain 1, 1.5, 1.7, 2, 2.5, 3, 3.5, 4, 4.5, 5, 5.2, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.2, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60, 60.5, 61, 61.5, 62, 62.5, 63, 63.5, 64, 64.5, 65, 65.5, 66, 66.5, 67, 67.5, 68, 68.5, 69, 69.5, 70, 70.5, 71, 71.5, 72, 72.5, 73, 73.5, 74, 74.5, 75, 75.5, 76, 76.5, 77, 77.5, 78, 78.5, 69, 79.5, 80, 80.5, 81, 81.5, 82, 82.5, 83, 83.5, 84, 84.5, 85, 85.5, 86, 86.5, 87, 87.5, 88, 88.5, 89, 89.5, 90, 90.5, 91, 91.5, 92, 92.5, 93, 93.5, 94, 94.5, 95, 95.5, 96, 96.5, 97, 97.5, 98, 98.5, 99, 99.5, 100, 100.5, 101, 101.5, 102, 102.5, 103, 103.5, 104, 104.5, 105, 105.5, 106, 106.5, 107, 107.5, 108, 108.5, 109, 109.5, 110, 110.5, 111, 111.5, 112, 112.5, 113, 113.5, 114, 114.5, 115, 115.5, 116, 116.5, 117, 117.5, 118, 118.5, 119, 119.5, 120, 120.5, 121, 121.5, 122, 122.5, 123, 123.5, 124, 124.5, 125, 125.5, 126, 126.5, 127, 127.5, 128, 128.5, 129, 129.5, 130, 130.5, 131, 131.5, 132, 132.5, 133, 133.5, 134, 134.5, 135, 135.5, 136, 136.5, 137, 137.5, 138, 138.5, 139, 139.5, 140, 140.5, 141, 141.5, 142, 142.5, 143, 143.5, 144, 144.5, 145, 145.5, 146, 146.5, 147, 147.5, 148, 148.5, 149, 149.5, 150, 150.5, 151, 151.5, 152, 152.5, 153, 153.5, 154, 154.5, 155, 155.5, 156, 156.5, 157, 157.5, 158, 158.5, 159, 159.5, 160, 160.5, 161, 161.5, 162, 162.5, 163, 163.5, 164, 164.5, 165, 165.5, 166, 166.5, 167, 167.5, 168, 168.5, 169, 169.5, 170, 170.5, 171, 171.5, 172, 172.5, 173, 173.5, 174, 174.5, or 175 mg of N⁴-(cyclopropylmethyl)-6-[(3R)-3-(methylamino)pyrrolidin-1-yl]pyrimidine-2,4-diamine tartrate dihydrate.

Moreover, any of the compositions, pharmaceutical compositions, and/or dosage forms described herein can be used to inhibit, interfere, disrupt, etc. the ability of histamine to bind the H₄ receptor. Likewise, any of the compositions, pharmaceutical compositions, and/or dosage forms can also be used to inhibit, interfere, disrupt, etc., the binding of an agonist of the H₄ receptor (e.g., 4-Methylhistamine, VUF-8430 (2-[(Aminoiminomethyl)amino]ethyl carbamimidothioic acid ester), or OUP-16) or of an antagonist of the H₄ receptor (e.g., Thioperamide, JNJ 7777120, or VUF-6002 (1-[(5-Chloro-1H-benzimidazol-2-yl)carbonyl]-4-methylpiperazine)).

### Isomers

Any of the compounds described herein, or a pharmaceutically acceptable salt, or solvate, e.g., hydrate or dihydrate, or prodrug thereof, may exist as geometric isomers (i.e., cis-trans isomers), optical isomers or stereoisomers, such as diastereomers, as well as tautomers. Accordingly, it should be understood that the definition of any of the compositions, pharmaceutical compositions, or dosage forms described herein includes each and every individual isomer corresponding to the structural formula of the compound contained therein, or a pharmaceutically acceptable salt, or solvate thereof, e.g., hydrate or dihydrate, including cis-trans isomers, stereoisomers and tautomers, as well as racemic mixtures of these. Further, any of the compositions, pharmaceutical compositions, or dosage forms described herein are also intended to encompass all R- and S-isomers of a chemical structure in any ratio, e.g., with enrichment (i.e. enantiomeric excess or diastereomeric excess) of one of the possible isomers and corresponding smaller ratios of other isomers.

Diastereoisomers, i.e., non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of the application, or a pharmaceutically acceptable salt, or solvate, or prodrug thereof, with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. Optically active compounds of the application, or a pharmaceutically acceptable salt, or solvate, e.g., hydrate or dihydrate, or prodrug thereof, can likewise be obtained by utilizing optically active starting materials and/or by utilizing a chiral catalyst. These isomers may be in the form of a free acid, a free base, an ester or a salt. Examples of chiral separation techniques are given in Chiral Separation Techniques, A Practical Approach, 2^{nd} ed. by G. Subramanian, Wiley-VCH, 2001, which is incorporated herein by reference it its entirety.

### Isotopic Variations

Elemental symbols and element names are used herein also include isotopes of the named elements. In particular one, some, or all hydrogens may be deuterium. Radioactive isotopes may be used, for instance to facilitate tracing the fate of the compounds or their metabolic products after administration.

### Methods for Preparing Compounds of the Application

The present application relates to methods of producing N⁴-(cyclopropylmethyl)-6-[(3R)-3-(methylamino)pyrrolidin-1-yl]pyrimidine-2,4-diamine tartrate dihydrate, which includes the following steps:

a) crystallizing N⁴-(cyclopropylmethyl)-6-[(3R)-3-(methylamino)pyrrolidin-1-yl]pyrimidine 2,4-diamine tartrate from an aqueous solution of N⁴-(cyclopropylmethyl)-6-[(3R)-3-(methylamino)pyrrolidin-1-yl]pyrimidine-2,4-diamine tartrate;

b) isolating the crystallized material; and

c) drying the isolated material under wet inert gas flow until such time that the water content of the isolated material is between 6 and 10% and any organic solvent present comprises <0.5% of the isolated material;

wherein the isolated material comprises N⁴-(cyclopropylmethyl)-6-[(3R)-3-(methylamino)pyrrolidin-1-yl]pyrimidine-2,4-diamine tartrate dihydrate.

In these methods, the isolated material can include a polymorph of N⁴-(cyclopropylmethyl)-6-[(3R)-3-(methylamino)pyrrolidin-1-yl]pyrimidine-2,4-diamine tartrate dihydrate. For example, the isolated polymorph of N⁴-(cyclopropylmethyl)-6-[(3R)-3-(methylamino)pyrrolidin-1-yl]pyrimidine-2,4-diamine tartrate dihydrate can be distinguished by PXRD peaks at about 6.7, 9.2, 22.4, and 24.4 degrees 2-theta.

Such polymorphs may be additionally distinguished by PXRD peaks at about 13.5 and 18.7 degrees 2-theta. For example, the isolated polymorph of N⁴-(cyclopropylmethyl)-6-[(3R)-3-(methylamino)pyrrolidin-1-yl]pyrimidine-2,4-diamine tartrate dihydrate can be distinguished by PXRD peaks at about 6.7, 9.2, 13.5, 18.7, 22.4, and/or 24.4 degrees 2-theta.

Likewise, such polymorphs may be additionally distinguished by PXRD peaks at about 20.9, 21.4, 26.8, and 30.0 degrees 2-theta. For example, the isolated polymorph of N⁴-(cyclopropylmethyl)-6-[(3R)-3-(methylamino)pyrrolidin-1-yl]pyrimidine-2,4-diamine tartrate dihydrate can be distinguished by PXRD peaks at about 6.7, 9.2, 13.5, 18.7, 20.0, 21.4, 22.4, 24.4, 26.8, and/or 30.0 degrees 2-theta.

Also provided are methods for producing N⁴-(cyclopropylmethyl)-6-[(3R)-3-(methylamino)pyrrolidin-1-yl]pyrimidine-2,4-diamine tartrate dihydrate, including the following steps:
a) adding an amount of N⁴-(cyclopropylmethyl)-6-[(3R)-3-(methylamino)pyrrolidin-1-yl]pyrimidine-2,4-diamine (2R,3R)-tartrate to a volume of purified water to produce a first solution and warming to a temperature above 50° C. (e.g., from about 55° C. to about 65° C.);
b) charging the first solution with an organic solvent to produce a second solution;
c) cooling the second solution to 40-60° C. (e.g., to about 50° C. over a period of about 20 to about 60 minutes, about 30 to 90 minutes, about 45 to 180 minutes, about 60 to 240 minutes, or to about 40° C. over a period of about 20 to about 60 minutes, about 30 to 90 minutes, about 45 to 180 minutes, about 60 to 240 minutes, or to about 30° C. over a period of about 20 to about 60 minutes, about 30 to 90 minutes, about 45 to 180 minutes, about 60 to 240 minutes) to produce a slurry;
d) progressively cooling the slurry to 20-35° C.;
e) isolating the slurry;
f) washing the isolated material; and
g) drying the isolated material under wet inert gas flow until such time that the water content of the isolated material is between 6 and 10% and any organic solvent present comprises <0.5% of the isolated material;
wherein the isolated material comprises a polymorph of N⁴-(cyclopropylmethyl)-6-[(3R)-3-(methylamino)pyrrolidin-1-yl]pyrimidine-2,4-diamine tartrate dihydrate.

The amount of organic solvent in the isolated N⁴-(cyclopropylmethyl)-6-[(3R)-3-(methylamino)pyrrolidin-1-yl]pyrimidine-2,4-diamine tartrate dihydrate, or a polymorph thereof, can be determined using nuclear magnetic resonance (NMR) or gas chromatography (GC).

For any of the methods for producing N⁴-(cyclopropylmethyl)-6-[(3R)-3-(methylamino)pyrrolidin-1-yl]pyrimidine-2,4-diamine tartrate dihydrate described herein, the aqueous solution of N⁴-(cyclopropylmethyl)-6-[(3R)-3-(methylamino)pyrrolidin-1-yl]pyrimidine-2,4-diamine tartrate is treated with an organic solvent. For example, the organic solvent may be an alcohol, e.g., methanol, ethanol, n-propanol, or iso-propanol. In a preferred embodiment, the organic solvent is methanol.

For any of the methods described herein for producing N⁴-(cyclopropylmethyl)-6-[(3R)-3-(methylamino)pyrrolidin-1-yl]pyrimidine-2,4-diamine tartrate dihydrate described herein, the isolated material is dried under wet inert gas flow wherein the inert gas, e.g., argon, nitrogen, or helium. In one preferred embodiment, the inert gas is nitrogen.

For any of the methods described herein for producing N⁴-(cyclopropylmethyl)-6-[(3R)-3-(methylamino)pyrrolidin-1-yl]pyrimidine-2,4-diamine tartrate dihydrate described herein, the relative water humidity in the drying chamber, i.e., the location where the isolated material is dried under wet inert gas flow, is more than about 40% RH. For example, the relative humidity in the drying chamber may be about 45% to about 99% RH, about 50% to about 99% RH, about 55% to about 99% RH, about 60% to about 99% RH, about 65% to about 99% RH, about 66% to about 99% RH, about 67% to about 99% RH, about 68% to about 99% RH, about 69% to about 99% RH, about 70% to about 99% RH, about 71% to about 99% RH, about 72% to about 99% RH, about 73% to about 99% RH, about 74% to about 99% RH, about 75% to about 99% RH, about 80% to about 99% RH, about 85% to about 99% RH, about 90% to about 99% RH, about 75% to about 99% RH, about 80% to about 99% RH. In other embodiments, the relative humidity in the drying chamber may be about 40% RH and about 60% RH, about 45% RH and about 65% RH, about 50% RH and about 70% RH, about 55% RH and about 75% RH, about 60% RH and about 80% RH, about 65% RH and about 85% RH, about 70% RH and about 90% RH, about 75% RH and about 95% RH, or about 88% RH and 99% RH.

For methods described herein for producing N⁴-(cyclopropylmethyl)-6-[(3R)-3-(methylamino)pyrrolidin-1-yl]pyrimidine-2,4-diamine tartrate dihydrate, the N⁴-(cyclopropylmethyl)-6-[(3R)-3-(methylamino)pyrrolidin-1-yl]pyrimidine-2,4-diamine tartrate is crystallized by progressively cooling the aqueous solution of N⁴-(cyclopropylmethyl)-6-[(3R)-3-(methylamino)pyrrolidin-1-yl]pyrimidine-2,4-diamine tartrate. Likewise, in another embodiment, a polymorph of N⁴-(cyclopropylmethyl)-6-[(3R)-3-(methylamino)pyrrolidin-1-yl]pyrimidine-2,4-diamine tartrate is crystallized by progressively cooling the aqueous solution of a polymorph of N⁴-(cyclopropylmethyl)-6-[(3R)-3-(methylamino)pyrrolidin-1-yl]pyrimidine-2,4-diamine tartrate.

### Methods of Treatment

The present application relates to compositions for treatment of as well as methods of treating an H₄ mediated disease or condition. Any of the compositions, pharmaceutical compositions, dosage forms, and/or any combinations thereof may be used to treat such H₄ mediated disease or conditions. Likewise, any of the compositions, pharmaceutical compositions, dosage forms, and/or any combinations thereof are for treatment of an H₄ mediated disease or condition.

The H₄ mediated disease or condition includes, without limitation, the following diseases and conditions: inflammatory skin diseases, pruritic diseases and conditions, respiratory diseases, cardiac diseases, inflammatory diseases of the gastrointestinal tract, cancer, joint diseases, kidney diseases, pain disorders, overactive bladder conditions, vestibular disorders, macular degenerative disorders, inflammatory eye diseases, and other diseases involving immune and inflammatory disorders.

By way of non-limiting example, the inflammatory skin disease is atopic dermatitis or psoriasis; the pruritic disease is urticaria or uraemic pruritus; the respiratory disease is asthma, chronic obstructive airway disease, or allergic rhinitis; the cardiac disease is myocardial ischaemia; the inflammatory disease of the gastrointestinal tract is Crohn's disease or colitis ulcerosa; the joint disease is rheumatoid arthritis or psoriatic arthritis; the kidney disease is diabetic nephropathy, the pain disorder is inflammatory pain or neuropathic pain; the vestibular disorder is vertigo or tinnitus; the inflammatory eye disease is conjunctivitis or uveitis; the other disease involving immune and inflammatory disorders is multiple sclerosis, mastocytosis, or inflammatory or systemic lupus erythematosus.

Additionally, any of the compositions or pharmaceutical compositions or any combinations thereof may be used to treat an H₄ mediated disease or condition selected from bullous disorders, collagenoses, psoriasis, psoriatic lesions, seborrheic dermatitis or contact dermatitis, eczema, urticaria, uraemic pruritus, pruritus, rosacea, prurigo nodularis, hypertrophic scarring, keloid scar formation, scleroderma, Folliculitis keloidalis nuchae, Kawasaki Disease, Sjögren-Larsson Syndrome, Grover's disease, a first degree burn, a second degree burn, a third degree burn, a fourth degree burn, cutaneous mucinosis, solar keratosis, squamous cell carcinoma and melanoma.

### Pharmaceutically and Veterinarily Acceptable Salts

The present application relates to compositions containing a pharmaceutically and/or veterinarily acceptable salt of N⁴-(cyclopropylmethyl)-6-[(3R)-3-(methylamino)pyrrolidin-1-yl]pyrimidine-2,4-diamine.

Pharmaceutically and/or veterinarily acceptable salts refer to salts of any of the compounds described herein, which are considered to be acceptable for clinical and/or veterinary use. Typical pharmaceutically acceptable salts include those salts prepared by reaction of the compounds with a mineral or organic acid or an organic or inorganic base. Such salts are known as acid addition salts and base addition salts, respectively. These salts may be prepared by any methods known to the skilled person. Pharmaceutically acceptable salts are, e.g., those described and discussed in Remington's Pharmaceutical Sciences, 17. Ed. Alfonso R. Gennaro (Ed.), Mack Publishing Company, Easton, Pa., U.S.A., 1985 and more recent editions thereof, as well as in the Encyclopedia of Pharmaceutical Technology, all of which are incorporated herein by reference.

For example, pharmaceutically and/or veterinarily acceptable salt may include, without limitation, acid addition salts, including both mono- and di-salts, formed with inorganic acids e.g., hydrochloric, hydrobromic, sulfuric, nitric, hydroiodic, metaphosphoric, or phosphoric acid; and organic acids e.g., succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, oxalic, methanesulfonic or naphthalenesulfonic acid. Further, the pharmaceutically and/or veterinarily acceptable salts, including mono- and di-salts, described herein, further include their corresponding solvates, e.g., hydrates and dihydrates.

In one embodiment, the composition contains a gentisate salt, a salicylate salt, a di-hydrochloride salt, and/or an ethane disulfonate salt of N⁴-(cyclopropylmethyl)-6-[(3R)-3-(methylamino)pyrrolidin-1-yl]pyrimidine-2,4-diamine.

Pharmaceutical compositions containing pharmaceutically and/or veterinarily acceptable salts of N⁴-(cyclopropylmethyl)-6-[(3R)-3-(methylamino)pyrrolidin-1-yl]pyrimidine-2,4-diamine may additionally contain one or more pharmaceutically and/or acceptable carrier(s) and/or diluent(s).

Likewise, an effective amount of any of the compositions described herein that contain a pharmaceutically and/or veterinarily acceptable salt of N⁴-(cyclopropylmethyl)-6-[(3R)-3-(methylamino)pyrrolidin-1-yl]pyrimidine-2,4-diamine, e.g., the gentisate salt, salicylate salt, di-hydrochloride salt, or ethane disulfonate salt, or any pharmaceutical compositions thereof can be made into a dosage form. By way of non-limiting example, suitable dosage forms can be selected from powder-in-capsule forms, capsules, tablets, liquids (e.g., for inhalation, injection or oral administration), powders (e.g., for inhalation, injection or oral administration), lozenges, chews, multi- and nano-particulates, gels, solid solutions, liposomes, nanoparticles, films, ovules, sprays, injectables, liquid formulations, and any combination thereof. For example, the powder in capsule may contain the active pharmaceutical ingredient (API) (the powder) in a hydroxypropyl methylcellulose (HPMC) capsule.

In one embodiment, the dosage form is a powder-in-capsule form.

In another embodiment, the dosage form is a tablet form. The tablet form can optionally be film coated.

### Pharmaceutical Compositions and Dosage Forms

Also provided herein are pharmaceutical compositions containing, as an active ingredient, at least one composition or pharmaceutical composition of the application, or a pharmaceutically acceptable salt, or solvate, e.g., hydrate or dihydrate, or prodrug thereof, and optionally one or more pharmaceutically acceptable excipients, diluents and/or carriers. The compositions or pharmaceutical compositions of the application, or a pharmaceutically acceptable salt, or solvate, e.g., hydrate or dihydrate, or prodrug thereof, may be administered alone or in combination with pharmaceutically acceptable carriers, diluents or excipients, in either single or multiple doses. Suitable pharmaceutically acceptable carriers, diluents and excipients include, but are not limited to, inert solid diluents or fillers, sterile aqueous solutions and various organic solvents.

Any of the compositions or pharmaceutical compositions described herein may be formulated with pharmaceutically acceptable carriers or diluents as well as any other known adjuvants and excipients in accordance with conventional techniques such as those disclosed in Remington: The Science and Practice of Pharmacy, 21^{st} Edition, 2000, Lippincott Williams & Wilkins, which is incorporated herein in its entirety.

The pharmaceutical compositions formed by combining compositions or pharmaceutical compositions described herein, or a pharmaceutically acceptable salt, or solvate, e.g., hydrate or dihydrate, or prodrug thereof, as defined herein with pharmaceutically acceptable carriers, diluents or excipients can be readily administered in a variety of dosage forms such as tablets, powders (e.g., for inhalation, injection or oral administration), lozenges, syrups, suppositories, injectable solutions and the like. In powders, e.g., for inhalation, injection or oral administration, the carrier is a finely divided solid such as microcrystalline cellulose or starch which is in a mixture with the finely divided active component. In tablets, the active component is mixed with the carrier having the necessary binding properties in suitable proportions and compacted in the shape and size desired.

The compositions or pharmaceutical compositions may be specifically prepared for administration by any suitable route such as the oral and parenteral (including inhalational, otic, intramucosal, subcutaneous, intramuscular, intrathecal, intravenous and intradermal) route. It will be appreciated that the preferred route will depend on the general condition and age of the subject to be treated, the nature of the condition to be treated and the active ingredient chosen.

Compositions or pharmaceutical compositions for oral administration include solid dosage forms such as capsules, tablets, dragees, pills, lozenges, powders, e.g., for inhalation, injection or oral administration, and granules. Where appropriate, they can be prepared with coatings such as enteric or aesthetic coatings or they can be prepared so as to provide controlled release of the active ingredient such as sustained or prolonged release according to methods well known in the art. In some embodiments, compositions or pharmaceutical compositions for oral administration include solid dosage forms such as capsules, tablets, dragees, pills, lozenges, powders, and granules. Tablets may be optionally prepared with an aqueous film such as Opadry® II, including, without limitation, Opadry® II (brown) or Opadry® II (white).

For oral administration in the form of a tablet or capsule, compositions or pharmaceutical compositions, or a pharmaceutically acceptable salt, or solvate, e.g., hydrate or dihydrate, or prodrug thereof, as defined herein may suitably be combined with one or more oral, non-toxic, pharmaceutically acceptable carrier, diluent, and/or excipient. Suitable carriers, diluents and excipients include, without limitation, fillers, binders, lubricants, disintegrating agents, glidants (e.g., silicon dioxide), flavoring agents and colorants. Suitable binders include, e.g., microcrystalline cellulose (e.g., Avicel PH200 LM, PH112, PH101, PH102, PH103, PH113, PH105, PH200, DG), mannitol, dicalcium phosphate, dicalcium phosphate anhydrous, povidone, lactose, glucose, starch, gelatin, acacia gum, tragacanth gum, sodium alginate, carboxymethylcellulose, polyethylene glycol, waxes or the like. Lubricants include, e.g., glyceryl dibehenate (Compritol®), hydrogenated vegetable oil (Lubritab®), sodium oleate, sodium stearate, magnesium stearate, silicon dioxide, sodium benzoate, sodium acetate, sodium chloride or the like. Disintegrating agents include, e.g., starch, methyl cellulose, agar, bentonite, xanthan gum, sodium starch glycolate, crospovidone, croscarmellose sodium or the like. Additional excipients for capsules include macrogols or lipids.

For the preparation of solid compositions or pharmaceutical compositions such as tablets, the active compositions of the application, or a pharmaceutically acceptable salt, or solvate, e.g., hydrate or dihydrate, or prodrug thereof, is mixed with one or more excipients, such as the ones described above, and other pharmaceutical diluents such as water to make a solid pre-formulation composition containing a homogenous mixture of compositions or pharmaceutical compositions, or a pharmaceutically acceptable salt, or solvate, e.g., hydrate or dihydrate, or prodrug thereof. The term “homogenous” is understood to mean that the compositions or pharmaceutical compositions, or a pharmaceutically acceptable salt, or solvate, e.g., hydrate or dihydrate, or prodrug thereof, is dispersed evenly throughout the composition so that the composition may readily be subdivided into equally effective unit dosage forms such as tablets or capsules.

Liquid compositions for either oral or parenteral administration of the compositions or pharmaceutical compositions, or a pharmaceutically acceptable salt, or solvate, e.g., hydrate or dihydrate, or prodrug thereof, include, e.g., aqueous solutions, syrups, elixirs, aqueous or oil suspensions and emulsion with edible oils such as cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose or polyvinylpyrrolidone.

Compositions or pharmaceutical compositions for parenteral administration include sterile aqueous and non-aqueous injectable solutions, dispersions, suspensions or emulsions as well as sterile powders, e.g., for inhalation, injection or oral administration, to be reconstituted in sterile injectable solutions or dispersions prior to use. For parenteral administration, solutions containing compositions or pharmaceutical compositions, or a pharmaceutically acceptable salt, or solvate, e.g., hydrate or dihydrate, or prodrug thereof, in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The oily solutions are suitable for intra-articular, intra-muscular and subcutaneous injection purposes.

The preparation of all these solutions under sterile conditions is readily accomplished by standard pharmaceutical techniques well known to those skilled in the art.

Depot injectable compositions or pharmaceutical compositions are also contemplated as being within the scope of the present application.

In addition to the aforementioned ingredients, the compositions or pharmaceutical compositions, or a pharmaceutically acceptable salt, or solvate, e.g., hydrate or dihydrate, or prodrug thereof, may optionally include one or more additional ingredients such as diluents, buffers, flavoring agents, colorant, surface active agents, thickeners, preservatives, e.g., methyl hydroxybenzoate (including anti-oxidants), emulsifying agents and the like. However, in some embodiments, the ultra-pure composition(s) described herein is included in white HPMC capsules without any additional formulation components. Moreover, these dosage forms can optionally be film coated.

A suitable dosage of any of the compositions described herein, or pharmaceutical compositions thereof, will depend on the age and condition of the patient, the severity of the disease to be treated, and other factors well known to the practicing physician. The compositions or pharmaceutical compositions may be administered to the patient via a number of routes, including without limitation, via an oral, topical, inhalational, otic, intramucosal, intravenous, intraarterial, intraocular, intraperitoneal, intrathecal, intraventricular, intraurethral, intrasternal, intracranial, intramuscular, or subcutaneous route of administration. Further, different dosing schedules, e.g., bi-daily, daily or with intervals, such as weekly intervals will depend on the aforementioned factors. The compositions or pharmaceutical composition may be administered as a bolus (i.e., the entire daily dose is administered at once) or in divided doses two or more times a day. Variations based on the aforementioned dosage ranges may be made by a physician of ordinary skill taking into account known considerations such as weight, age, and condition of the person being treated, the severity of the affliction, and the particular route of administration.

The compositions of the application, or a pharmaceutically acceptable salt, or solvate, e.g., hydrate or dihydrate, or prodrug thereof, may also be prepared in a pharmaceutical composition containing one or more further active substances alone, or in combination with pharmaceutically acceptable carriers, diluents, or excipients in either single or multiple doses. The suitable pharmaceutically acceptable carriers, diluents and excipients are as described herein, and the one or more further active substances may be any active substances, or preferably an active substance as described herein.

### Formulations

The present application also relates to the development of scalable, robust, processable solid formulations containing ZPL-389 and processes for preparing such formulations. For example, the formulation may be in any of the forms (tablet, pill, capsule, etc.) described herein. For example, the formulation of ZPL-389 may be in the form of a tablet containing about 1 to 100 mg of ZPL-389, about 1 to 90 mg of ZPL-389, about 1 to 80 mg of ZPL-389, about 1 to 70 mg of ZPL-389, about 1 to 60 mg of ZPL-389, about 1 to 50 mg of ZPL-389, about 1 to 40 mg of ZPL-389, about 1 to 30 mg of ZPL-389, about 1 to 20 mg of ZPL-389, or about 1 to 10 mg of ZPL-389. In certain embodiments, the formulation may contain 3 mg, 10 mg, or 30 mg of ZPL-389. To insure content uniformity (CU), the target tablet weights are maintained between 100 mg and 500 mg (i.e., 100, 125, 150, 175, 200, 225, 250, 275, 300, 325, 350, 375, 400, 425, 450, 475, or 500 mg) in order to achieve drug loading of more than 1%.

For example, the robust and processable formulation containing ZPL-389 may be prepared by dry granulation (e.g., roller compaction or slugging and milling), wet granulation, direct compression, and/or moisture activated dry granulation. Any suitable formulation methods known in the art may be used.

Dry granulation involves the formation of granules without using a liquid solution. This requires compacting and densifying the active pharmaceutical ingredient (API)/pharmaceutically acceptable carriers, diluents, and/or excipient powders. After the powders are properly compacted, they may be passed through a mill and final blend prior to tablet compression.

Wet granulation involves the formation of granules by the addition of a granulation liquid onto a powder bed, which may be under the influence of an impeller, one or more screws, and/or air. After formation of the granules, the granulation liquid is removed by drying.

Direct compression involves the blending of an API with one or more pharmaceutically acceptable carriers, diluents, and/or excipients, followed by compression.

Moisture activated dry granulation involves two stages: (1) agglomeration and (2) moisture distribution. During agglomeration, a major portion of the API is blended with one or more pharmaceutically acceptable carriers, diluents, and/or excipients. In the next stage, a small amount of water is sprayed as small droplets onto the blend while blending, forming moist agglomerates. The remaining portion of the API and one or more pharmaceutically acceptable carriers, diluents, and/or excipients are added to and blended with the moist agglomerates.

A summary of various formulations of ZPL-389 and their characteristics is detailed in Table 1.

TABLE 1
Summary of Formulations of ZPL-389

| | Tablet | | | | | |
|---|---|---|---|---|---|---|
| Formulation | strength | Force | Carr's | | | |
| Type | & weight | hardness | index | Disintegration | Manufacturability | Dissolution |
| Wet | 30 mg in | Steep | 17% | 8-13 min | Good | Poor |
| Granulation | 100 mg | | | | | |
| Dry | 30 mg in | Steep | 31% | 3-8 min | Good | Good |
| Granulation | 200 mg | | | | | |
| Direct | 30 mg in | NA | 41% | NA | Very Poor | NA |
| compression | 200 mg | | | | | |
| Direct | 10 mg in | Steep | 23% | 1-2 min (ring | OK | NA |
| compression | 100 mg | | | formation) | | |
| Moisture | 30 mg in | Very | 27% | 1-3 min (ring | Non-standard | NA |
| activated | 200 mg | steep | | formation) | process. Poor | |
| dry | | | | | robustness. | |
| granulation | | | | | | |

Additional improvements and/or alterations to any of the formulations described herein may be found by modifying the ratios of ZPL-389 to any of the pharmaceutically acceptable carriers, diluents, and/or excipients; modifying any of the pharmaceutically acceptable carriers, diluents, and/or excipients; and/or modifying the ratios of the intra-granular and/or extra-granular pharmaceutically acceptable carriers, diluents, and/or excipients. Determination of suitable improvements and/or alterations to the formulations is within the routine level of skill in the art.

Formulations containing ZPL-389 prepared by dry granulation (roller compaction or slugging and milling), wet granulation, direct compression, or moisture activated dry granulation, may be combined with any suitable oral, non-toxic, pharmaceutically acceptable carrier known in the art, including, but not limited to, ethanol, glycerol, water or the like. Furthermore, any suitable binders, lubricants, disintegrating agents, glidants, flavoring agents and/or colorants known in the art may be added to the mixture, as appropriate. Suitable binders include, but are not limited to microcrystalline cellulose (e.g., Avicel PH200 LM, PH112, PH101, PH102, PH103, PH113, PH105, PH200, DG), mannitol, dicalcium phosphate, dicalcium phosphate anhydrous or povidone. Lubricants include, e.g., magnesium stearate, calcium stearate, zinc stearate, fatty acids (e.g., stearic acid, myristic acid, palmitic acid), glyceryl dibehenate (Compritol®), hydrogenated vegetable oil (Lubritab®), sodium oleate, sodium stearate, silicon dioxide, sodium benzoate, sodium acetate, sodium chloride. Disintegrating agents include, e.g., sodium starch glycolate, crospovidone, and/or croscarmellose sodium. Suitable glidants include, e.g., silicon dioxide.

The tablet formulations disclosed herein may be evaluated for future development and scale-up in a number of ways. For example, the physical characteristics of the tablets may be determined and evaluated, for instance, overall strength (amount of API), overall weight, hardness, friability, homogeneity, manufacturability, and the like.

Additionally, the tablets can also be evaluated by the dissolution characteristics as described in Example 15 (infra). Improvements to the dissolution profiles of any the formulations described herein can be achieved by modifying the disintegrant, the disintegrant amount in the formulation, and/or the ratio of intra-granular and extra-granular excipient levels. For example, the formulations may contain about 1%, 2%, 3%, 4%, 5%, 6%, 7%, 8%, 9% or 10% by weight of the disintegrant. Those skilled in the art will recognize that dissolution characteristics can be an important differentiator between tablets prepared by different formulation methods.

The tablets can also be evaluated by their disintegration characteristics. For example, the tablets described in Examples 11-16 (infra) disintegrate from the center of the tablet first, and completely disintegrate on the order of seconds or minutes, typically about 1-10 minutes.

### Combination Treatment

Any of the compositions and/or pharmaceutical compositions described herein may be administered to the patients via any of the dosage forms and routes described herein, and may also be administered to the patient with one or more additional therapeutic agents are selected from the group consisting of Histamine H₁ receptor antagonists; Histamine H₃ receptor antagonists; Histamine H₂ receptor antagonists; leukotriene antagonists; phosphodiesterase inhibitors; neurotransmitter re-uptake inhibitors; 5-lipooxygenase (5-LO) inhibitors; 5-lipoxygenase activating protein (FLAP) inhibitors; α₁- and α₂-adrenoceptor agonist vasoconstrictor sympathomimetic agents; muscarinic M₃ receptor antagonists or anticholinergic agents; β₂-adrenoceptor agonists; dual acting β₂/M₃ agents; xanthines; non-steroidal anti-inflammatories; ketotifen; COX-1 inhibitors (NSAIDs) and COX-2 selective inhibitors; oral, inhaled intranasal and topical glucocorticosteroids; monoclonal antibodies active against endogenous inflammatory entities; anti-tumor necrosis factor (anti-TNF-α) agents; adhesion molecule inhibitors including VLA-4 antagonists; kinin-B₁- and B₂-receptor antagonists; immunosuppressive agents; inhibitors of matrix metalloproteases (MMPs); tachykinin NK₁, NK₂ and NK₃ receptor antagonists; elastase inhibitors; adenosine A2a receptor agonists; inhibitors of urokinase; compounds that act on dopamine receptors; modulators of the NFκb pathway; agents that can be classed as mucolytics or anti-tussive agents; antibiotics; modulators of cytokine signaling pathways; modulators of the prostaglandin pathways; antagonists of chemokine receptors CXCR1 and CXCR2; antagonists of chemokine receptors CCR3, CCR4 and CCR5; inhibitors of cytosolic and soluble phospholipase A₂ (cPLA₂ and sPLA₂); inhibitors of phosphoinositide-3-kinase; HDAC inhibitors; p38 inhibitors; CXCR2 antagonists; calcineurin inhibitors; anti-interleukin 17 (anti-IL-17) agents; anti-interleukin 4 receptor (anti-IL4R) agents; anti-interleukin 31 (anti-IL-31) agents; CRTH2 antagonists; and combinations thereof.

In certain embodiments, the Histamine H₁ receptor antagonists include, without limitation, fexofenadine, cetirizine, levocetrizine, loratadine, desloratadine, mepyramine, and diphenhydramine.

In certain embodiments, the leukotriene antagonists include, without limitation, montelukast, zafirlukast, and pranlukast.

In certain embodiments, CRTH2 antagonists include, without limitation, ADC3680, NVP-QAV680, and OC459.

In certain embodiments, PDE4 phosphodiesterase inhibitors include, without limitation, apremilast and roflumilast.

Compositions or pharmaceutical compositions described herein, or a pharmaceutically acceptable salt, or solvate, e.g., hydrate or dihydrate, or prodrug thereof, as defined herein, may also be used to advantage in combination with other known therapeutic processes, e.g., the administration of hormones or tumor cell damaging approaches, especially ionizing radiation.

### General Procedures

N⁴-(cyclopropylmethyl)-6-[(3R)-3-(methylamino)pyrrolidin-1-yl]pyrimidine-2,4-diamine may be prepared according to the manner described in U.S. Pat. No. 7,943,628, which is incorporated herein in its entirety by reference.

For example, N⁴-(cyclopropylmethyl)-6-[(3R)-3-(methylamino)pyrrolidin-1-yl]pyrimidine-2,4-diamine may be prepared from reacting tert-butyl [(3R)-1-(2-amino-6-chloropyrimidin-4-yl)pyrrolidin-3-yl]methyl-carbamate and cyclopropylmethylamine with di-isopropyl-ethylamine in N-methyl-2-pyrrolidone. Subsequent removal of the tert-butyloxycarbonyl protecting group under acidic conditions (HCl in dioxane and methanol) provides N⁴-(cyclopropylmethyl)-6-[(3R)-3-(methylamino)pyrrolidin-1-yl]pyrimidine-2,4-diamine. Tert-butyl [(3R)-1-(2-amino-6-chloropyrimidin-4-yl)pyrrolidin-3-yl]methyl-carbamate may be prepared from reacting 2-amino-4,6-dichloropyrimidine and tert-butyl (R)-methyl(pyrrolidin-3-yl)carbamate with triethylamine in isopropanol. (See U.S. Pat. No. 7,943,628).

Potentially useful salts of N⁴-(cyclopropylmethyl)-6-[(3R)-3-(methylamino)pyrrolidin-1-yl]pyrimidine-2,4-diamine were identified through a salt screening procedure. The initial salt screen was done with 32 counter ions in a 1 to 1 stoichiometric ratio to the API. The vast majority of the experiments resulted in an amorphous powder or oily substance. However, some experiments resulted in crystalline solids. These solids were evaluated on their physical stability by short-term accelerated stress incubation. As a result of this stress test, some samples reverted to an amorphous powder or were hygroscopic to the level of becoming deliquescent. Stable, non-hygroscopic salts were subjected to a more detailed physico-chemical analysis before the final candidates were selected.

In addition to the one to one stoichiometry, an attempt was also made to prepare two to one stoichiometric salts. From the 14 counter ions tested in these experiments, only hydrochloric acid led to a stable di-hydrochloride salt of N⁴-(cyclopropylmethyl)-6-[(3R)-3-(methylamino)pyrrolidin-1-yl]pyrimidine-2,4-diamine.

Stable crystalline salts of N⁴-(cyclopropylmethyl)-6-[(3R)-3-(methylamino)pyrrolidin-1-yl]pyrimidine-2,4-diamine with Gentisic acid, Salicylic acid, Ethane disulfonic acid and Hydrochloric acid are described herein.

Salts of N⁴-(cyclopropylmethyl)-6-[(3R)-3-(methylamino)pyrrolidin-1-yl]pyrimidine-2,4-diamine can generally be prepared by dissolving N⁴-(cyclopropylmethyl)-6-[(3R)-3-(methylamino)pyrrolidin-1-yl]pyrimidine-2,4-diamine in an appropriate solvent followed by addition of the corresponding organic or inorganic acid or diacid, e.g., gentisic acid, salicylic acid, hydrochloric acid, ethane disulfonic acid. Alternatively, salts of N⁴-(cyclopropylmethyl)-6-[(3R)-3-(methylamino)pyrrolidin-1-yl]pyrimidine-2,4-diamine can be prepared by adding a solution of N⁴-(cyclopropylmethyl)-6-[(3R)-3-(methylamino)pyrrolidin-1-yl]pyrimidine-2,4-diamine to the corresponding organic or inorganic acid or diacid, e.g., gentisic acid, salicylic acid, hydrochloric acid, ethane disulfonic acid. Based on the nature of the acid or diacid to be added to N⁴-(cyclopropylmethyl)-6-[(3R)-3-(methylamino)pyrrolidin-1-yl]pyrimidine-2,4-diamine, warming or cooling of the reaction solutions may be necessary. The salts can be isolated by removing solvent, purified by conventional means such filtration or recrystallization. Further drying under heat and/or vacuum may be necessary.

X-ray powder diffraction (XRPD) may be used to identify and/or characterize any of the crystalline solids described herein. See Examples 1-3, infra.

The invention having been described, the following examples are offered by way of illustration and not limitation.

### EXAMPLES

### Example 1. Analytical Methods

High-Throughput X-Ray Powder Diffraction:

XRPD patterns were obtained using the Crystallics T2 high-throughput XRPD set-up. The plates were mounted on a Bruker General Area Detector Diffraction System (GADDS) equipped with a VANTEC-500 gas area detector corrected for intensity and geometric variations. The calibration of the measurement accuracy (peaks position) was performed using NIST SRM1976 standard (Corundum).

Data collection was carried out at room temperature using monochromatic CuK_{α} radiation in the 2θ region between 1.5° and 41.5°, which is the most distinctive part of the XRPD pattern. The diffraction pattern of each well was collected in two 20 ranges (1.5°≤2θ≤21.5° for the first frame, and 19.5°≤2θ≤41.5° for the second) with an exposure time of 90s for each frame. No background subtraction or curve smoothing was applied to the XRPD patterns.

The carrier material used during XRPD analysis was transparent to X-rays and contributed only slightly to the background.

### Thermal Analysis-DSC:

Melting properties were obtained from DSC thermograms, recorded with a heat flux DSC822e instrument (Mettler-Toledo GmbH, Switzerland). The DSC822e was calibrated for temperature and enthalpy with a small piece of indium (m.p.=156.6° C.; δHf=28.45 J/g). Samples (circa 2 mg) were sealed in standard 40 μL aluminum pans, pin-holed and heated in the DSC from 25° C. to 300° C., at a heating rate of 10° C./min. Dry N₂ gas, at a flow rate of 50 mL/min was used to purge the DSC equipment during measurement.

### Thermal Analysis-DSC/TGMS:

Mass loss due to solvent or water loss from the crystals was determined by TGA/SDTA. Monitoring the sample weight, during heating in a TGA/SDTA851e instrument (Mettler-Toledo GmbH, Switzerland), resulted in a weight vs. temperature curve. The TGA/SDTA851e was calibrated for temperature with indium and aluminum. Samples (circa 2 mg) were weighed into 100 μL aluminum crucibles and sealed. The seals were pin-holed and the crucibles heated in the TGA from 25 to 300° C. at a heating rate of 10° C./min. Dry N₂ gas was used for purging.

The gases evolved from the TGA samples were analyzed by an Omnistar GSD 301 T2 mass spectrometer (Pfeiffer Vacuum GmbH, Germany). This MS is a quadrupole mass spectrometer, which analyses masses in the range of 0-200 amu.

HPLC Analytical Method:

### HPLC: Agilent 1200

Detector 1: DAD set at 284 nm, Detector 2: HP1100 LC/MSD in Positive Scan mode
HPLC Conditions: Autosampler temp: 15° C.

### Column: Waters Sunfire C18 (100×4.6 mm; 3.5 μm).

Column temp: 35° C. Flowcel: 10 mm path
Gradient: Mobile phase A: 10 mM Ammonium acetate
Mobile phase B: Acetonitrile
Flow: 1.0 ml/min
HPLC mobile phase gradient.

| Time | Eluent | Eluent |
|---|---|---|
| 0 | 90% | 10% |
| 1 | 90% | 10% |
| 6 | 10% | 90% |
| 9 | 10% | 90% |
| 10 | 90% | 10% |

Concentration: ca. 0.8 mg/mL
Solvent: 10 mM Ammonium acetate: Acetonitrile (50:50 v/v) Injection volume: 5 μl

The compound integrity is expressed as a peak-area percentage, calculated from the area of each peak in the chromatogram, except the ‘injection peak’, and the total peak-area, as follows:

$$\text{?} = peak - area \% ( peak - area ) / ( total - area ) * 100 \%$$

$$\text{?} \text{indicates text missing or illegible when filed}$$

The peak-area percentage of the compound of interest is employed as an indication of the purity of the component in the sample.

Dynamic Vapor Sorption:

Moisture sorption isotherms were collected on a DVS-1 system from Surface Measurement Systems (London, UK). Typical sample size is between 5 and 10 mg of solid material. The relative humidity was cycled between 40% to 0% (desorption), up to 95% (sorption), back to 0% RH (desorption) and up to 95% RH (sorption) in steps of 10% at a constant temperature of 25° C. with an initial stabilizing step at 40% RH for 6 hours.

### Example 2. Determination of ZPL-3893787-18 by HPLC

This analytical method describes the HPLC procedure applied for the identification and determination of both ZPL-3893787-18 and related substances in ZPL-3893787-18 drug substance, present in ZPL-3893787-18 capsules (30 mg active moiety).

Apparatus:
Clean Grade A glassware
Suitable liquid chromatograph equipped with mobile phase degasser, gradient pump, UV detector capable of operating at 230 nm, a sample injection system of 10 μL capacity and data acquisition system or integrator.
HPLC Column—Gemini 5 μm C18, 150×4.6 mm (or equivalent).

Reagents:

Acetonitrile, HPLC Grade

Ammonium Hydroxide 28-30%, Reagent grade

Perchloric Acid, 70% ACS Grade or equivalent

Purified Water for HPLC use (e.g. Milli-Q or equivalent)

Preparation of Solutions:

Sample solvent (0.1% Ammonium Hydroxide (aq)/Acetonitrile (90/10))

5.0 mL of ammonium hydroxide is added to 5000 mL of purified water and mixed well. 900 mL of 0.1% ammonium hydroxide (aq) is added to 100 mL of Acetonitrile and mixed well.

Mobile Phase A—0.1% Ammonium Hydroxide (aq)

5.0 mL of ammonium hydroxide is added to 5000 mL of purified water and mixed well, followed by degassing by sonication for 10 minutes.

Mobile Phase B—0.1% Perchloric Acid in Acetonitrile

2.0 mL of perchloric acid is added to 2000 mL of acetonitrile, mixed well, and degassed by sonication for 10 minutes.

Preparation of Standard Solutions

ZPL-3893787-18 Assay Working Standard Solution (240 μg/mL)

Approximately 24 mg±0.5 mg of ZPL-3893787-18 reference standard is added into a 100 mL volumetric flask. 80 mL of sample solvent is added and sonicated for 5 minutes to dissolve. Dilution to volume with sample solvent, followed by thorough mixing provides the “Working Standard Solution.”

ZPL-3893787-18 Related Substances Standard Solution (2.4 μg/mL)

1.0 mL of the “Assay Reference Standard Preparation” is diluted to 100.0 mL with sample solvent and mixed well. This is the “Working Related Substances Standard Solution.”

Concentrated LOQ Solution (Related Substances Only) (60 μs/mL)

5.0 mL of the “Working Standard Solution” is diluted to 20.0 mL with sample solvent and mixed well. This is the “Concentrated LOQ Solution”.

Working LOQ Solution (Related Substances Only) (0.6 μg/mL)

1.0 mL of the “Concentrated LOQ Solution” is diluted to 100.0 mL with sample solvent and mixed well. This is the “Working LOQ Solution”.

Preparation of Sample Solutions

API Samples—Related Substances

120 mg±1.0 mg of ZPL-3893787-18 API is added to a 100 mL volumetric flask. A 80 mL of sample solvent was added and sonicated for 5 minutes to dissolve. Dilution to volume with sample solvent followed by thorough mixing provides the “Related Substances Sample”.

API Samples—Assay Samples

20 mL of “Related Substances Sample” is added to a 100 mL volumetric flask and diluted with 80 mL of sample solvent and sonicated for 5 minutes to dissolve. Dilution to volume with sample solvent followed by thorough mixing provides the “Assay Sample”.

Capsule Samples—Related Substances

The contents of 10 capsules were emptied and carefully mixed. 120 mg of capsule contents was weighed into a 100 mL volumetric flask. 80 mL of sample solvent was added and sonicated for 5 minutes to dissolve. Dilution to volume with sample solvent followed by thorough mixing provides the “Capsule Related Substances Sample”.

Capsule Samples—Assay Samples

20 mL of “Capsule Related Substances sample” is added to a 100 mL volumetric flask. and diluted with 80 mL of sample solvent and sonicated for 5 minutes to dissolve. Dilution to volume with sample solvent followed by thorough mixing provides the “Capsule Assay Sample”.

Chromatographic Details

| Column | Gemini 5 μm C18, 150 × 4.6 mm, | | |
|---|---|---|---|
| | or equivalent (pH range 2-11) | | |
| Column | Ambient | | |
| Temperature | | | |
| Flow Rate | 1.0 mL/minute | | |
| Detector | UV at λ 230 nm | | |
| Injection volume | 10 μL | | |
| Run time | 44 minutes | | |
| | Gradient | | |
| | Time | % Mobile Phase A | Mobile Phase B |
| Mobile phase | 0 | 90 | 10 |
| composition | 37.0 | 30 | 70 |
| | 39.0 | 30 | 70 |
| | 39.1 | 90 | 10 |
| | 44.0 | 90 | 10 |

Chromatographic Sequence

A sequence of Standard Solutions, Resolution Solution, LOQ Solution and Sample Solutions is injected. An example sequence is presented below:

| Injection | | Number of |
|---|---|---|
| Number* | Standard/Sample | Injections |
| 1 | Sample Solvent | 1 |
| 2 | Assay Working Standard A | 1 |
| 3 | Assay Working Standard B | 1 |
| 4 | Assay Working Standard A | 1 |
| 5 | Assay Working Standard A | 1 |
| 6 | Assay Working Standard A | 1 |
| 7 | Assay Working Standard A | 1 |
| 8 | Assay Working Standard A | 1 |
| 9 | Sample Solvent | 1 |
| 10 | Related Substances Working Standard A | 1 |
| 11 | Related Substances Working Standard B | 1 |
| 12 | Sample Solvent | 1 |
| 13 | LOQ Solution | 1 |
| 14 | Assay Working Standard A | 1 |
| 15 | Related Substances Working Standard A | 1 |
| 16 | Sample 1 | 1 |
| 17 | Sample 2 | 1 |
| 18 | Sample 3 | 1 |
| 19 | Sample 4 | 1 |
| 20 | Sample 5 | 1 |
| 21 | Sample 6 | 1 |
| 22 | Sample 7 | 1 |
| 23 | Sample 8 | 1 |
| 24 | Working Standard A | 1 |
| 25 | Related Substances Working Standard A | 1 |
| 26 | SLOWFLOW | NA |

*The injection number does not relate to the vial location on the instrument.

Multiple injections from a single vial can be taken, for example Working Standard A. Not more than 10 sample injections are to be performed between standard injections. To aid analysis, the sequence may be run as a series of smaller sequences, for example injection number 1 to 14 and injection number 14 to 25. When this approach is applied, the system suitability criteria is applied to the first sequence. Consecutive sequences are evaluated against the bracket standards as long as the injector precision is maintained. Consecutive sequences may only be run if the instrument conditions have not been altered, for example the introduction of fresh mobile phase will require a repeat of the system suitability sequence.

Results Interpretation:

Peak areas are determined for all of the peaks of interest. The approximate retention time of the drug substance is 13 minutes.

| Peak Name | Relative Retention Time | Assignment |
|---|---|---|
| Unknown | 0.31 | Degradation Impurity |
| PF-04188744 | 0.88 | Process Impurity |
| ZPL-3893787-18 | 1.00 | API |
| PF-04360799 | 1.17 | Process Impurity |
| PF-04626829 | 1.90 | Process Impurity |
| PF-01012688 | 2.10 | StartingMaterial |

System Suitability

The following system suitability criteria is applied:

| System Suitability Test | Acceptance Criteria |
|---|---|
| Injector Precision of Working Standard A (n = 6) | ≤2.0 RSD |
| Injector Precision of Working Standard A | ≤2.0 RSD |
| Standard Recovery/Agreement | 98.0 to 102.0% |
| Selectivity | No interfering peaks at |
| | the retention times of |
| | ZPL-3893787-18 |
| LOQ signal to noise ratio for the | ≥10:1 |
| ZPL-3893787-18 peak | |

Identity

The identity of ZPL-3893787-18 is confirmed by comparison of the retention time of the ZPL-3893787-18 peak observed in the sample with that observed in the Working Standard Solution. Identity is confirmed when the retention time of the ZPL-3893787-18 peak in the sample (RT1) corresponds to that in the Working Standard Solution (RT2) within 0.98 to 1.02 when RT1/RT2.

Assay and Related Substances Calculation

The content of ZPL-3893787-18 in the capsules is calculated as follows:

$$ZPL \text{-} 3893787 \text{-} 18 content ( \% w / w ) = \frac{Asam}{Astd} \times \frac{Wstd}{100} \times \frac{p}{100} \times \frac{100}{Wsam} \times 100$$

Where:
Astd=Average of the ZPL-3893787-18 peak area in Assay Standard Solution
Wstd=Weight of ZPL-3893787-18 Reference Standard (mg)
Wsam=Weight of sample (mg)
Asam=Area of ZPL-3893787-18 in the sample solution
P=Purity of ZPL-3893787-18 Reference Standard (%)

The content of each known and unknown Related Substances was calculated as follows:

$$ZPL \text{-} 3893787 \text{-} 18 content ( \% w / w ) = \frac{Asam}{Astd} \times \frac{Wstd}{100} \times \frac{p}{100} \times DF \times \frac{100}{Wsam} \times 100$$

Where:
Astd=Average of the ZPL-3893787-18 peak area in Working Related Substances Standard Solution
Wstd=Weight of ZPL-3893787-18 Reference Standard (mg) Wsam=Weight of sample (mg)
Asam=Area of ZPL-3893787-18 in the sample solution
P=Purity of ZPL-3893787-18 Reference Standard (%)
DF=Dilution factor of related substances Standard (0.01)

Reporting Criteria

The ZPL-3893787-18 is reported as the mean of the individual results of the replicate sample solution preparations. All known and unknown related substances greater than or equal to 0.05% with their respective relative retention times are also reported.

### Example 3. Salt Screen

The tartrate salt of N⁴-(cyclopropylmethyl)-6-[(3R)-3-(methylamino)pyrrolidin-1-yl]pyrimidine-2,4-diamine is a channel hydrate which may contain variable amounts of water which may affect its manufacturing negatively.

The screen commenced with the preparation of the free base from the tartrate salt of N⁴-(cyclopropylmethyl)-6-[(3R)-3-(methylamino)pyrrolidin-1-yl]pyrimidine-2,4-diamine. Then salt formation with 32 counter-ions and two solvents (1-butanol and dichloromethane). The counter-ions assessed in the salt screen are listed in Table 2.

TABLE 2

| # | Counter-ion |
|---|---|
| 1 | Hydrobromic acid |
| 2 | Hydrochloric acid |
| 3 | Sulfuric acid |
| 4 | Ethane-1,2-disulfonic acid |
| 5 | p-Toluenesulfonic acid |
| 6 | Methanesulfonic acid |
| 7 | Naphtalene-2-sulfonic acid |
| 8 | Benzenesulfonic acid |
| 9 | Oxalic acid |
| 10 | L-Aspartic acid |
| 11 | Maleic acid |
| 12 | Phosphoric acid |
| 13 | Ethanesulfonic acid |
| 14 | Glutamic acid |
| 15 | Gentisic acid |
| 16 | Salicylic acid |
| 17 | (+)-L-Tartaric acid |
| 18 | Fumaric acid |
| 19 | Citric acid |
| 20 | D-Glucuronic acid |
| 21 | DL-Mandelic acid |
| 22 | (−)-L-Malic acid |
| 23 | Hippuric acid |
| 24 | Gluconic acid |
| 25 | Lactic acid |
| 26 | L-Ascorbic acid |
| 27 | Benzoic acid |
| 28 | Succinic acid |
| 29 | 4-acetamido-Benzoic acid |
| 30 | Glutaric acid |
| 31 | Acetic acid |
| 32 | Stearic acid |

The initial results of the salt formation experiments are summarized in Table 3. Approximately 175 mg of N⁴-(cyclopropylmethyl)-6-[(3R)-3-(methylamino)pyrrolidin-1-yl]pyrimidine-2,4-diamine in 1-Butanol and Dichloromethane were combined with a slight molar excess of either solid or liquid counter ion. Samples were incubated at RT. After 5 days the solids were collected, dried under vacuum and analyzed by HT-XRPD (XRPD 1).

Subsequently, the solids were exposed to accelerated aging conditions (40° C., 75% RH) for two days and re-analyzed by HT-XRPD (XRPD 2). Crystalline salts (polymorphs) are indicated with the abbreviated counter ion code and a number (1, 2, etc.). The number zero (0) indicates the crystalline counter ion itself.

TABLE 3

| | 1-Butanol | | Dichloromethane | |
|---|---|---|---|---|
| Counter-ion | XRPD1 | XRPD2 | XRPD1 | XRPD2 |
| Hydrobromic acid | Amorphous | Amorphous | Amorphous | Amorphous |
| Hydrochloric acid | HCl1 | Amorphous | Amorphous | Amorphous |
| Sulfuric acid | Sul1 + Sul2 | Amorphous | Sul1 | Amorphous |
| Ethane-1,2-disulfonic acid | Eds2 | Eds1 + Eds2 | Eds1 | Eds1 |
| p-Toluenesulforic acid | Amorphous | Amorphous | Amorphous | Amorphous |
| Methanesulfonic acid | Amorphous | Amorphous | Amorphous | Amorphous |
| Naphtalene-2-sulfonic acid | Amorphous | Nap1 + Nap0 | Amorphous | Amorphous |
| Benzenesulfonic acid | Amorphous | Amorphous | Amorphous | Amorphous |
| Oxalic acid | Oxa2 | Oxa3 | Oxa1 | Oxa3 |
| L-Aspartic acid | Asp1 + Asp0 | Asp0 | Asp1 + Asp0 | Amorphous |
| Maleic acid | Mae1 | Amorphous | Amorphous | Amorphous |
| Phosphoric acid | Pho2 | Pho2 plus | Pho1 | Pho3 |
| Ethanesulfonic acid | Amorphous | Amorphous | Amorphous | Amorphous |
| Glutamic acid | Gltm0 | Gltm0 | Gltm1 + Gltm0 | Amorphous |
| Gentisic acid | Gen2 | Gen2 | Gen1 | Gen2 |
| Salicylic acid | Sal1 plus | Sal1 plus | Amorphous | Amorphous |
| (+)-L-Tartaric acid | Tar2 | Amorphous | Tar2 | Amorphous |
| Fumaric acid | Fum2 | Amorphous | Fum0 + Fum1 | Fum0 + Fum3 |
| Citric acid | Cit0 | Amorphous | Cit1 + Cit0 | Amorphous |
| D-Glucuronic acid | Amorphous + Gluc0 | Amorphous | Amorphous + Gluc0 | Amorphous |
| DL-Mandelic acid | Mad1 | Amorphous | Amorphous | Amorphous |
| (−)-L-Malic acid | Amorphous | Amorphous | Mal1 | Amorphous |
| Hippuric acid | Hip1 | Amorphous | Hip1 | Amorphous |
| Gluconic acid | Amorphous | Amorphous | Amorphous | Amorphous |
| Lactic acid | Lac2 | Amorphous | Lac1 | Amorphous |
| L-Ascobic acid | Asc0 | Amorphous | Asc0 | Amorphous |
| Benzoic acid | Ben1 + Ben2 | Ben1 + Ben2 plus | Ben1 + Ben2 | Ben1 + Ben2 plus |
| Succinic acid | Suc1 + Suc2 | Suc1 | Suc1 | Suc1 |
| 4-acetamido-Benzoic acid | ABen1 + ABen0 | ABen1 + ABen0 | ABen1 + ABen0 | ABen1 + ABen0 |
| Glutaric acid | Amorphous | Glut1 | Amorphous | Glut1 |
| Acetic acid | Amorphous | Amorphous | Ace1 | Amorphous |
| Stearic acid | Ste2 + Ste0 | Ste0 + Ste1 | Ste1 | Ste0 + Ste1 |

The majority of the samples obtained in the primary salt screen were amorphous or oily in appearance. In order to improve on the crystallinity, all solids obtained in the salt screen were recrystallized in ethyl acetate, tetrahydrofuran, toluene, acetonitrile and anisole respectively. The recrystallized solids were analyzed by HT-XRPD before and after exposure to accelerated aging conditions (40° C., 75% RH). The results of the recrystallization experiments show that despite using a diversity of solvents for the recrystallization procedure, many of the salts remained amorphous. However, in some cases recrystallization resulted in a crystalline solid, e.g., oxalic acid, phosphoric acid, gentisic acid, salicylic acid, fumaric acid, benzoic acid, ethane-1,2-disulfonic acid, and 4-acetamido benzoic. Some of these crystalline solids were not stable upon exposure to accelerated aging conditions (40° C., 75% RH). Only those solids that remained crystalline were subjected to a more detailed analysis. These are summarized in Table 4.

TABLE 4

| | HPLC | SDTA | | | TGA | |
|---|---|---|---|---|---|---|
| | Purity | Melt | Decomposition | Weight less | Range | |
| Counter-ion | % | (° C.) | (° C.) | (° C.) | (° C.) | Solvent ID |
| Ethane-1,2-disulfonic acid (Eds1) | 98 | — | 229 | 1 | 25-190 | Water |
| Ethane-1,2-disulfonic acid (Eds2) | 99 | — | 218 | 7.1 | 25-160 | Toluene |
| Oxalic acid | 98 | — | 165 | 4.8 | 25-145 | Toluene |
| Phosphoric acid | 99 | 144 | 220 | 3.2 | 25-180 | Ethylacetate |
| Gentisic acid (Gen1) | 96 | — | 224 | 2.1 | 25-140 | Water |
| Gentisic acid (Gen2) | 97 | 134 | 230 | 1.5 | 25-150 | Water |
| Salicylic acid | 99 | 173 | 192 | 1.5 | 25-160 | Water |
| Fumaric acid | 96 | 168 | 175 | 5.7 | 25-160 | Ethylacetate |
| Benzoic acid | 87 | — | 156 | 2.5 | 25-160 | Tetrahydrofuran |
| Succinic acid | 97 | 105 | 160 | 3.1 | 25-110 | Tetrahydrofuran |
| 4-Acetamindo-benzoic acid | 93 | 138 | 243 | 3.3 | 25-180 | Tetrahydrofuran + Water |

Additionally, similar screening experiments were conducted in to form a crystalline di-salt of N⁴-(cyclopropylmethyl)-6-[(3R)-3-(methylamino)pyrrolidin-1-yl]pyrimidine-2,4-diamine. The acids examined were: hydrobromic acid, hydrochloric acid, sulfuric acid, ethane-1,2-disulfonic acid, p-toluenesulfonic acid, methanesulfonic acid, naphthalene-2-sulfonic acid, benzene sulfonic acid, oxalic acid, L-aspartic acid, glutamic acid, (+)-L-tartaric acid, fumaric acid, and citric acid. From these acids, only the di-HCl salt of N⁴-(cyclopropylmethyl)-6-[(3R)-3-(methylamino)pyrrolidin-1-yl]pyrimidine-2,4-diamine provided crystalline material.

### Example 4. Preparation of Ultra-Pure N⁴-(cyclopropylmethyl)-6-[(3R)-3-(methylamino)pyrrolidin-1-yl]pyrimidine-2,4-diamine tartrate dihydrate

N⁴-(cyclopropylmethyl)-6-[(3R)-3-(methylamino)pyrrolidin-1-yl]pyrimidine-2,4-diamine (4.21 g, 16.04 mmol) was dissolved in methanol (14 mL) and water (14 mL) and heated to 60° C. A solution of L-tartaric acid (2.4 g, 16.04 mmol) in methanol (14 mL) was added and rinsed with additional methanol (14 mL). The clear solution was continuously heated at 60° C. for about 10 minutes before solid started to form. The suspension was cooled to 50° C. for 1.5 hours, then cooled to 40° C. over 30 minutes and held at 40° C. for 2 hours. The suspension was then cooled to 30° C. over 30 minutes and held at 30° C. overnight. The solid was collected by filtration, washed with methanol (20 mL), and dried under vacuum at room temperature with a beaker of water inside the oven for 2 days to provide an ultra-pure form of N⁴-(cyclopropylmethyl)-6-[(3R)-3-(methylamino)pyrrolidin-1-yl]pyrimidine-2,4-diamine tartrate dihydrate white solid (6.617 grams, 86.9%). There was no further weight loss after the first day of drying. This ultra-pure material may be recrystallized (See Example 5). N⁴-(cyclopropylmethyl)-6-[(3R)-3-(methylamino)pyrrolidin-1-yl]pyrimidine-2,4-diamine tartrate dihydrate prepared by this procedure was determined to have purity>98% by LCMS. In contrast, when measured by LCMS, the purity of N⁴-(cyclopropylmethyl)-6-[(3R)-3-(methylamino)pyrrolidin-1-yl]pyrimidine-2,4-diamine tartrate dihydrate made by the methods described in U.S. Pat. No. 7,943,628 had purity (measured by LCMS) of 95.4% and 96.1% assay.

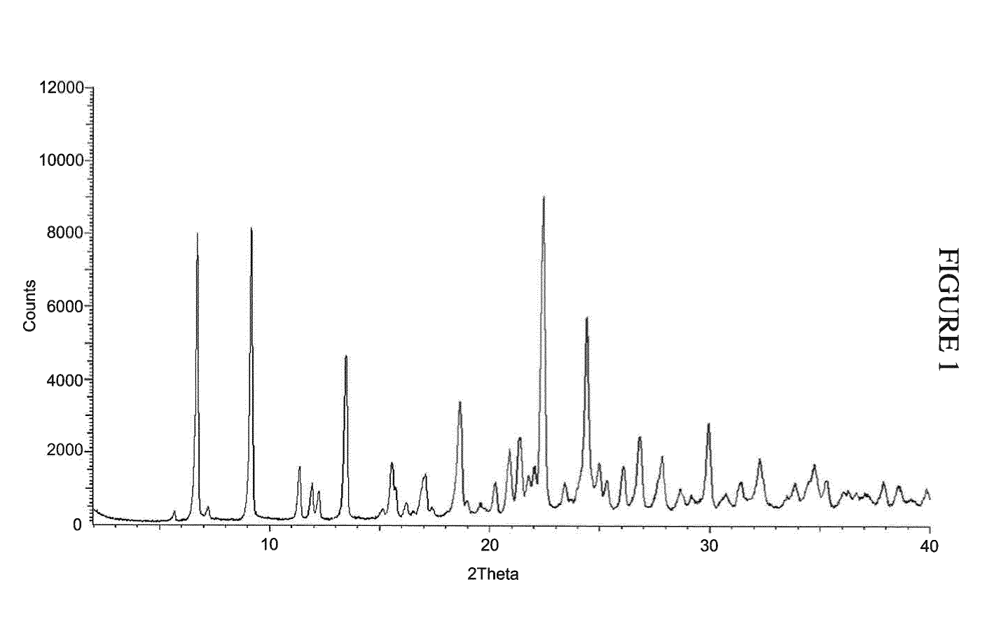

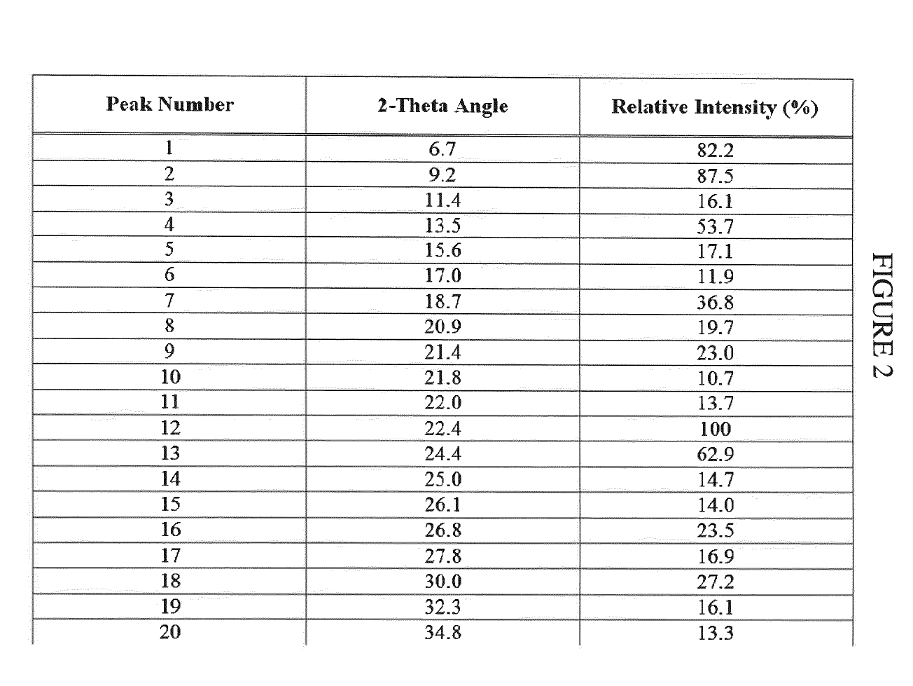

Ultra-pure N⁴-(cyclopropylmethyl)-6-[(3R)-3-(methylamino)pyrrolidin-1-yl]pyrimidine-2,4-diamine tartrate dihydrate, as prepared above provides a polymorph form A, characterized by the PXRD pattern and PXRD peak listings in FIG. 1 and FIG. 2, respectively.

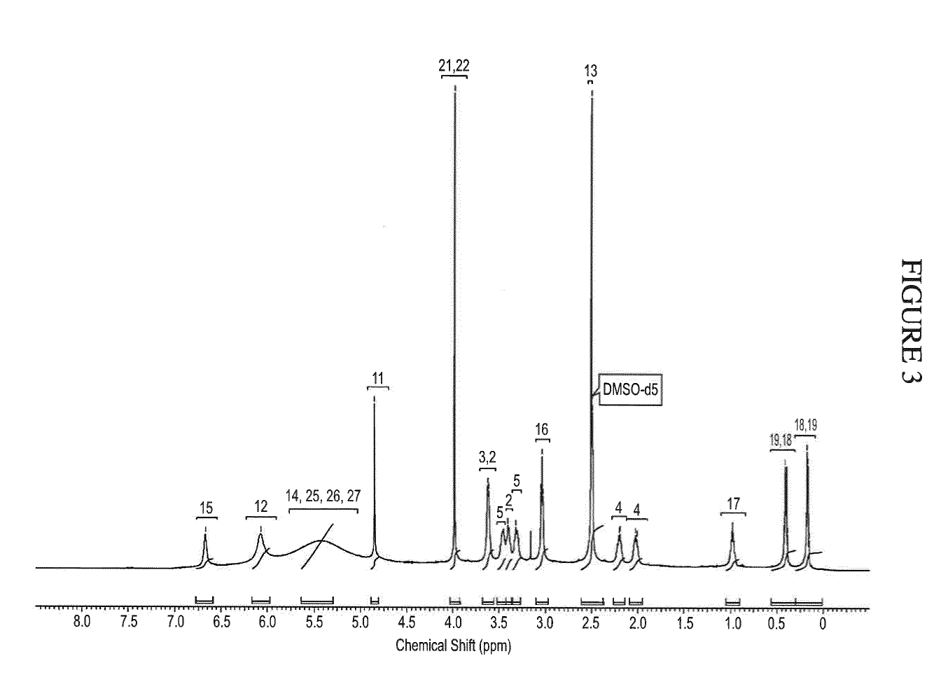

¹H NMR: (FIG. 3) ¹H NMR of ultra-pure N⁴-(cyclopropylmethyl)-6-[(3R)-3-(methylamino)pyrrolidin-1-yl]pyrimidine-2,4-diamine tartrate dihydrate (Form A) in DMSO-d₆.

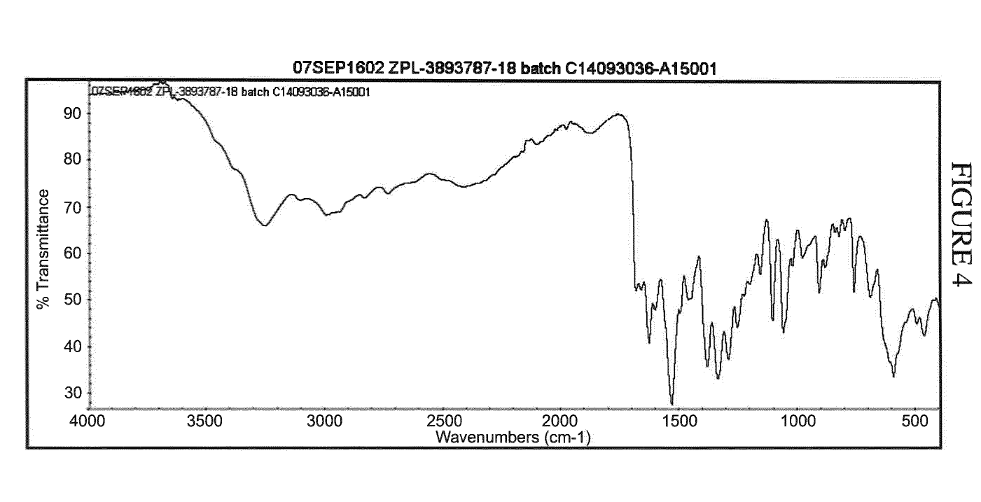

IR: (FIG. 4) IR spectrum of ultra-pure N⁴-(cyclopropylmethyl)-6-[(3R)-3-(methylamino)pyrrolidin-1-yl]pyrimidine-2,4-diamine tartrate dihydrate (Form A).

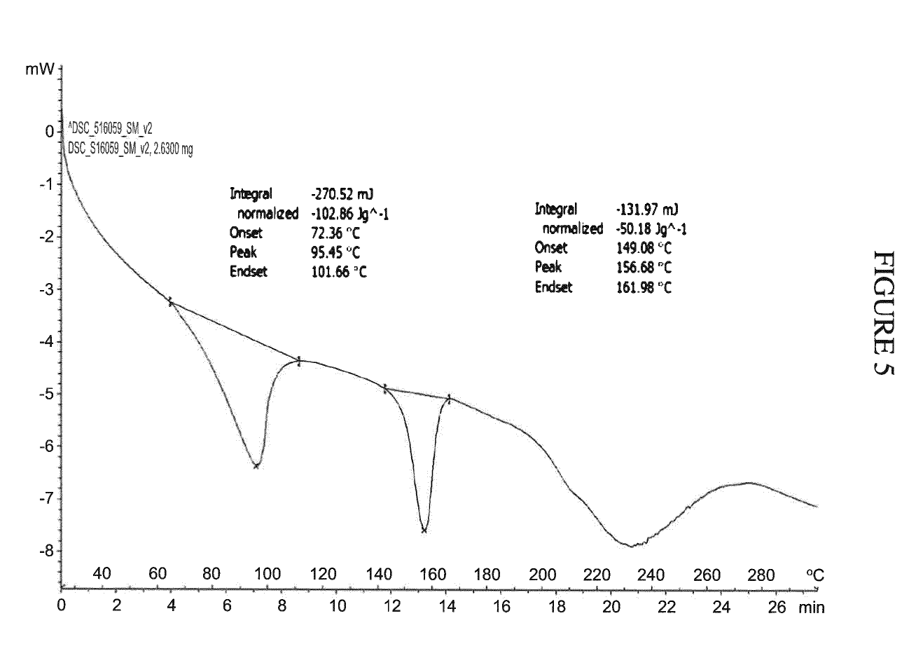

DSC Thermogram: (FIG. 5) Two endothermic events are observed at 96° C. and 153° C., corresponding to water loss and melting, respectively.

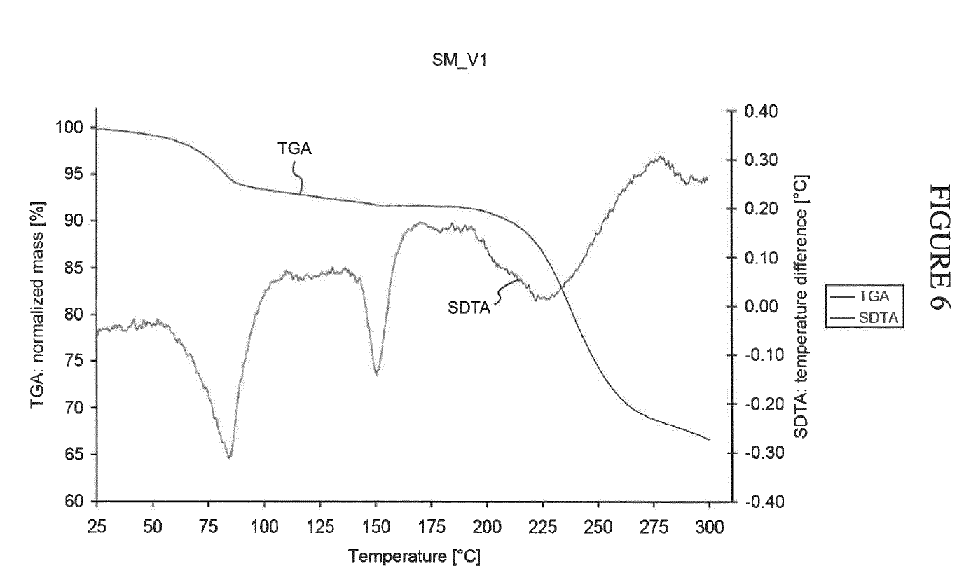

TGA/SDTA Thermogram: (FIG. 6) a mass loss of 7.9% is observed, accompanied by an endothermic event in the SDTA signal. After the water loss, the melting of the tartrate salt of ultra-pure N⁴-(cyclopropylmethyl)-6-[(3R)-3-(methylamino)pyrrolidin-1-yl]pyrimidine-2,4-diamine is observed at 147° C. Decomposition starts at about 200° C.

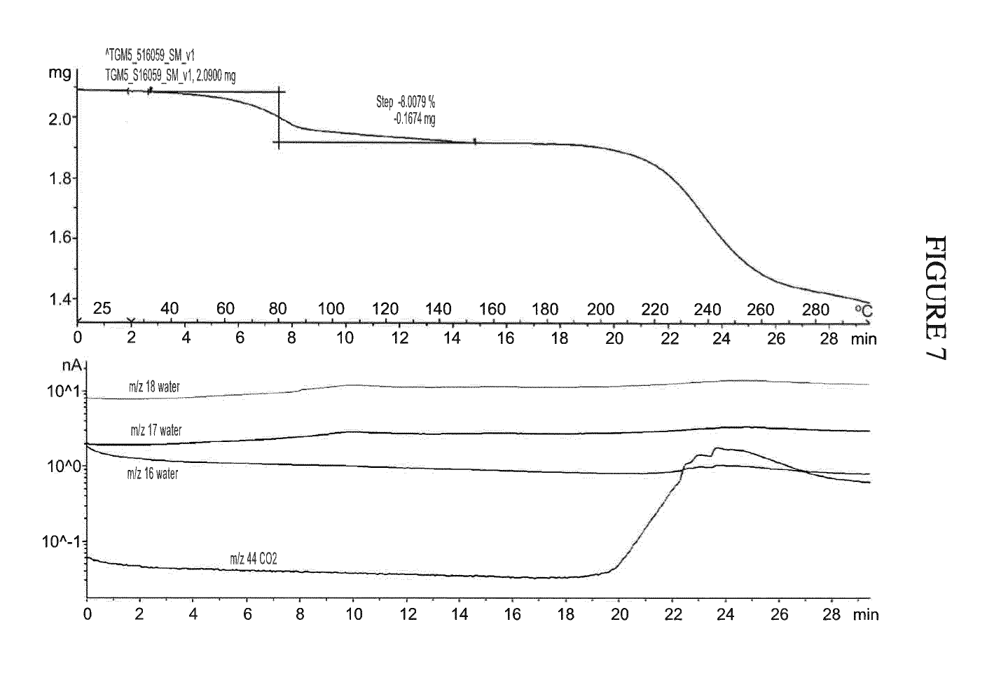

TGA/MS Thermogram: (FIG. 7) a mass loss of 7.9% is observed between 25-150° C., corresponding to two water molecules per molecule of ultra-pure N⁴-(cyclopropylmethyl)-6-[(3R)-3-(methylamino)pyrrolidin-1-yl]pyrimidine-2,4-diamine, confirming the dihydrate nature of the compound.

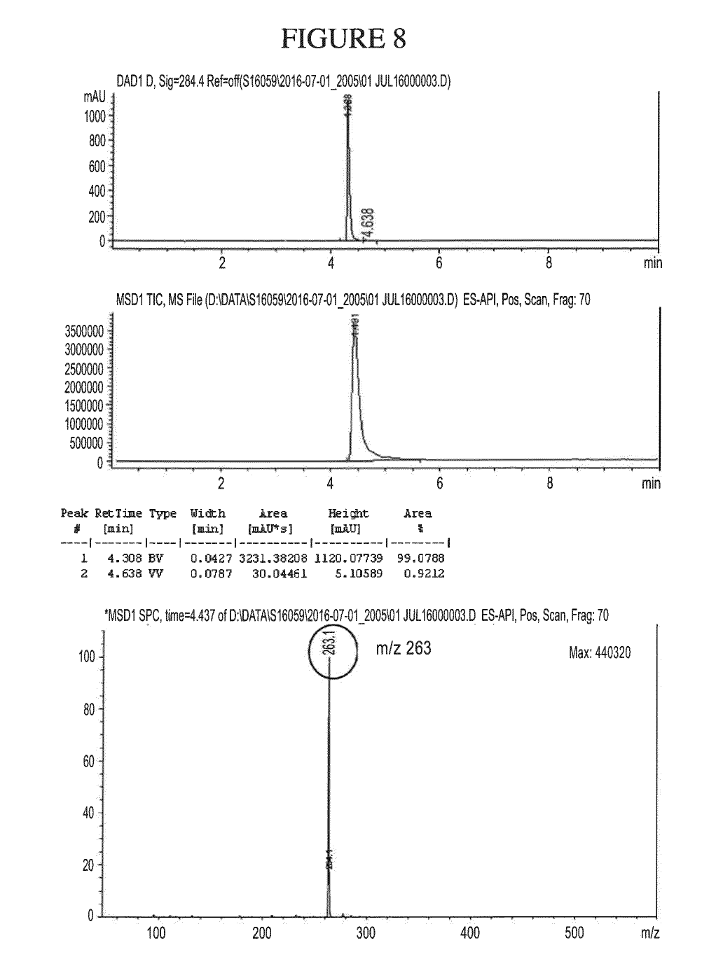

Analysis of Purity by LCMS: (FIG. 8) The purity of ultra-pure N⁴-(cyclopropylmethyl)-6-[(3R)-3-(methylamino)pyrrolidin-1-yl]pyrimidine-2,4-diamine tartrate dihydrate prepared by an analogous procedure to the one described above was assessed using LCMS. One peak in the spectrum was observed with a retention time of 4.31 minutes. This peak corresponded to 99.1% area. The peak had a m/z of 263 amu, which corresponds to the weight of N⁴-(cyclopropylmethyl)-6-[(3R)-3-(methylamino)pyrrolidin-1-yl]pyrimidine-2,4-diamine.

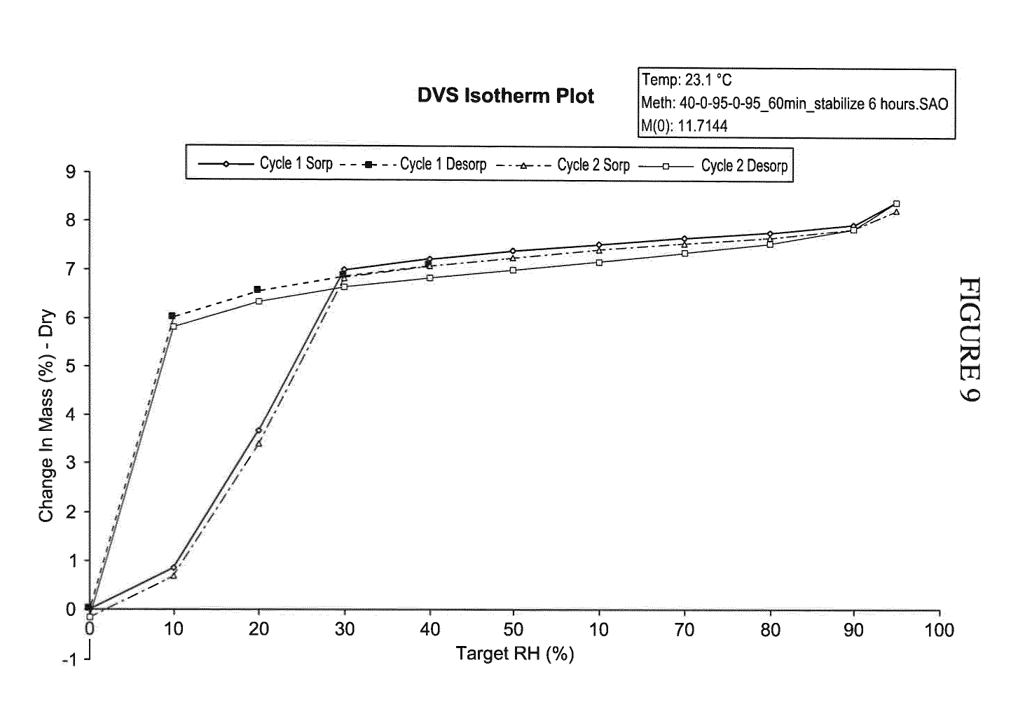

DVS Analysis: (FIG. 9) The relative humidity (RH) was cycled between 40% to 0% (desorption), up to 95% (sorption) in steps of 10%. During the desorption, the dehydration step occurs when the RH goes below 10% RH. Water uptake is observed in between 0-20% RH (1.9% mass uptake) and 20-30% (total mass uptake 6.9%). Between 30-90% RH, the weight remains stable.

### Example 5. Recrystallization of Ultra-Pure N⁴-(cyclopropylmethyl)-6-[(3R)-3-(methylamino)pyrrolidin-1-yl]pyrimidine-2,4-diamine tartrate dihydrate

Ultra-Pure N⁴-(cyclopropylmethyl)-6-[(3R)-3-(methylamino)pyrrolidin-1-yl]pyrimidine-2,4-diamine tartrate dihydrate was recrystallized from methanol/water, following a controlled cooling ramp. Filtration of the solids produced a damp cake, which is then dried, whilst controlling humidity.

### Example 6. Preparation of N⁴-(cyclopropylmethyl)-6-[(3R)-3-(methylamino)pyrrolidin-1-yl]pyrimidine-2,4-diamine gentisate

A 176 mg/mL solution of ZPL-3893787 was prepared in 1-butanol. Salt formation was performed in glass vials with a small molar excess of gentisic acid, to produce a 1:1 stoichiometry salt. The gentisic acid (647.9 mg) was weighed into 8 mL glass vials and the ZPL-3893787 solution added. Vials were incubated at room temperature and stirred for 5 days. Upon completion of the salt formation, solids were collected and dried under vacuum.

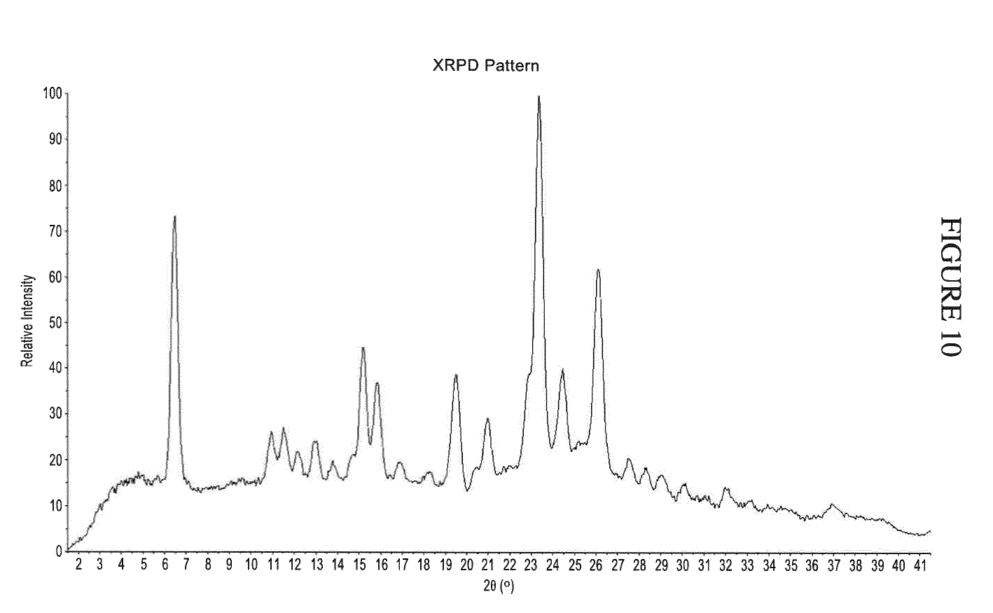

N⁴-(cyclopropylmethyl)-6-[(3R)-3-(methylamino)pyrrolidin-1-yl]pyrimidine-2,4-diamine gentisate provides a polymorph characterized by the PXRD pattern in FIG. 10.

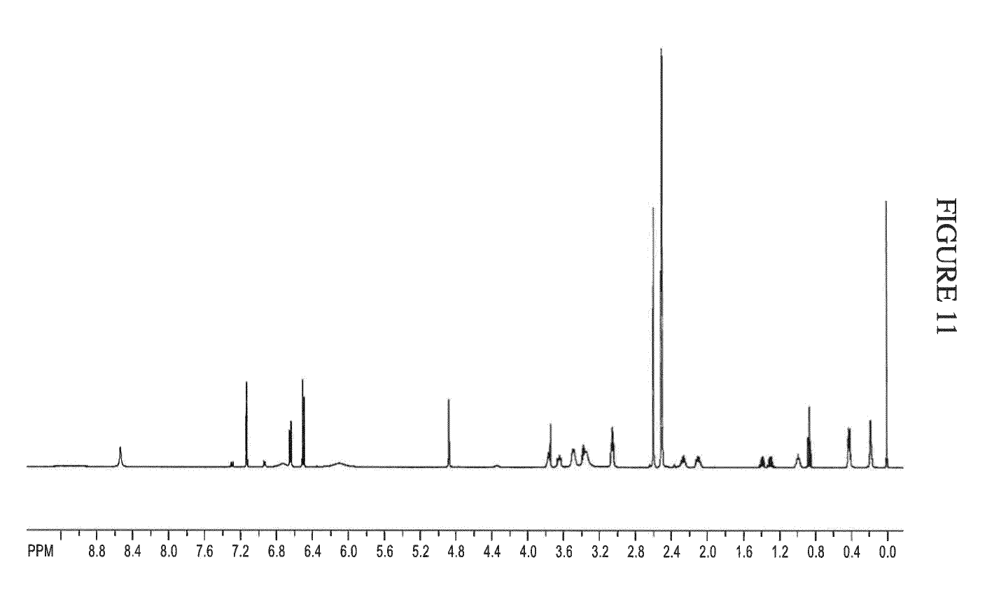

¹H NMR: (FIG. 11) ¹H NMR of N⁴-(cyclopropylmethyl)-6-[(3R)-3-(methylamino)pyrrolidin-1-yl]pyrimidine-2,4-diamine gentisate in DMSO-d₆.

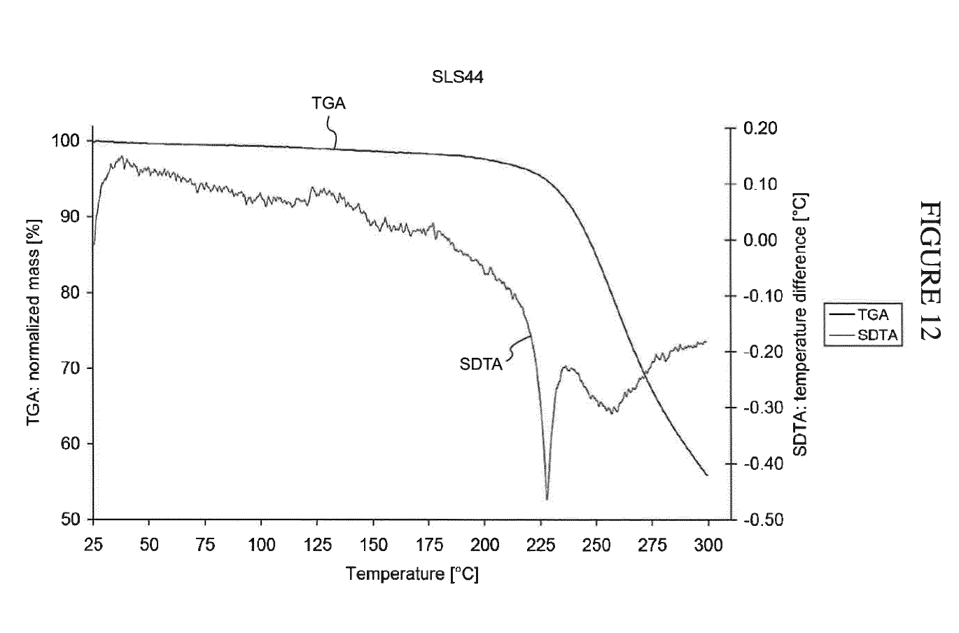

TGA Analysis (FIG. 12): A small endothermic event is observed at 134° C. (melting) and decomposition occurs at about 230° C.

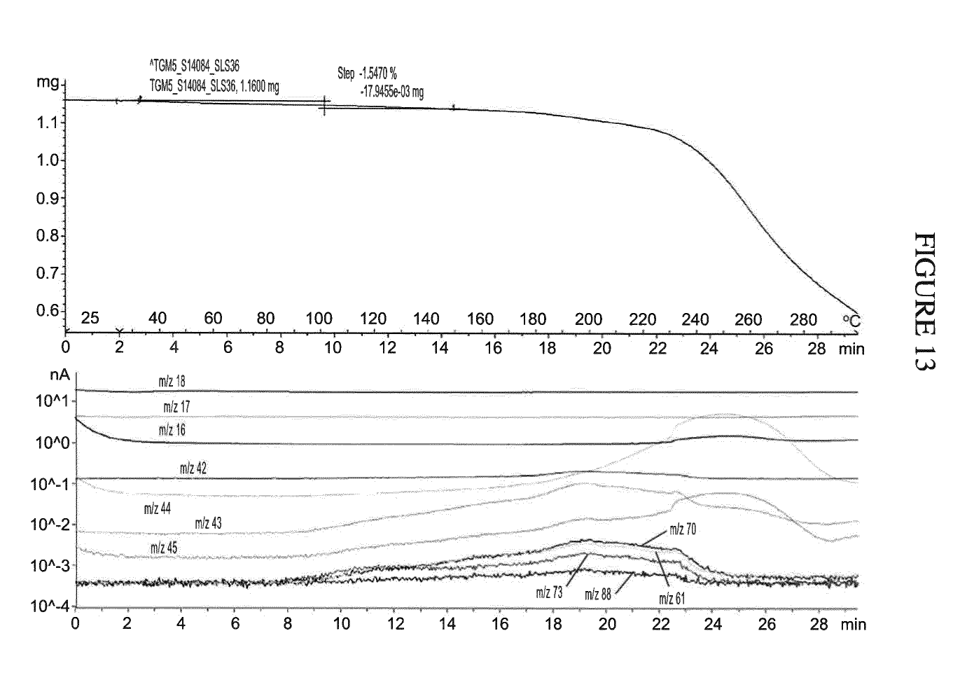

TGMS Analysis (FIG. 13): A 1.5% weight loss due to drying from water is observed at between 25 and 150° C.

### Example 7. Preparation of N⁴-(cyclopropylmethyl)-6-[(3R)-3-(methylamino)pyrrolidin-1-yl]pyrimidine-2,4-diamine salicylate

A 300 mg/mL solution of ZPL-3893787 was prepared in dichloromethane. Salt formation was performed in glass vials with a small molar excess of salicylic acid, to produce a 1:1 stoichiometry salt. The salicylic acid (580.4 mg) was weighed into 8 mL glass vials and the ZPL-3893787 solution added. Vials were incubated at room temperature and stirred for 5 days. Upon completion of the salt formation, solids were collected and dried under vacuum.

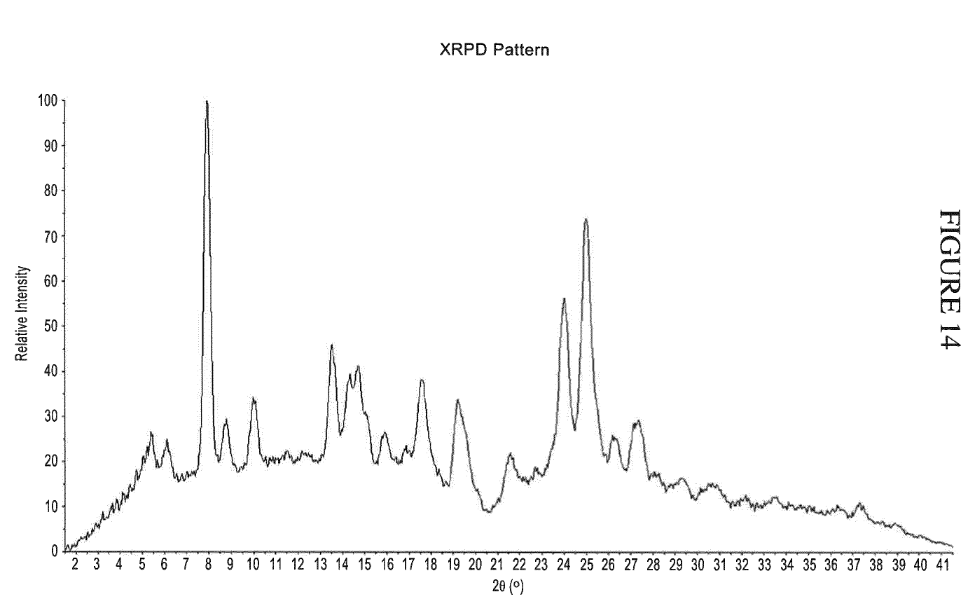

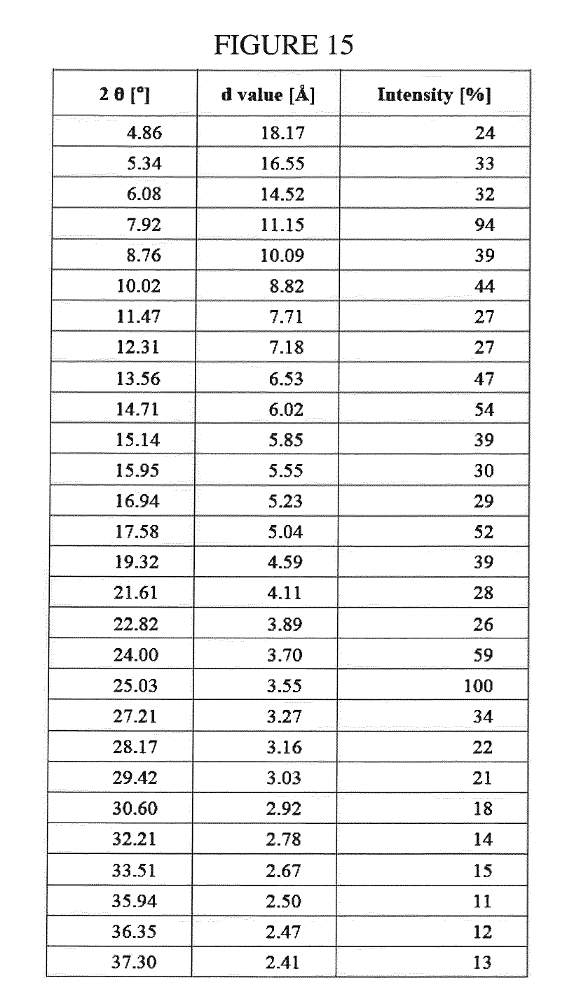

N⁴-(cyclopropylmethyl)-6-[(3R)-3-(methylamino)pyrrolidin-1-yl]pyrimidine-2,4-diamine salicylate, as prepared above provides a polymorph characterized by the PXRD pattern and PXRD peak listings in FIG. 14 and FIG. 15, respectively.

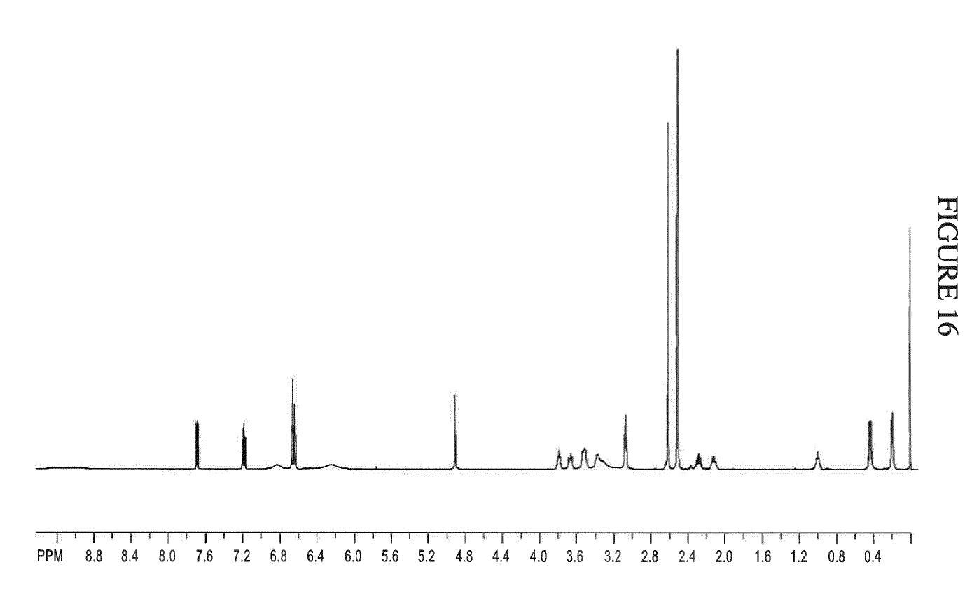

¹H NMR: (FIG. 16) ¹H NMR of N⁴-(cyclopropylmethyl)-6-[(3R)-3-(methylamino)pyrrolidin-1-yl]pyrimidine-2,4-diamine salicylate in DMSO-d₆.

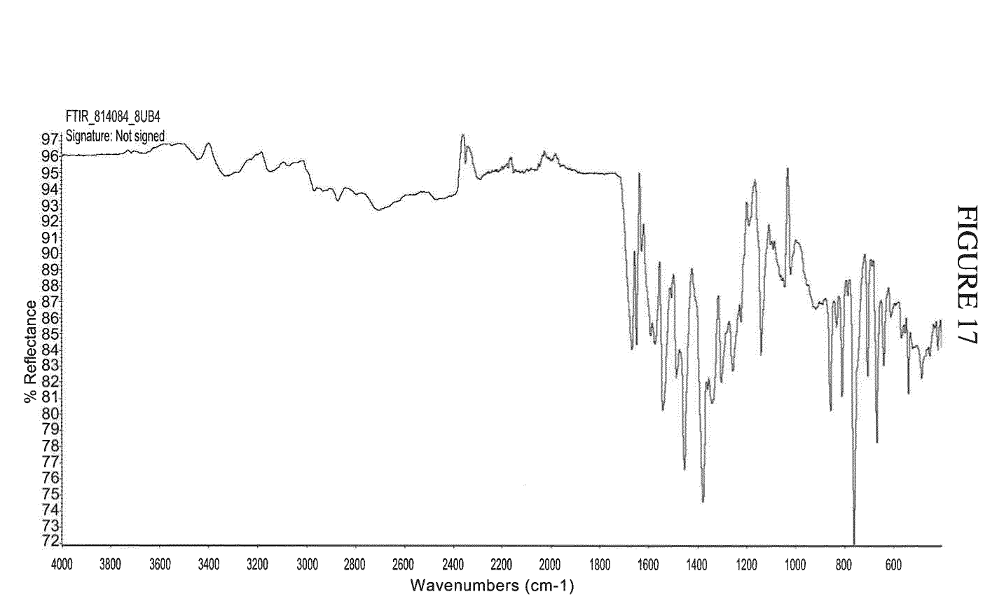

IR: (FIG. 17) IR spectrum of N⁴-(cyclopropylmethyl)-6-[(3R)-3-(methylamino)pyrrolidin-1-yl]pyrimidine-2,4-diamine salicylate.

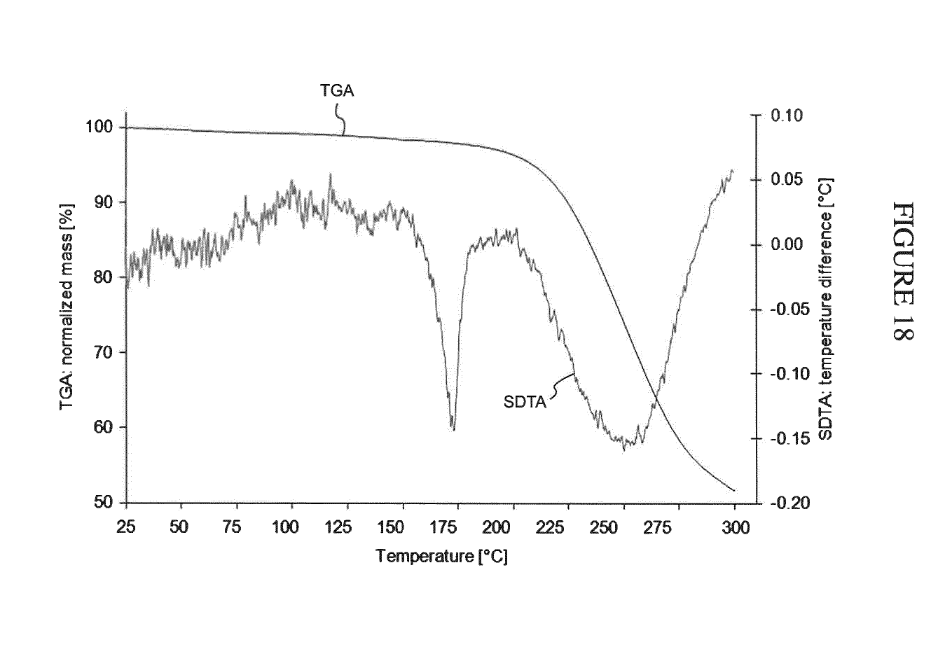

TGA Analysis: (FIG. 18): An endothermic event is observed at 173° C. (melting) and decomposition occurs at about 190° C. and above.

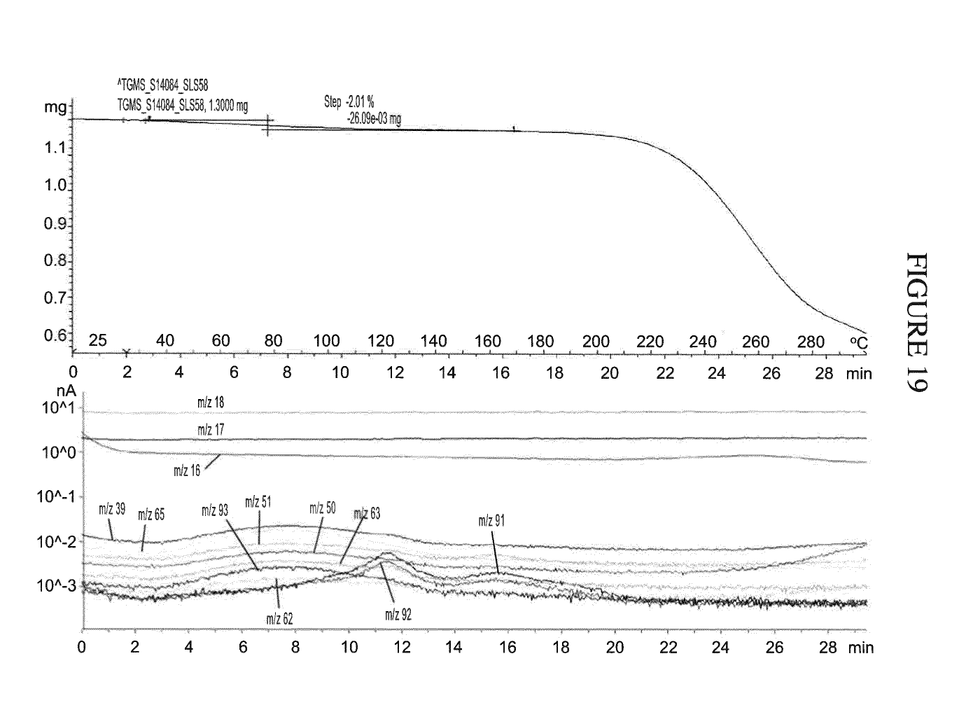

TGMS Analysis: (FIG. 19): A 1.5% weight loss due to drying from water is observed at between 25 and 160° C.

### Example 8. Preparation of N⁴-(cyclopropylmethyl)-6-[(3R)-3-(methylamino)pyrrolidin-1-yl]pyrimidine-2,4-diamine di-hydrochloride hydrate

A 176 mg/mL solution of ZPL-3893787 was prepared in 1-butanol. Salt formation was performed in glass vials with a small molar excess of hydrochloric acid, to produce a 1:2 stoichiometry salt. The hydrochloric acid (658 μL) was added to the ZPL-3893787 solution in 8 mL glass vials. Vials were incubated at room temperature and stirred for 5 days. Upon completion of the salt formation, solids were collected and dried under vacuum.

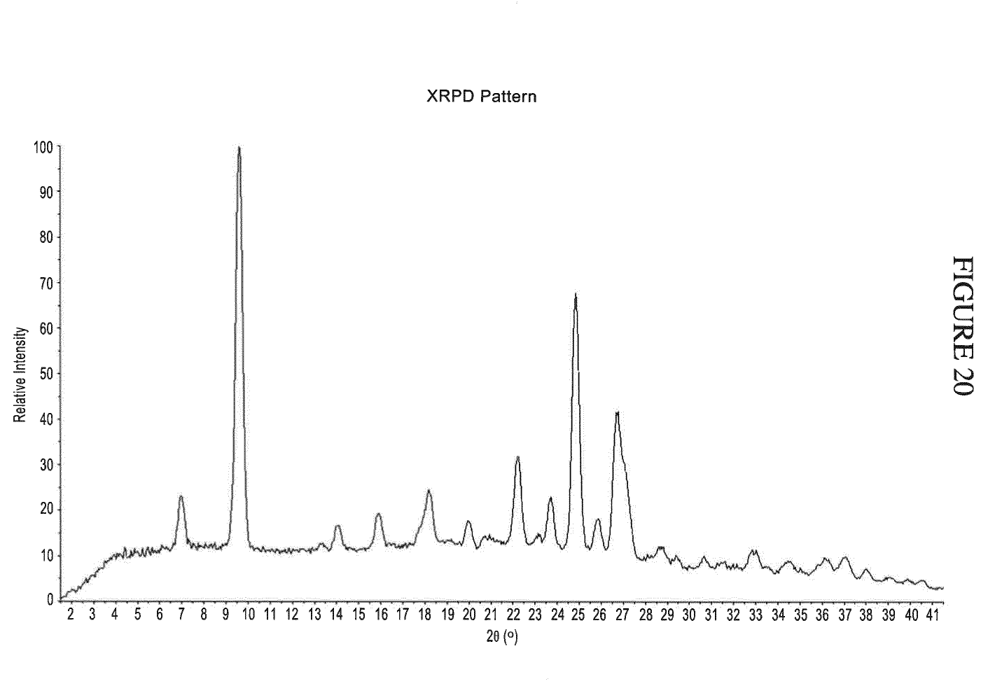

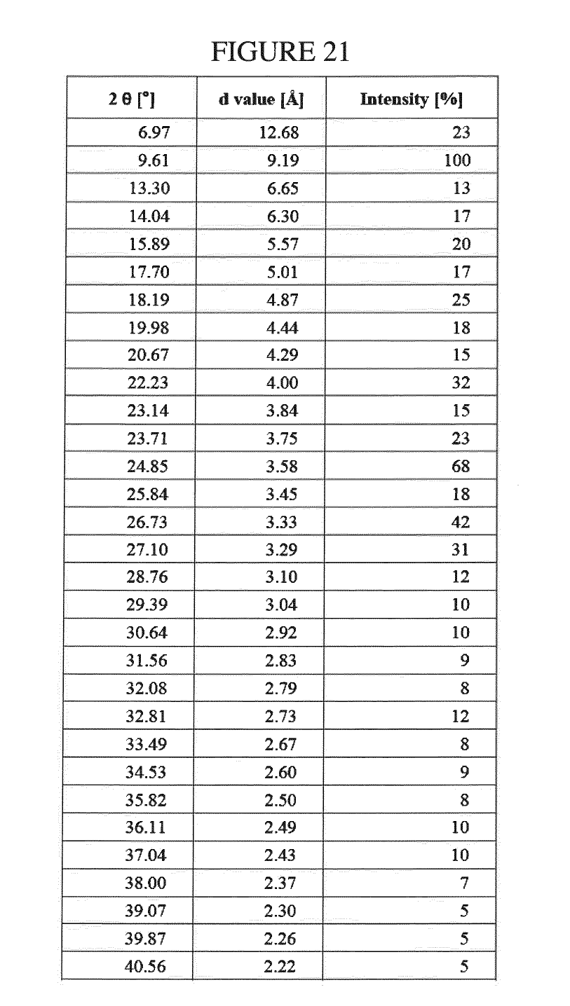

N⁴-(cyclopropylmethyl)-6-[(3R)-3-(methylamino)pyrrolidin-1-yl]pyrimidine-2,4-diamine di-hydrochloride hydrate, as prepared above provides a polymorph characterized by the PXRD pattern and PXRD peak listings in FIG. 20 and FIG. 21, respectively.

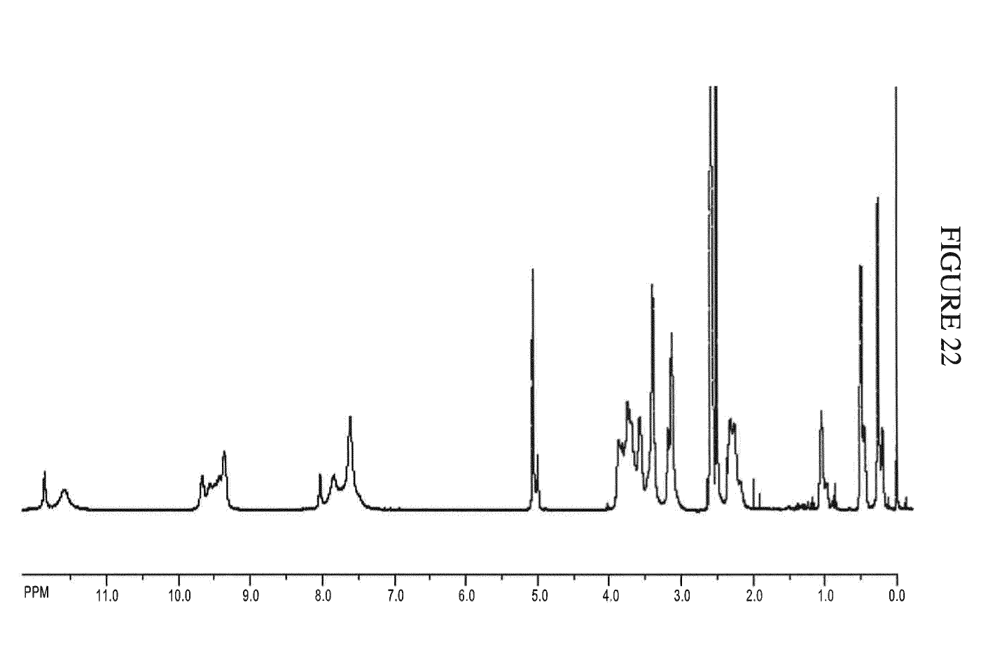

¹H NMR: (FIG. 22) ¹H NMR of N⁴-(cyclopropylmethyl)-6-[(3R)-3-(methylamino)pyrrolidin-1-yl]pyrimidine-2,4-diamine di-hydrochloride hydrate in DMSO-d₆.

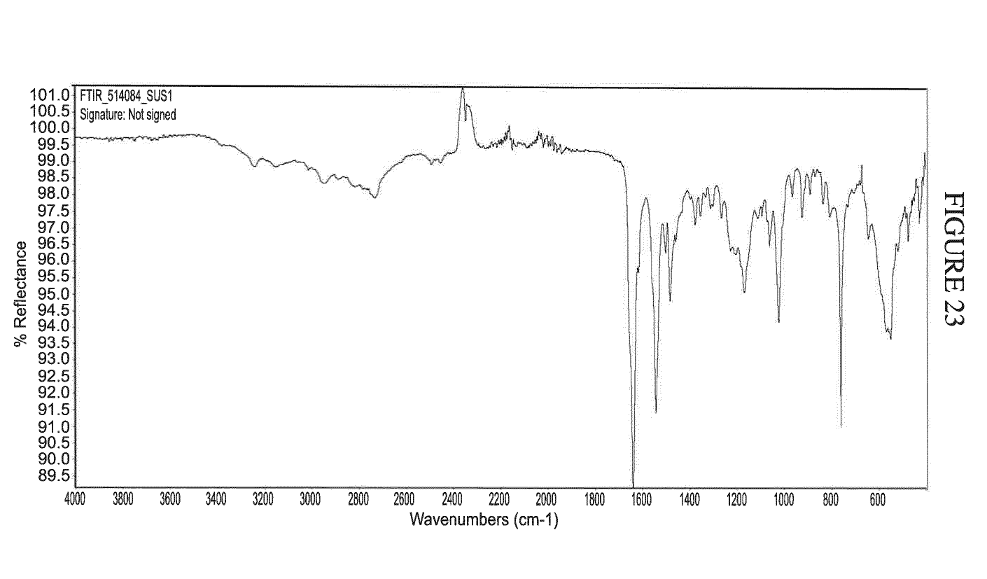

IR: (FIG. 23) IR spectrum of N⁴-(cyclopropylmethyl)-6-[(3R)-3-(methylamino)pyrrolidin-1-yl]pyrimidine-2,4-diamine di-hydrochloride hydrate.

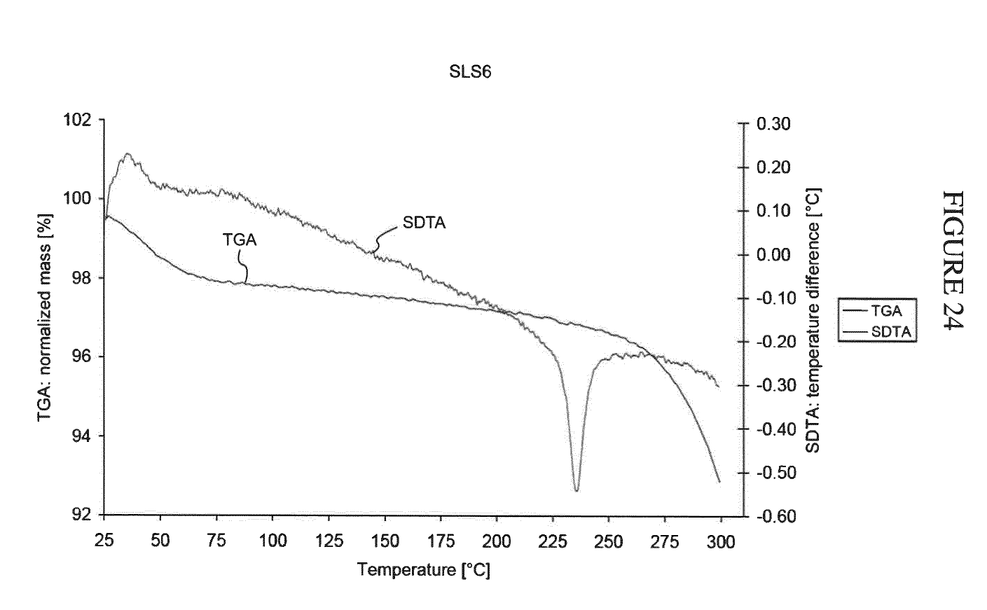

TGA/SDTA Analysis: (FIG. 24): Endothermic events are observed between 50-75° C. related to solvent loss and melting followed by decomposition occurs at 235° C.

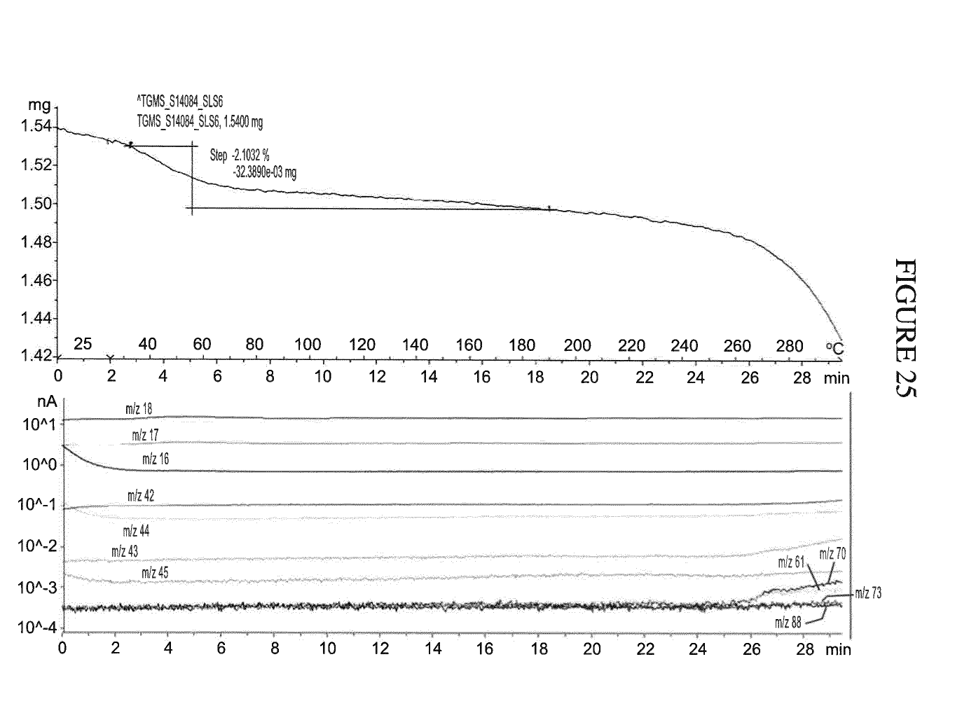

TGMS Analysis: (FIG. 25): A 1.5% weight loss due to drying from water is observed at between 25 and 160° C.

### Example 9. Preparation of N⁴-(cyclopropylmethyl)-6-[(3R)-3-(methylamino)pyrrolidin-1-yl]pyrimidine-2,4-diamine ethane disulfonate hydrate

A 300 mg/mL solution of ZPL-3893787 was prepared in dichloromethane. Salt formation was performed in glass vials with a small molar excess of 1,2,-ethanedisulfonic acid, to produce a 1:1 stoichiometry salt. The 1,2,-ethanedisulfonic acid (957.0 mg) was weighed into 8 mL glass vials and the ZPL-3893787 solution added. Vials were incubated at room temperature and stirred for 5 days. Upon completion of the salt formation, solids were collected and dried under vacuum.

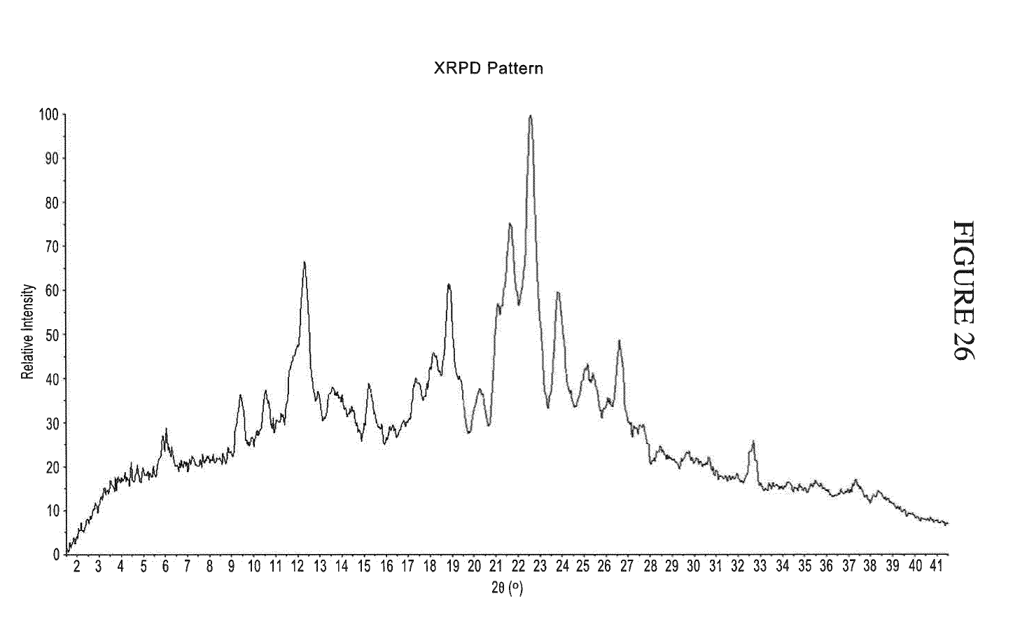

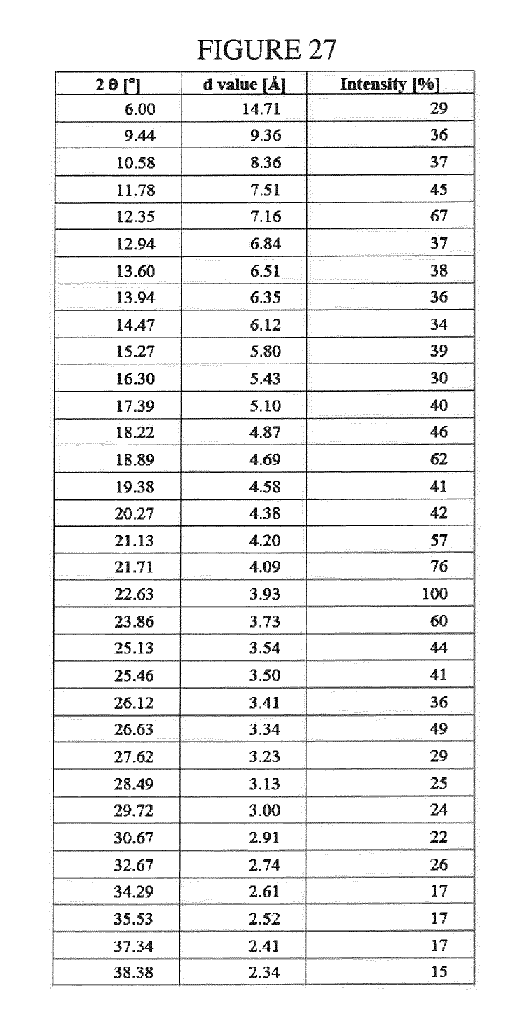

N⁴-(cyclopropylmethyl)-6-[(3R)-3-(methylamino)pyrrolidin-1-yl]pyrimidine-2,4-diamine ethane disulfonate hydrate, as prepared above provides a polymorph characterized by the PXRD pattern and PXRD peak listings in FIG. 26 and FIG. 27, respectively.

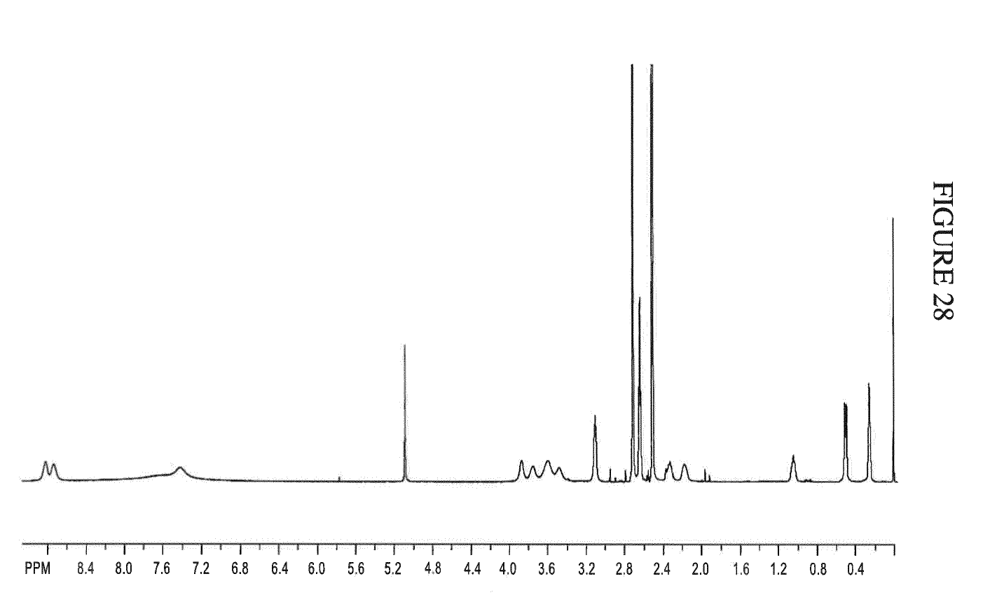

¹H NMR: (FIG. 28) ¹H NMR of N⁴-(cyclopropylmethyl)-6-[(3R)-3-(methylamino)pyrrolidin-1-yl]pyrimidine-2,4-diamine ethane disulfonate hydrate in DMSO-d₆.

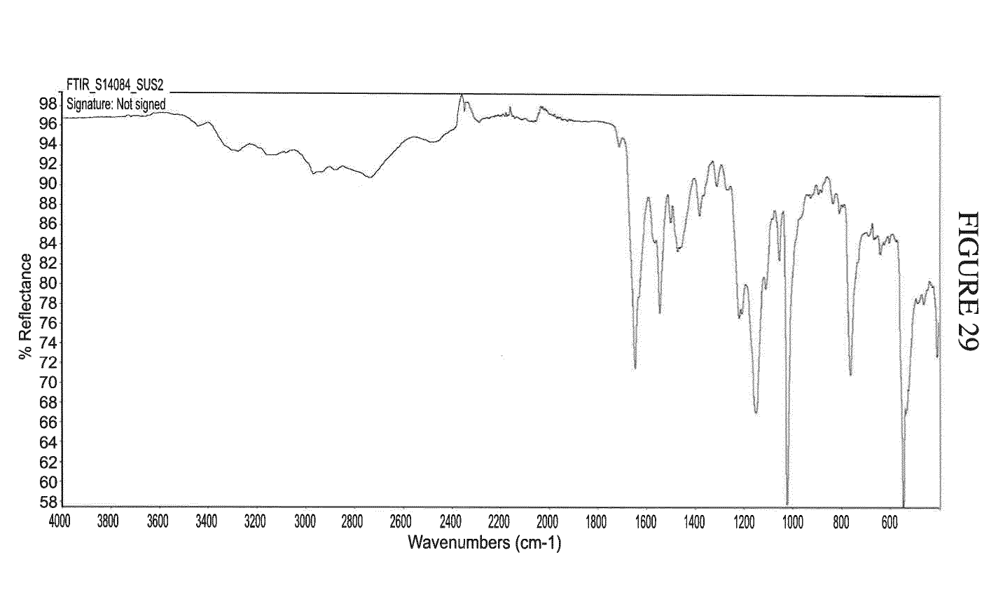

IR: (FIG. 29) IR spectrum of N⁴-(cyclopropylmethyl)-6-[(3R)-3-(methylamino)pyrrolidin-1-yl]pyrimidine-2,4-diamine ethane disulfonate hydrate.

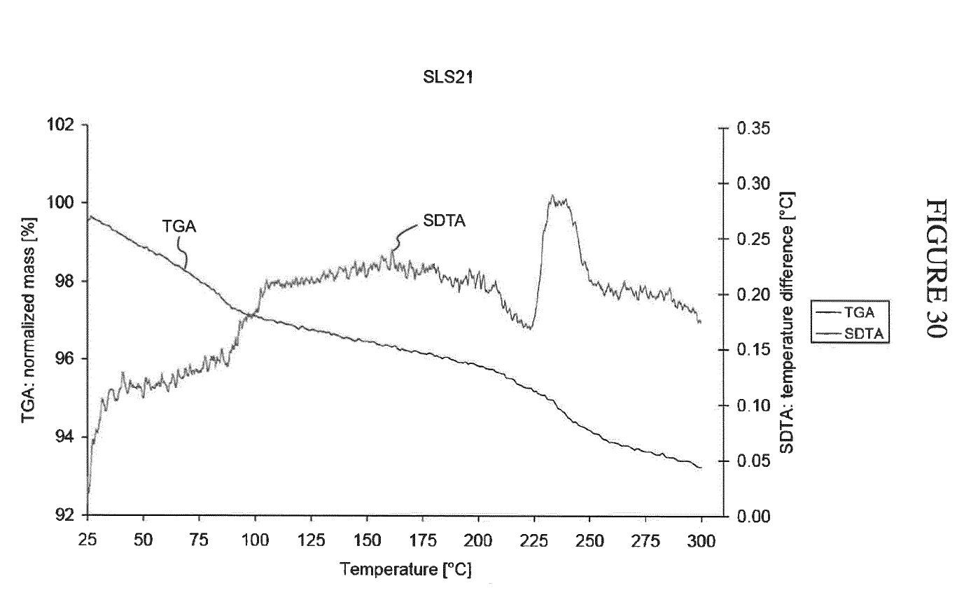

TGA/SDTA Analysis: (FIG. 30): An endothermic event is observed at 229° C., related to mass loss, melting, and decomposition.

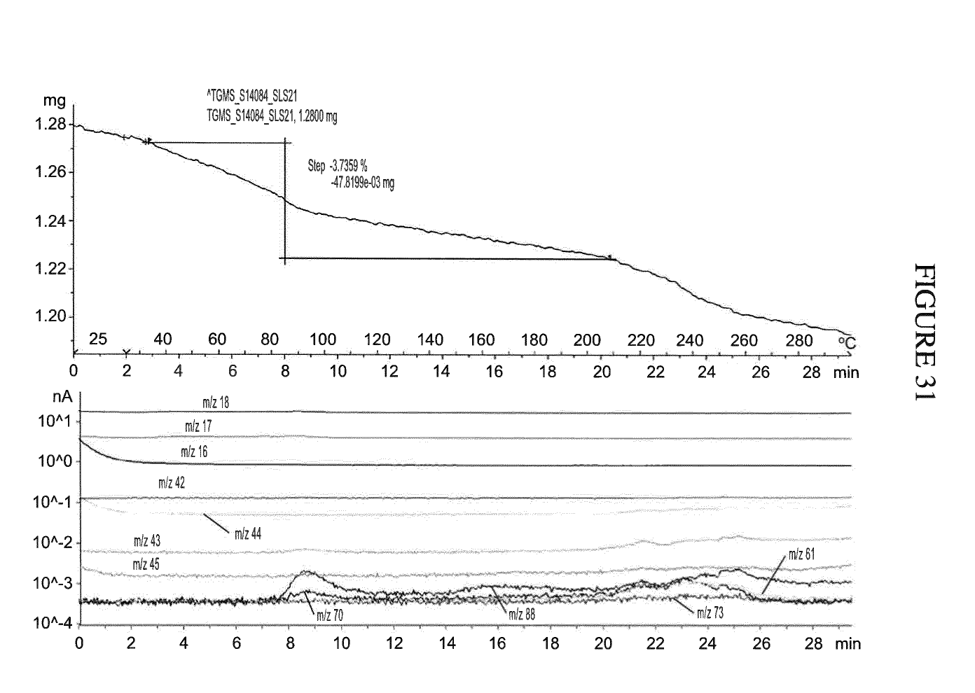

TGMS Analysis: (FIG. 31): A mass loss of 2.0% is observed prior to decomposition.

### Example 10. Phase 2a Study in Moderate to Severe Atopic Dermatitis with ZPL-389

In phase 1 trials, 62 subjects were treated with ZPL-389. Single ascending doses (0.01-48 mg) and multiple ascending doses (5, 15, and 50 mg once daily for 14 days) of ZPL-389 were safely tolerated, and a majority of observed adverse events were mild, transient, and non-dose related. Importantly, ZPL-389 was shown to be rapidly absorbed and displayed dose-proportional pharmacokinetics.

Following the phase 1 trials, a phase 2a trial was conducted to evaluate the efficacy of 8 weeks of treatment with 30 mg ZPL-389 administered once daily in adult subject with moderate to severe atopic dermatitis. The study was also designed to assess the safety and tolerability of ZPL-389 in adult subjects with moderate to severe atopic dermatitis.

This study was a randomized, double blind, placebo controlled, parallel group study of subjects having a pruritus Numerical Rating Scale (NRS) of ≥5 (0-10 scale), an Investigator's Global Assessment (IGA)≥3 (0-3 scale), and moderate-severe AD Eczema Area and Severity Index (EAST)≥12 and ≤48 (0-72 scale). In addition, subjective patient reported outcome instruments include: Ziarco Itch Diary (Electronic) Pruritus; 5D Pruritus Scale; Dermatology Life Quality Index (DLQI); and Patient Global Impression of Change (PGIG).

Each subject (aged 18-65 years) had one or more screening visits to confirm suitability to enter the study. 98 subjects with moderate to severe atopic dermatitis were randomized 2:1 to receive orally either 30 mg ZPL-389 or placebo once daily for eight weeks (56 days).

Efficacy endpoints for the trial include the following:
change from baseline in NRS for pruritus (worst itch) over 24 hours;
change from baseline in daytime NRS for pruritus (worst itch);
change from baseline in night time NRS for pruritus (worst itch);
change from baseline in NRS for sleep disturbance;
change from baseline in duration of itching;
change from baseline in verbal rating score for pruritus;
change from baseline in EASI score;
change from baseline in SCORAD score;
IGA score;
amount of rescue medication use; and/or
PGIC.

Treatment with ZPL-389 resulted in a clinically and statistically significant decrease in inflammation compared to placebo in all three efficacy tools of inflammation. Specifically, at week 8, ZPL-389 reduced EASI by 50% compared to 27% for placebo p=0.01). ZPL-389 also reduced SCORAD by 41% compared to 26% for placebo (p=0.004), and BSA (Body Surface Area) affected by inflammation change from baseline was −18% active compared to −12% placebo (p=0.044). Additionally, IGA was reduced by 36% compared to 25% for placebo, and 19% of subjects had clear or almost clear skin (as evidenced by IGA response and remission have odds rations 2.51 and 2.71).

Throughout the study, ZPL-389 was found to be well tolerated with a favorable safety profile comparable to placebo, and no differences in Treatment Emergent Adverse Events (TEAE) were observed between ZPL-389 and placebo. The 7 withdrawals in the study due to lack of efficacy were all in the placebo group. Moreover, rescue medication use was also lower in patients receiving ZPL-389.

Thus, ZPL-389 showed a clinically and statistically significant reduction in the signs of moderate to severe atopic dermatitis in adults, as evidenced by three separate efficacy tools (EASI, IGA, and SCORAD). Moreover, pruritus was markedly reduced, and the magnitude of reduction in pruritus seen in this study is clinically meaningful, and is statistically significant compared to baseline.

Accordingly, ZPL-389 is the first histamine H4 antagonist shown to significantly reduce inflammation in moderate to severe atopic dermatitis.

### Example 11. Initial Formulation of a Tablet Containing ZPL-389 by Dry Granulation

Dry granulation was performed at a 60 g scale. The dry granulation formulation shown in Table 5 was not easily formed into slugs due to poor flow of the blend, which required intervention during slugging and manual compressing to form the slugs. The slugs that were formed were good quality and provided a robust compact, which could be handled without breaking easily. Slugs were compressed using 12 mm flat faced tooling, had a thickness of 4 to 5 mm, and were milled using a Comil with a 991 μm screen.

Granules had a better flow than the blend which improved the flow of the material through the hopper when compressing the final tablet (36% versus 31% Carr's index for blend and granules, respectively). The tablets were compressed using 8.5 mm normal round concave tooling and hardness was targeted at 65, 85, and 105N. Tablets were of a suitable thickness and hardness. See Table 6. The force/hardness curve was steep for this batch with changes of 0.5 on the F-press leading to large variations in hardness. The slugs were probably slightly harder than required but the intra-granular blend is suitable for roller compaction, which would control the porosity of the ribbons well and would be more consistent than slugging as a process.

Disintegration times for the dry granulation were faster than for the wet granulation batch and a relationship between hardness and disintegration time was observed.

TABLE 5
ZPL-389 Starting Point: Dry Granulation Formulation

| Ingredient | % w/w | Amount (mg) |
|---|---|---|
| P717 | 28 | 56.0 |
| Microcrystalline cellulose | 47.4 | 94.8 |
| DCP | 18.6 | 37.2 |
| Croscarmellose sodium | 5 | 10 |
| Magnesium stearate | 1* | 2 |
| Total | 100 | 200 |

*0.5% intra-granular, 0.5% extragranular (P717 = ZPL-389)

TABLE 6
Tablet Data

| Target | Force | | | | Disintegration |
|---|---|---|---|---|---|
| hardness | setting | Weight | Hardness | Thickness | time |
| (N) | on press | (mg) | (N) | (mm) | (NMT minutes) |
| 65 N | 22.5 | 202.8 | 65.2 | 4.41 | 3 |
| 85 N start | 23.15 | 197.8 | 77.2 | 3.576 | 5 |
| 85 N End | 23 | 198 | 44.6 | 3.576 | N/A |
| 105 N | 23 | 196 | 78.8 | 3.58 | 8 |

### Example 12. Initial Formulation of a Tablet Containing ZPL-389 by Wet Granulation

The formulation shown in Table 7 was produced at small scale (40 g) using water as a granulating liquid. The wet granulation process was rapid for the formulation shown below, with 6 mL (15%) of water contributing to a slightly over granulation wet mass. Initial moisture content of the blend was 5.25%, the moisture content of the dried granules was 2.77%. The drying was not controlled at this stage (60 minutes in oven at 60° C.), but a target moisture of initial ±0.5% (w/w) would be used for larger batches.

TABLE 7
ZPL-389 Starting Point: Wet Granulation Formulation

| Ingredient | % w/w | Amount (mg) |
|---|---|---|
| P717 | 56 | 56 |
| Mannitol | 23.8 | 23.8 |
| DCP | 10.2 | 10.2 |
| Sodium Starch glycolate | 5 | 5 |
| HPC EXF | 4 | 4 |
| Magnesium stearate | 1 | 1 |
| Total | 100 | 100 |

(P717 = ZPL-389, DCP = dicalcium phosphate anhydrous, HPC = hydroxypropyl cellulose)

The dried granules were screened through a 1000 μm screen with and without brushing. Without brushing, approximately 50% of the granules went through the screen and with brushing, the remaining material passed through except for 4.29 g of hard lumps. The loss may be attributed to a slight over granulation. The granules were lubricated and compressed using a 5 mm normal round concave (nrc) tool set.

Hardness values of 25N, 50N, and 75N were targeted leading to a compression profile for the batch. See Table 8. The tablets were slightly thicker than expected and a 6 mm tool set would have been more suitable.

Disintegration times were less than 15 minutes, and a relationship between hardness and disintegration was observed.

TABLE 8
Tablet Data

| Target | Force | | | | Disintegration |
|---|---|---|---|---|---|
| hardness | setting on | Weight | Hardness | Thickness | time |
| (N) | press | (mg) | (N) | (mm) | (NMT minutes) |
| 25 N | 24 | 101.8 | 23 | 4.41 | 8 |
| 50 N start | 24.75 | 103 | 55.4 | 4.26 | 11 |
| 50 N End | 24.75 | 97 | 44.6 | 4.272 | N/A |
| 75 N | 28 | 100 | 77.6 | 4.13 | 13 |

### Example 13. Formulation of a Tablet Containing ZPL-389 by Direct Compression

A 30 mg direct compression blend was prepared at a 100 g scale. As the flow of the dry granulation formulation was poor and could not be compressed into tablets, the formulation was reformulated to a 10 mg dose. See Table 9.

TABLE 9
Direct Compression Formulation of ZPL-389

| Ingredient | % w/w | Amount (mg) |
|---|---|---|
| P717 | 9.335 | 18.67 |
| Mannitol 200SD | 84.665 | 169.33 |
| Croscarmellose sodium | 5 | 10 |
| magnesium stearate | 1 | 2 |
| Total | 100 | 200 |

The direct compression formulation at 10 mg dose was performed at a 40 g batch size. By reducing the API loading, it was expected that the flow would improve. The direct compression formulation at the 10 mg dose of ZPL-389 displayed good flow properties (Carr's index of 23%) and flowed well enough to be compressed using 8.5 mm nrc tooling. The tablets disintegrated from the center forming a ring, and disintegration times were very fast. Tablet data is shown below in Table 10.

TABLE 10
Tablet Data

| | Force | | | | Disintegration |
|---|---|---|---|---|---|
| Target | setting | Weight | Hardness | Thickness | time |
| hardness (N) | on press | (mg) | (N) | (mm) | (NMT minutes) |
| 65 N | 23.75 | 195.6 | 55.6 | 3.952 | 1 |
| 85 N start | 24.5 | 203 | 90.8 | 3.91 | 1 |
| 85 N End | 24.5 | 202 | 80.4 | 3.91 | N/A |
| 105 N | 25.25 | 203.6 | 117.8 | 3.814 | 2 |

### Example 14. Formulation of a Tablet Containing ZPL-389 by Moisture Activated Dry Granulation

The moisture activated dry granulation formulation of ZPL-389, which was produced at a 40 gram batch size, is shown in Table 11.

TABLE 11
Moisture Activated Dry Granulation Formulation of ZPL-389

| | | |
|---|---|---|
| Ingredient | % w/w | Amount (mg) |

Moisture activated

| | | |
|---|---|---|
| P717 | 22.4 | 56.0 |
| DCP | 19.28 | 48.2 |
| Povidone K12 | 4 | 10.0 |

Extra-granular

| | | |
|---|---|---|
| Microcrystalline cellulose | 47.32 | 118.3 |
| PH200 | | |
| Croscarmellose sodium | 5 | 12.5 |
| Silicon dioxide | 1 | 2.5 |
| Magnesium stearate | 1 | 2.5 |

The intra-granular material was activated with 0.5 mL of water and granulation was very short. Initial moisture was 4.81% by weight, but after blending the granulate with the extra-granular material the blend moisture was 2.59% by weight. The flow of the granulate by Carr's index was 27% for the granules and the final lubricated blend. Acceptable tablets were compressed using 10 mm nrc tooling. Only a few tablets were compressed at the middle hardness of 100N, with low and high hardness of 80N and 120N also targeted. The force setting when adjusted by 0.1 led to wide variation in hardness. The tablets disintegrated from the center forming a ring. Disintegration times were rapid. Tablet data is shown below in Table 12.

TABLE 12
Tablet Data

| | Force | | | | Disintegration |
|---|---|---|---|---|---|
| Target | setting | Weight | Hardness | Thickness | time |
| hardness (N) | on press | (mg) | (N) | (mm) | (NMT minutes) |
| 80 N | 27.75 | 261.2 | 77.8 | 4.41 | 1 |
| 100 N start | 27.85 | 258.2 | 117.8 | 3.778 | 2 |
| 120 N | 28.05 | 259.4 | 138.6 | 3.766 | 3 |

### Example 15. Modified Formulation of a Tablet Containing ZPL-389 by Dry Granulation

Modification of the initial dry granulation described in Example 11, supra, was tested for improved processability, to establish a process to manufacture a powder or granulation of ZPL-389 with consistent API uniformity and a compression profile for the desired tablet size, and to demonstrate that the formulation process is reproducible. For example, the formulation was modified to remove dicalcium phosphate anhydrous (DCP) from the intra-granular portion and to increase the level of disintegrant from 5% to 8% by weight, i.e., (w/w).

In addition, a non-functional coat (e.g., Opadry II) can be applied to the tablets.

The aim is to establish a coated tablet formulation with the dose strength of 3 mg, 10 mg, and 30 mg. It is expected that a tablet having suitable compression properties, good granule flow, good tablet properties, and fast disintegration and dissolution will be most desirable.

There was no difference in preparing the intra-granular material for dry granulation formulation other than the formulation changes illustrated in Table 13.

TABLE 13
Dry Granulation Modified Formulation

| | Percentage | | |
|---|---|---|---|
| | Formula | Final Unit | Batch |
| Material | (% w/w) | Dosage (mg) | Quantity (g) |
| P717 | 25.75 | 51.5 | 15.45 |
| Avicel PH200 UM | 47.4 | 94.8 | 28.44 |
| Croscarmellose sodium | 4 | 8 | 2.4 |
| Magnesium stearate | 0.5 | 1 | 0.3 |
| Intra-granular material Total | 77.65 | 155.3 | 46.59 |
| (E) | | | |
| Extra-granular material | | | |
| DCP | 17.85 | 35.7 | 10.71 |
| Croscarmellose sodium | 4 | 8 | 2.4 |
| magnesium stearate | 0.5 | 1 | 0.3 |
| Total | 100 | 200 | 60 |

Because the flow of the blend was still poor from the hopper into the die, this transfer was performed manually. A number of slugs were produced during setup which resulted in poor yield. However, the slugs broke easily when screened through a 1 mm sieve using a mortar, as the slugs were too thick to brush. Granules were then mixed with the extra granular material and lubricated, resulting in a Carr's index of 27%. Compression of the tablets at mid hardness using an 8.5 mm normal round concave tool set could only be achieved due to poor yield from the slugging process. While target hardness of 85 N could be achieved using a force setting of 30 but there was some variability, the weight of the tablet did reduce throughout the run suggesting that there was some segregation. Even with the poor flow from Carr's index, there were no issues in filling the die. Force setting changes of 0.25 resulted in the hardness changing significantly. Tablet data is presented in Table 14.

TABLE 14
Tablet Data

| Target | Force | | | | Disintegration |
|---|---|---|---|---|---|
| hardness | setting on | Weight | Hardness | Thickness | time |
| (N) | press | (mg) | (N) | (mm) | (NMT minutes) |
| 65 N | 30 | 209 | 61 | 3.89 | N/A |
| 85 N | 30.25 | 198.6 | 80.8 | 3.81 | N/A* |
| 105 N | 31.5 | 200 | 105 | 3.67 | N/A |

### Example 16. Modified Formulation of a Tablet Containing ZPL-389 by Wet Granulation

As with the dry granulation formulation, the formulation of the tablet by wet granulation (see Example 12, supra) was modified and tested for improved processability. See Table 15. For example, because the endpoint using mannitol in the formulation was too sudden, mannitol was replaced with microcrystalline cellulose.

The intra-granular material for the wet-granulation formulation accounted for 90% fill of the Kenwood chopper bowl, upon blending using high shear this volume reduced to 60%. The blend was initially white in appearance but, after granulation, the blend was off-white to yellow. 10 mL of water was added drop wise to the blend while mixing, which resulted in a sudden change in end point. The microcrystalline cellulose (MCC), e.g., Avicel PH200, formulation performed better than the mannitol formulation, as the MCC formulation did not appear to be as over granulated as the mannitol formulation.

TABLE 15
Wet Granulation Modified Formulation

| | Percentage | Final Unit | Batch |
|---|---|---|---|
| Material | Formula (% w/w) | Dosage (mg) | Quantity (g) |
| P717 | 51.5 | 51.5 | 30.9 |
| Avicel PH200 | 19.75 | 19.75 | 11.85 |
| DCP | 19.75 | 19.75 | 11.85 |
| sodium starch glycolate | 5 | 5 | 3 |
| HPC EXF | 3 | 3 | 1.8 |
| Magnesium stearate | 1 | 1 | 0.6 |
| Purified water | q.s. | N/A | 5-10 |
| Total | 100 | 100 | 60 |

The granules were dried in an oven at 60° C. and the moisture was within the specification so no over drying had occurred. A few hard lumps in the granules were observed, but these could be reduced with a mortar and pestle, resulting in a 97% yield of granules.

The granules were lubricated and compressed at the middle hardness target of 60N using 6 mm tooling. While the force setting was 22.25 for 60N hardness, a range of 22-25 resulted in a hardness range of 55 to 115N. The flow of the lubricated granules was very good at 14% on the Carr's index. Tablet Data is shown in Table 16.

TABLE 16
Tablet Data

| | | | | | Disintegration |
|---|---|---|---|---|---|
| Target | Force | | | | time |
| hardness | setting on | Weight | Hardness | Thickness | (NMT |
| (N) | press | (mg) | (N) | (mm) | minutes)* |
| 25 N | 20 | 106 | 27 | 3.22 | N/A |
| 50 N | 22.25 | 112.8 | 60.2 | 3.38 | N/A |
| 75 N | 25 | 111 | 115 | 2.91 | N/A |

*not yet performed

### Example 17. Dissolution Method Establishment

Dissolution method establishment of the formulations of ZPL-389 was performed in two separate media: 0.01 M HCl (pH=2) and pH 6.8 buffer. These data were compared to the existing dosage form of ZPL-389. Both the initial dry and wet granulation formulations (see Examples 11 and 12, supra) did not result in complete dissolution after 60 minutes at 50 rpm, which suggested that the ZPL-389 was being retarded on release.

When the speed of the dissolution method was increased to 75 rpm, dissolution was still not complete at 60 minutes and there was some variability between the tablet release profiles. This variability is mainly due to differences in the formulation and processing parameters, e.g., the wet granulation formulation was slightly over-granulated whereas the dry granulation formulation was compressed slightly harder than required during slugging.

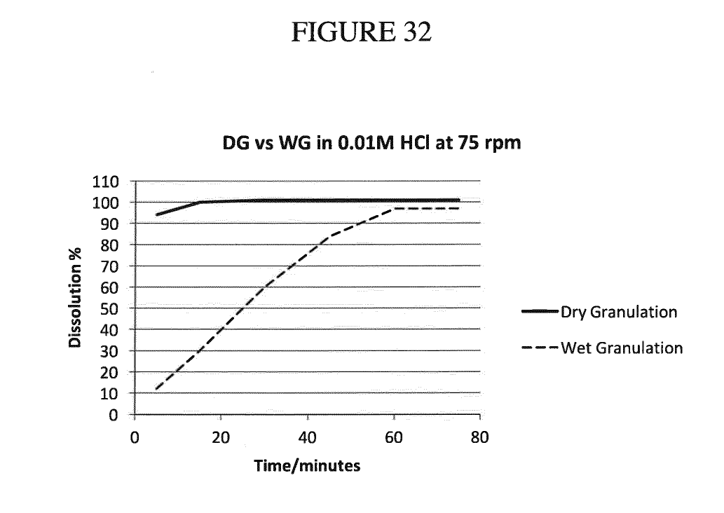

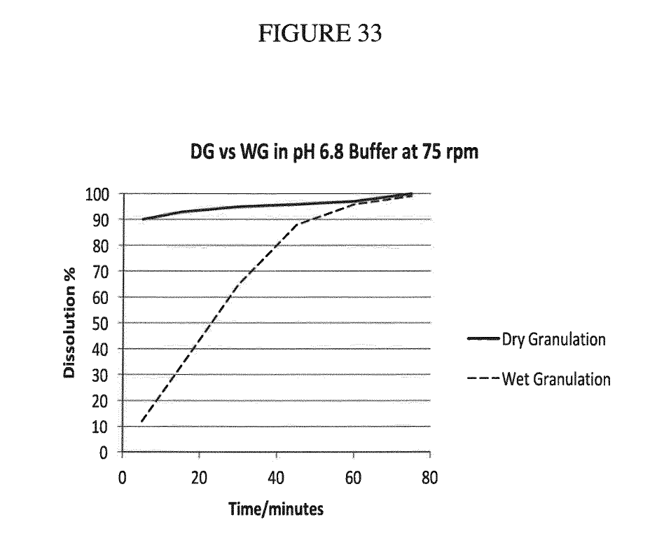

Additional dissolution experiments were conducted on the modified 30 mg dry and wet granulation formulation tablets (see Examples 15 and 16, supra) in 0.01 M HCl and pH 6.8 buffer, both at 75 rpm. The results of these experiments are summarized in FIGS. 32 and 33. The dry granulation (DG) tablet formulation releases more rapidly than the wet granulation (WG) tablet formulation, in both 0.01 M HCl (FIG. 32) and pH 6.8 buffer (FIG. 33).

### EQUIVALENTS

The application can be embodied in other specific forms without departing from the spirit or essential characteristics thereof. The foregoing embodiments are therefore to be considered in all respects illustrative rather than limiting on the application described herein. Scope of the application is thus indicated by the appended claims rather than by the foregoing description, and all changes that come within the meaning and range of equivalency of the claims are intended to be embraced therein.

1-200. (canceled)

201. A composition comprising N⁴-(cyclopropylmethyl)-6-[(3R)-3-(methylamino)pyrrolidin-1-yl]pyrimidine-2,4-diamine tartrate dihydrate, wherein the composition is at least 98% pure.

202. A composition comprising N⁴-(cyclopropylmethyl)-6-[(3R)-3-(methylamino)pyrrolidin-1-yl]pyrimidine-2,4-diamine tartrate dihydrate, wherein the composition further comprises less than 1% of 4-N-butyl-6-[(3-(methylamino)pyrrolidin-1-yl]pyrimidine-2,4-diamine.

203. The composition of claim 202, wherein the 4-N-butyl-6-[(3-(methylamino)pyrrolidin-1-yl]pyrimidine-2,4-diamine is 4-N-butyl-6-[(3R)-3-methylamino)pyrrolidin-1-yl]pyrimidine-2,4-diamine.

204. The composition of claim 202, wherein the composition further comprises less than 0.95%, 0.90%, 0.85%, 0.80%, 0.75%, 0.70%, 0.65%, 0.60%, 0.55%, 0.50%, 0.45%, 0.40%, 0.35%, 030%, 0.29%, 0.28%, 0.27%, 0.26%, 025%, 0.24%, 0.23%, 0.22%, 0.21%, 0.20%, 0.15%, 0.10%, or 0.05% of 4-N-butyl-6-[(3-(methylamino)pyrrolidin-1-yl]pyrimidine-2,4-diamine.

205. The composition of claim 204, wherein the composition comprises less than 0.26% of the impurity.

206. The composition of claim 201, wherein the composition comprises a polymorph of N4-(cyclopropylmethyl)-6-[(3R)-3-(methylamino)pyrrolidin-1-yl]pyrimidine-2,4-diamine tartrate dihydrate distinguished by PXRD peaks at about 6.7, 9.2, 22.4, and 24.4 degrees 2-theta.

207. The composition of claim 202, wherein the composition comprises a polymorph of N4-(cyclopropylmethyl)-6-[(3R)-3-(methylamino)pyrolidin-1-yl]pyrimidine-2,4-diamine tartrate dihydrate distinguished by PXRD peaks at about 6.7, 9.2, 22.4, and 24.4 degrees 2-theta.

208. The composition of claim 206 wherein the polymorph comprises two additional peaks at about 13.5 and 18.7 degrees 2-theta.

209. The composition of claim 207 wherein the polymorph comprises four additional peaks at about 20.9, 21.4, 26.8, and 30.0 degrees 2-theta.

210. The composition of claim 209 wherein the polymorph comprises four additional peaks at about 11.4, 15.6, 25.0, and 26.1 degrees 2-theta.

211. The composition of claim 210 wherein the polymorph comprises three additional peaks at about 17.0, 21.8, and 22.0 degrees 2-theta.

212. The composition of claim 201 or claim 202, wherein the composition comprises a polymorph of N⁴-(cyclopropylmethyl)-6-[(3R)-3-(methylamino)pyrrolidin-1-yl]pyrimidine-2,4-diamine tartrate dihydrate distinguished by PXRD peaks at about 17.0, 21.8, and 26.1 degrees 2-theta.

213. The composition of claim 201, wherein the composition comprises a polymorph of N4-(cyclopropylmethyl)-6-[(3R)-3-(methylamino)pyrrolidin-1 yl]pyrimidine-2,4-diamine tartrate dihydrate distinguished by PXRD peaks at about 17.0, 21.8, and 26.1 degrees 2-theta.

214. The composition of claim 202, wherein the composition comprises a polymorph of N4-(cyclopropylmethyl)-6-r[3R)-3-(methylamino)pyrrolidin-1-yl]pyrimidine-2,4-diamine tartrate dihydrate distinguished by PXRD peaks at about 17.0, 21.8, and 26.1 degrees 2-theta.

215. A pharmaceutical composition comprising the composition of claim 201 or 202 or 206 and one or more pharmaceutically acceptable carrier(s) or diluent(s).

216. A dosage form comprising an effective amount of the composition of claim 201 or claim 202 or the pharmaceutical composition of claim 215 wherein the dosage form is selected from the group consisting of powder-in-capsule forms, capsules, tablets, liquids, powders, lozenges, chews, multi- and nano-particulates, gels, solid solutions, liposomes, nanoparticles, films, ovules, sprays, injectables, and liquid formulations.

217. The dosage form of claim 216, wherein the dosage form is a powder-in-capsule form.

218. The dosage form of claim 216, wherein the dosage form is a tablet.

219. The composition of claim 201, the pharmaceutical composition of claim 215, or the dosage form of claim 216 for use in the treatment of an H₄ mediated disease or condition.

220. The composition of claim 201, the pharmaceutical composition of claim 215, or the dosage form of claim 216 for use in the treatment of an H₄ mediated disease or condition, wherein the H₄ mediated disease or condition is selected from the group consisting of inflammatory skin diseases, pruritic diseases, respiratory diseases, cardiac diseases, inflammatory diseases of the gastrointestinal tract, cancer, joint diseases, kidney diseases, pain disorders, overactive bladder conditions, vestibular disorders, macular degenerative disorders, inflammatory eye diseases, and other diseases involving immune and inflammatory disorders.

221. The composition of claim 220, the pharmaceutical composition of claim 215, or the dosage form of claim 216, wherein the inflammatory skin disease or condition is psoriasis, atopic dermatitis, or other pruritic conditions.
